(19) United States
(12) Patent Application Publication
(10) Pub. No.: US 2026/0347798 A1
(43) Pub. Date: Nov. 5, 2026

(54) ENGINEERED KETOREDUCTASE POLYPEPTIDES

(71) Applicant: Novartis AG, Basel (CH)

(72) Inventors: Ning Ye, Changshu, Jiangsu (CN); Leo Albert Hardegger, Basel (CH); Lukas Simon Hueber, Basel (CH); Laura Leemans Martin, Basel (CH); Gregory Mann, Basel (CH); Theo Peschke, Basel (CH); Frédéric Valentin Stanger, Basel (CH); Oscar Alvizo, Redwood City, CA (US); Nandhitha Subramanian, Redwood City, CA (US)

(21) Appl. No.: 19/147,449

(22) PCT Filed: Jan. 10, 2024
(86) PCT No.: PCT/IB2024/050231

§ 371 Date: Jul. 11, 2025

(30) Foreign Application Priority Data
Jan. 12, 2023 (WO) PCT/CN2023/071908

(51) Int. Cl. C12N 9/04; C12P 17/10

(52) U.S. Cl. CPC C12N 9/0006; C12P 17/10; C12Y 101/01184

(57) ABSTRACT

The present disclosure provides engineered ketoreductase enzymes having improved enzymatic activity including the ability of selectively reducing tert-butyl rel-(3aR,6aS)-5-oxohexahydrocyclopenta[c]pyrrole-2(1H)-carboxylate to tert-butyl rel-(3aR,5s,6aS)-5-hydroxyhexahydrocyclopenta[c]pyrrole-2(1H)-carboxylate. Also provided are polynucleotides encoding the engineered ketoreductase enzymes, host cells capable of expressing the engineered ketoreductase enzymes, and methods of using the engineered ketoreductase enzymes.

### FIELD OF THE INVENTION

The present invention is related to the field of enzymology, and particularly to the field of ketoreductase enzymology. More specifically, the present invention is directed to ketoreductase polypeptides having improved enzymatic activity, and to the polynucleotide sequences that encode for the improved ketoreductase polypeptides.

### SEQUENCE LISTING

The instant application contains a Sequence Listing, which has been submitted electronically in XML format and is hereby incorporated by reference in its entirety. Said XML copy, created on Aug. 3, 2022, is named PAT059412-WO-PCT_SL.xml and is 866,783 bytes in size.

### BACKGROUND OF THE INVENTION

Enzymes belonging to the ketoreductase (KRED) or carbonyl reductase class (EC 1.1.1.184) are useful for the synthesis of optically active alcohols from the corresponding ketone substrate.

KREDs typically convert ketone and aldehyde substrates to the corresponding alcohol product, but may also catalyze the reverse reaction, oxidation of an alcohol substrate to the corresponding ketone/aldehyde product. The reduction of ketones and aldehydes and the oxidation of alcohols by enzymes such as KRED requires a cofactor, most commonly reduced nicotinamide adenine dinucleotide (NADH) or reduced nicotinamide adenine dinucleotide phosphate (NADPH), and nicotinamide adenine dinucleotide (NAD) or nicotinamide adenine dinucleotide phosphate (NADP) for the oxidation reaction. NADH and NADPH serve as electron donors, while NAD and NADP serve as electron acceptors. It is frequently observed that ketoreductases and alcohol dehydrogenases accept either the phosphorylated or the non-phosphorylated cofactor (in its oxidized and reduced state), but not both.

KRED enzymes can be found in a wide range of bacteria and yeasts (for reviews, see Kraus and Waldman, 1995, Enzyme catalysis in organic synthesis, Vols. 1&2 VCH Weinheim; Faber, K., 2000, Biotransformations in organic chemistry, 4th Ed. Springer, Berlin Heidelberg New York; and Hummel and Kula, 1989, Eur. J. Biochem. 184: 1-13). Several KRED gene and enzyme sequences have been reported (e.g., Candida magnoliae (Genbank Ace. No. JC7338; GI: 11360538); Candida parapsilosis (Genbank Ace. No. BAA24528.1; GI:2815409); Sporobolomyces salmonicolor (Genbank Ace. No. AF160799; GL6539734)).

In order to circumvent many chemical synthetic procedures for the production of key compounds, ketoreductases are being increasingly employed for the enzymatic conversion of different ketone and aldehyde substrates to chiral alcohol products. These applications can employ whole cells expressing the ketoreductase for biocatalytic ketone and aldehyde reductions, or by use of purified enzymes in those instances where presence of multiple ketoreductases in whole cells would adversely affect the stereopurity and yield of the desired product. For in vitro applications, a cofactor (NADH or NADPH) regenerating enzyme such as glucose dehydrogenase (GDH), formate dehydrogenase or second ketoreductase, etc. is used in conjunction with the ketoreductase. Examples using ketoreductases to generate useful chemical compounds include asymmetric reduction of 4-chloroacetoacetate esters (Zhou, 1983, J. Am. Chem. Soc. 105:5925-5926; Santaniello, J. Chem. Res. (S) 1984: 132-133; U.S. Pat. Nos. 5,559,030, 5,700,670, and 5,891,685), reduction of dioxocarboxylic acids (e.g., U.S. Pat. No. 6,399,339); reduction of tert-butyl (S) chloro-5-hydroxy-3-oxohexanoate (e.g., U.S. Pat. No. 6,645,746 and International Pub. No. WO 01/40450), reduction pyrrolotriazine-based compounds (e.g., U.S. Pub. No. 2006/0286646); reduction of substituted acetophenones (e.g., U.S. Pat. No. 6,800,477); and reduction of ketothiolanes (International Pub. No. WO 2005/054491).

Standard hydride reducing agents, e.g., lithium aluminum hydrides, sodium borohydrides, are reported to reduce bicyclic ketones to the corresponding secondary alcohols. However, for compounds of structure IA, these reagents predominantly (e.g., 9:1 ratio) produce the undesired diastereoisomer, i.e., the hydride gets delivered to the convex face to produce the hydroxyl which is trans relative to the hydrogens at the ring junction.

Some engineered ketoreductases also have activity to dehydrogenate a secondary alcohol reductant, e.g. iPrOH. In such cases, using secondary alcohol as reductant, the engineered ketoreductase and the secondary alcohol dehydrogenase are the same enzyme.

It is therefore desirable to identify other ketoreductase enzymes that can be used to carryout conversion of various keto substrates to the corresponding chiral alcohol products especially in the field of which are less prone to selective reductions on the most hindered face due to their U-shape nature. In addition, it is desirable to identify ketoreductases that can utilize isopropanol as stoichiometric sources of reductant for cofactor regeneration instead of using the well-known glucose dehydrogenase glucose system.

### SUMMARY OF THE INVENTION

It has been found that KRED enzymes, e.g., the KRED enzymes disclosed herein are diastereoselective in the reduction of bicyclic ketone compounds. The reduction occurs with delivery of the hydride from the concave face of the bicyclic ketone to install the hydroxyl group cis to the hydrogens at the ring junction. The engineered KRED polypeptides of the disclosure are surprisingly diastereoselective in the reduction of bicyclic ketone substrates to a (cis) alcohol product with a selectivity of at least 96%, with exemplary polypeptides of the disclosure having at least 99% diastereoselectivity for the (cis) alcohol product. Remarkably, the engineered KRED polypeptides of the disclosure do not require the use of DMSO in the reduction reaction, and isopropanol (iPrOH) may instead be used up to 40 wt %. By eliminating DMSO and increasing iPrOH in the reaction, the engineered KRED polypeptides of the disclosure are capable of a higher conversion rate, which ranges from 85% up to full conversion. Since iPrOH may be used at a higher concentration, the engineered KRED polypeptides of the disclosure unexpectedly do not require a GDH/glucose cofactor recycling system, which is required for other KRED enzymes in the reduction of bicyclic ketone compounds.

Provided herein are processes for the preparation of compounds useful in the treatment of diseases and disorders associated with depression, such as major depressive disorder and treatment resistant or refractory depression. In some embodiments, the prepared compounds include inhibitors of NR2B-NMDA receptors useful in the treatment of such diseases and disorders. In some embodiments, the prepared compounds include compounds that are exemplified, for example, in PCT Patent Application Publication WO/2016/049165, the content of which is incorporated herein in its entirety. The processes described herein, for example, improve product purity, diastereomeric ratio (dr), stereomeric excess, and/or yield of the final products as well as key intermediates in the synthesis thereof. The processes described herein will be more fully understood with reference to the several reaction schemes below. In some embodiments, the processes unexpectedly provide improved product purity, improved diastereomeric ratio, improved stereomeric excess, and/or improved yield. Improved product purity includes, for example, improved diastereomeric purity of the reaction product.

In one aspect, the present invention features engineered ketoreductase polypeptides. In some embodiments, an engineered ketoreductase polypeptide of the present invention is a polypeptide comprising an amino acid sequence having at least about 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% sequence identity to an amino acid sequence selected from the group consisting of SEQ ID NOs: 392, 394, 396, 406, 408, 420, 422, 424, 426, 428, 430, 436, 438, 462, 464, 468, 470, 474, 476, 480, 482, and 490. In some embodiments, an engineered ketoreductase polypeptide of the present invention is a polypeptide comprising an amino acid sequence having (1) at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NOs: 54, 152, 256, or 492; and (2) comprising one or more amino acid differences relative to said amino acid sequence selected from: i) X17S, X17T, or X17G; ii) X18S or X18R; iii) X56S, X56T, or X56D; iv) X94I; v) X106I, X106V, X106L, or X106W; vi) X173A, X173W, X173M, or X173K; vii) X194A, X194V, X194W, or X194T; viii) X196G; and ix) X198V, X198I, X198Y, or X198P, optionally comprising one or more additional amino acid residue differences relative to said amino acid sequence. In some embodiments, an engineered ketoreductase polypeptide of the present invention is a polypeptide comprising an amino acid sequence selected from Table 4 (SEQ ID NOs: 4-52), Table 5 (SEQ ID NOs: 56-186), Table 6 (SEQ ID NOs: 188-390) or Table 7 (SEQ ID NOs: 392-490). In some embodiments, the polypeptide is capable of selectively reducing tert-butyl rel-(3aR,6aS)-5-oxohexahydrocyclopenta[c]pyrrole-2(1H)-carboxylate (Substrate) to tert-butyl rel-(3aR,5s,6aS)-5-hydroxyhexahydrocyclopenta[c]pyrrole-2(1H)-carboxylate (Product).

In another aspect, the present invention features an engineered ketoreductase polypeptide capable of selectively reducing tert-butyl rel-(3aR,6aS)-5-oxohexahydrocyclopenta[c]pyrrole-2(1H)-carboxylate (Substrate) to tert-butyl rel-(3aR,5s,6aS)-5-hydroxyhexahydrocyclopenta[c]pyrrole-2(1H)-carboxylate (Product). In some embodiments, the engineered ketoreductase polypeptide of the present invention comprises an amino acid sequence having (i) at least about 80%, 85%, 90%, 95%, 96%, 97%, 98%, 99%, or 100% sequence identity to SEQ ID NO: 54, 152, 256, or 492 and (ii) a substitution, deletion, addition or insertion of one or more amino acid residues selected from Table 2, Table 4, Table 5, Table 6, or Table 7 relative to said amino acid sequence, optionally comprising one or more additional amino acid residue differences relative to said amino acid sequence.

In some embodiments, the engineered polypeptide amino acid sequence comprises one or more amino acid differences at positions X17, X18, X40, X56, X94, X96, X106, X145, X173, X190, X194, X196, X198, X202, and/or X206 relative to said amino acid sequence, optionally comprising one or more additional amino acid residue differences relative to said amino acid sequence. In some embodiments, the engineered polypeptide amino acid sequence comprises one or more of the following amino acid residues: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198I, X198Y, X198D, X198P, X202A, X202G, X206Q, X206N and/or X206W relative to said amino acid sequences, optionally comprising one or more additional amino acid residue differences relative to said amino acid sequence. In some embodiments, the engineered polypeptide amino acid sequence comprises one or more amino acid residues selected from X94I, X96Y, X190A, X196G, X202A, X202G and/or X206W relative to said amino acid sequences, optionally comprising one or more additional amino acid residue differences relative to said amino acid sequence. In some embodiments, the engineered polypeptide amino acid sequence comprises one or more amino acid residues selected from X18G, X40H, X56V, X94A, X106E, X145E, X145, X173D, X194P, X198D, X202A, and X206Q relative to said amino acid sequences, optionally comprising one or more additional amino acid residue differences relative to said amino acid sequence.

In some embodiments, the engineered polypeptide amino acid sequence comprises (i) a residue at position X190 that is not tyrosine; (ii) a residue at position X190 that is a non-aromatic residue; (iii) a residue at position X196 that is an aliphatic residue or a small amino acid residue; (iv) a residue at position X202 that is a small amino acid residue; and/or (v) a residue at position X206 that is not methionine, relative to said amino acid sequences, optionally comprising one or more additional amino acid residue differences relative to said amino acid sequence.

In some embodiments, the engineered polypeptide amino acid sequence comprises an amino acid sequence selected from the group consisting of: SEQ ID NOs: 392, 394, 396, 406, 408, 420, 422, 424, 426, 428, 430, 436, 438, 462, 464, 468, 470, 474, 476, 480, 482, and 490.

In some embodiments, the engineered polypeptide is solvent stable. In some embodiments, the engineered polypeptide reduces Substrate to Product with a conversion rate of at least about 85%, at least about 90%, at least about 95%, at least about 96%, at least about 97%, at least about 98%, at least about 99.0%, or 100%. In some embodiments, the engineered polypeptide reduces Substrate to Product with a level of selectivity (% diastereomeric excess) of at least about 96.0%, at least about 96.5%, at least about 97.0%, at least about 97.5%, at least about 98.0%, at least about 98.5%, at least about 99.0%, at least about 99.5%, or 100%. In some embodiments, the capability of reducing Substrate to Product is relative to a reference (e.g., parental) polypeptide. In some embodiments, the engineered polypeptide has a reversed or increased diastereoselectivity relative to the reference (e.g., parental) polypeptide for reducing Substrate to Product. In some embodiments, the engineered polypeptide has a level of increased activity (e.g., conversion rate or desired product) relative to the reference (e.g., parental) polypeptide with a fold improvement over positive control (FIOP) greater than about 1.75, greater than about 2.00, greater than about 2.25, greater than about 2.50, greater than about 2.75, greater than about 3.00, greater than about 3.25, greater than about 3.50, greater than about 3.75, greater than about 4.00, greater than about 4.25, greater than about 4.50, greater than about 4.75, greater than about 5.00, greater than about 5.25, greater than about 5.50, greater than about 5.75, greater than about 6.00, greater than about 6.25, or greater than about 6.50. In some embodiments, the engineered polypeptide is capable of reducing Substrate to Product with a FIOP conversion rate greater than about 2.25, preferably greater than about 3.00, and a diastereoselectivity greater than about 95%, preferably greater than about 97%, or more preferably greater than about 99%, as compared to the reference polypeptide.

In some embodiments, the reference (e.g., parental) polypeptide is a wild-type Lactobacillus kefir, Lactobacillus brevis, or Lactobacillus minor ketoreductase polypeptide. In some embodiments, the reference (e.g., parental) polypeptide is a wild-type Lactobacillus kefir ketoreductase polypeptide. In some embodiments, the reference (e.g., parental) polypeptide is a wild-type Lactobacillus kefir ketoreductase polypeptide comprising an amino acid sequence of SEQ ID NO: 492. In some embodiments, the reference (e.g., parental) polypeptide is an engineered ketoreductase polypeptide. In some embodiments, the reference (e.g., parental) polypeptide is an engineered ketoreductase polypeptide selected from SEQ ID NO: 54, SEQ ID NO: 152, or SEQ ID NO: 256.

In some embodiments, the engineered polypeptide i) requires less cofactor; ii) does not require glucose dehydrogenase (GDH)/glucose cofactor recycling; and/or iii) does not require dimethylsulfoxide (DMSO), as compared to the reference (e.g., parental) polypeptide in a reduction reaction for reducing Substrate to Product.

In one aspect, the present invention features engineered ketoreductase polypeptides capable of selectively reducing tert-butyl rel-(3aR,6aS)-5-oxohexahydrocyclopenta[c]pyrrole-2(1H)-carboxylate (Substrate) to tert-butyl rel-(3aR,5s,6aS)-5-hydroxyhexahydrocyclopenta[c]pyrrole-2(1H)-carboxylate (Product) under suitable reaction conditions at a greater stereoselectivity (% diastereomeric excess) and/or activity (% conversion) than that of SEQ ID NO: 54, SEQ ID NO: 152, and/or SEQ ID NO: 256.

In some embodiments, suitable reaction conditions include one or more of the following: i) up to about 150 g/L, e.g., 50 g/L or 100 g/L, of Substrate; ii) enzyme loading less than about 5 wt %, e.g., less than about 3 wt %, e.g., less than about 1 wt %; iii) NADP+ cofactor loading less than about 2 wt %, e.g., less than about 1 wt %, e.g., less than about 0.2 wt %, e.g., 0.1%, 0.05%, or 0.03%; iv) up to 40% (v/v) of isopropanol; v) a temperature of about 15 to 75 °C, e.g., 20 to 55 °C, e.g., 20 to 45 °C, e.g., 35 °C or 40 °C; vi) a pH of 5.0 to 10.0, e.g., pH of 7.5-8.3; and vii) a reaction time of up to 30 hours, preferably 24 hours. In some embodiments, suitable reaction conditions do not require dimethylsulfoxide (DMSO). In some embodiments, suitable reaction conditions do not require GDH/Glucose cofactor recycling.

In some embodiments, the engineered polypeptide comprises an amino acid sequence that differs from the amino acid sequence of SEQ ID NO: 54, SEQ ID NO: 152, or SEQ ID NO: 256 by one or more amino acid residues selected from: Table 2, Table 4, Table 5, Table 6, or Table 7. In some embodiments, the engineered polypeptide comprises an amino acid sequence that differs from the amino acid sequence of SEQ ID NO: 54, SEQ ID NO: 152, or SEQ ID NO: 256 in one or more amino acid residues selected from: 17, 18, 40, 56, 94, 96, 106, 145, 173, 190, 194, 196, 198, 202, and/or 206. In some embodiments, the engineered polypeptide amino acid sequence comprises (i) a residue at position X190 that is not tyrosine; (ii) a residue at position X190 that is a non-aromatic residue; (iii) a residue at position X196 that is an aliphatic residue or a small amino acid residue; (iv) a residue at position X202 that is a small amino acid residue; and/or (v) a residue at position X206 that is not methionine, relative to the amino acid sequence of SEQ ID NO: 54, SEQ ID NO: 152, or SEQ ID NO: 256. In some embodiments, the engineered polypeptide amino acid sequence comprises one or more amino acid residues selected from: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198I, X198Y, X198D, X198P, X202A, X202G, X206Q, X206N and/or X206W, relative to the amino acid sequence of SEQ ID NO: 54, SEQ ID NO: 152, or SEQ ID NO: 256. In some embodiments, the engineered polypeptide amino acid sequence comprises one or more amino acid residues selected from: i) X17S, X17T, or X17G; ii) X18S or X18R; iii) X56S, X56T, or X56D; iv) X94I; v) X106I, X106V, X106L, or X106W; vi) X173A, X173W, X173M, or X173K; vii) X194A, X194V, X194W, or X194T; viii) X196G; and ix) X198V, X198I, X198Y, or X198P; relative to the amino acid sequence of SEQ ID NO: 54, SEQ ID NO: 152, or SEQ ID NO: 256. In some embodiments, the engineered polypeptide amino acid sequence comprises one or more amino acid residues selected from: X94I, X96Y, X190A, X196G, X202A, X202G and/or X206W, relative to the amino acid sequence of SEQ ID NO: 54, SEQ ID NO: 152, or SEQ ID NO: 256. In some embodiments, the engineered polypeptide amino acid sequence comprises one or more amino acid residues selected from: X18G, X40H, X56V, X94A, X106E, X145E, X145, X173D, X194P, X198D, X202A, and X206Q, relative to the amino acid sequence of SEQ ID NO: 54, SEQ ID NO: 152, or SEQ ID NO: 256. In some embodiments, the engineered polypeptide comprises an amino acid sequence selected from: a) an amino acid sequence selected from Table 4 (SEQ ID NOs: 4-52), Table 5 (SEQ ID NOs: 56-186), Table 6 (SEQ ID NOs: 188-390), or Table 7 (SEQ ID NOs: 392-490). In some embodiments, the engineered polypeptide comprises an amino acid sequence selected from Table 7. In some embodiments, the engineered polypeptide comprises an amino acid sequence having at least about 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% sequence identity to an amino acid sequence selected from the group consisting of SEQ ID NOs: 392, 394, 396, 406, 408, 420, 422, 424, 426, 428, 430, 436, 438, 462, 464, 468, 470, 474, 476, 480, 482, and 490. In some embodiments, the amino acid sequence of the engineered polypeptide includes one or more additional amino acid residue differences relative to SEQ ID NO: 54, SEQ ID NO: 152, or SEQ ID NO: 256.

In another aspect, the present invention features methods for the diastereoselective reduction of a bicyclic ketone. In some embodiments, provided is a method for the diastereoselective reduction of a bicyclic ketone, the method comprising the step of contacting the bicyclic ketone with a KRED, under suitable reaction conditions, to obtain a bicyclic secondary alcohol product.

In some embodiments, the KRED is an engineered ketoreductase.

In some embodiments, the bicyclic ketone substrate has between 6 and 12 members in total.

In some embodiments, the bicyclic ketone substrate is achiral.

In some embodiments, the method includes the step of contacting a bicyclic ketone substrate with any of the engineered polypeptides of the present invention under suitable reaction conditions to obtain a bicyclic secondary alcohol product. In some embodiments, the resulting bicyclic secondary alcohol product has the structure shown in formula (IB):

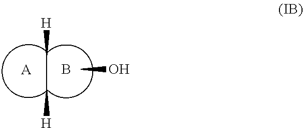

wherein:
the A ring and the B ring together represent a fused cycloalkyl ring, e.g., C₆-C₁₂ cycloalkyl, or fused heterocyclyl ring, e.g., 6-12 membered heterocyclyl,
wherein the fused cycloalkyl or heterocyclyl is optionally substituted with at least one occurrence of R¹, e.g., one to four R¹,
each R¹ is independently selected from an amine protecting group, e.g., tert-butyloxycarbonyl (Boc), N-carboxybezyl (Cbz), C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, 5-20 membered heteroaryl, hydroxyl, halogen, e.g., F, C₁-C₂₀haloalkyl, e.g., —CF₃, —OR^{c}, —NR^{c}R^{c}, —(CH₂)ₙCOOR^{c}, —(CH₂)ₙ—C(═O)R^{c}, and —(CH₂)ₙ—C(═O)NR^{c}R^{c};
wherein the alkyl, alkenyl, and alkynyl are each optionally substituted by one or more R^{a}, e.g., one to six R^{a}, and wherein the cycloalkyl, aryl, arylalkyl, heterocyclyl, and heteroaryl are each optionally substituted by one or more R^{b}, e.g., one to six R^{b};
each R^{a} is at each occurrence independently selected from C₃-C₁₀cycloalkyl, C₆-C₁₄aryl, C₇-C₂₀arylalkyl, 3-14 membered heterocyclyl, and 5-20 membered heteroaryl, halogen, e.g., F, haloalkyl, e.g., C₁-C₂₀haloalkyl, e.g., —CF₃, —OR^{c}, —NR^{c}R^{c}, —(CH₂)ₙCOOR^{c}, —(CH₂)ₙ—C(═O)R^{c}, and —(CH₂)ₙ—C(═O)NR^{c}R^{c};
each R^{b} is at each occurrence independently selected from halogen, C₁-C₂₀haloalkyl, e.g., —CF₃, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c}, C₁-C₂₀alkyl, C₂-C₂₀alkenyl, and C₂-C₂₀alkynyl;
each R^{c} is at each occurrence independently selected from H, C₁-C₂₀alkyl, C₂-C₂₀alkenyl, and C₂-C₂₀alkynyl, each optionally substituted by one or more R^{b}, e.g., one to six R^{b}; and
n is 0, 1, 2, 3, 4, 5 or 6, e.g., 0, 1, 2 or 3.

In some embodiments, the bicyclic ketone substrate has the structure shown in formula (IA):

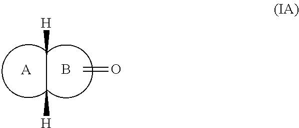

wherein A and B are as defined in relation to formula (IB) above.

In some embodiments, the A ring and the B ring together represent a fused 6-12 membered heterocyclyl comprising at least one nitrogen heteroatom. In some embodiments, the nitrogen heteroatom of the bicyclic ketone substrate is bonded to an amine protecting group, e.g., tert-butyloxycarbonyl (Boc), N-carboxybenzyl (Cbz).

In some embodiments, the bicyclic ketone substrate is of formula (IA)-I:

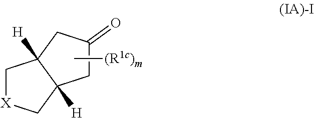

wherein:
X is selected from N—R^{1a}, CH₂ and CH—R^{1b};
R^{1a} is selected from an amine protecting group, e.g., tert-butyloxycarbonyl (Boc), N-carboxybenzyl (Cbz), C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, and 5-20 membered heteroaryl;
R^{1b} is selected from C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, and 5-20 membered heteroaryl;
wherein the alkyl, alkenyl, and alkynyl of R^{1a} or R^{1b} are each optionally substituted by one to six R^{a}; and
wherein the cycloalkyl, aryl, arylalkyl, heterocyclyl, and heteroaryl of R^{1a} or R^{1b} are each optionally substituted by one to six R^{b};
each R^{1c} is at each occurrence independently selected from C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, 5-20 membered heteroaryl, hydroxyl, halogen, e.g., F, C₁-C₂₀haloalkyl, e.g., —CF₃, —OR^{c}, —NR^{c}R^{c}, —(CH₂)ₙCOOR^{c}, —(CH₂)ₙ—C(═O)R^{c}, and —(CH₂)ₙ—C(═O)NR^{c}R^{c};
each R^{a} is at each occurrence independently selected from C₃-C₁₀cycloalkyl, C₆-C₁₄aryl, C₇-C₂₀arylalkyl, 3-14 membered heterocyclyl, and 5-20 membered heteroaryl, halogen, e.g., F, haloalkyl, e.g., C₁-C₂₀haloalkyl, e.g., —CF₃, —OR^{c}, —NR^{c}R^{c}, —(CH₂)ₙCOOR^{c}, —(CH₂)ₙ—C(═O)R^{c}, and —(CH₂)ₙ—C(═O)NR^{c}R^{c};
each R^{b} is at each occurrence independently selected from halogen, C₁-C₂₀haloalkyl, e.g., —CF₃, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c}, C₁-C₂₀alkyl, C₂-C₂₀alkenyl, and C₂-C₂₀alkynyl;
each R^{c} is at each occurrence independently selected from H, C₁-C₂₀alkyl, C₂-C₂₀alkenyl, and C₂-C₂₀alkynyl, each optionally substituted by one to six R^{b};
n is 0, 1, 2 or 3; and
m is 0, 1 or 2.

In some embodiments, X is N—R^{1a}; R^{1a} is selected from C₁-C₁₀alkyl, C₃-C₁₀cycloalkyl, C₆-C₁₄aryl, C₇-C₂₀arylalkyl, and an amine protecting group, e.g., tert-butyloxycarbonyl (Boc), N-carboxybenzyl (Cbz); and m is 0. In some embodiments, X is N-Ria, and Ria is an amine protecting group, e.g., tert-butyloxycarbonyl (Boc), N-carboxybenzyl (Cbz).

In another aspect, the present invention features methods for stereoselectively reducing (IA)-I to (IB)-I:

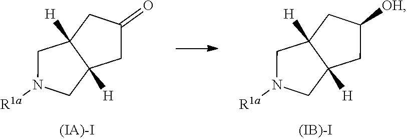

wherein R^{1a} is an amine protecting group. In a particular embodiment, R^{1a} is selected from tert-butyloxycarbonyl (Boc) and N-carboxybenzyl (Cbz). In some embodiments, the method includes the step of contacting the substrate (IA)-I, with a KRED, under reaction conditions suitable for reducing or converting (IA)-I to (IB)-I. In some embodiments, the KRED is an engineered ketoreductase. In some embodiments, the KRED is an engineered polypeptide as described herein.

In another aspect, the present invention features methods for stereoselectively reducing tert-butyl rel-(3aR,6aS)-5-oxohexahydrocyclopenta[c]pyrrole-2(1H)-carboxylate (substrate) to tert-butyl rel-(3aR,5s,6aS)-5-hydroxyhexahydrocyclopenta[c]pyrrole-2(1H)-carboxylate (product):

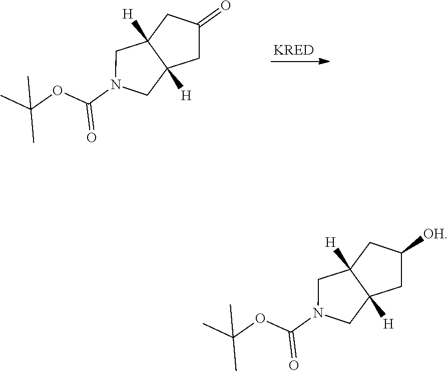 [SMILES: CC(C)(C)OC(=O)N1C[C@H]2CC(=O)C[C@H]2C1 ; CC(C)(C)OC(=O)N1C[C@H]2C[C@H](O)C[C@H]2C1]

In some embodiments, the method includes the step of contacting the substrate (IA)-I, e.g., tert-butyl rel-(3aR,6aS)-5-oxohexahydrocyclopenta[c]pyrrole-2(1H)-carboxylate with a KRED under reaction conditions suitable for reducing or converting (IA)-I, e.g., tert-butyl rel-(3aR,6aS)-5-oxohexahydrocyclopenta[c]pyrrole-2(1H)-carboxylate (substrate) to (IB)-I, e.g., tert-butyl rel-(3aR,5s,6aS)-5-hydroxyhexahydrocyclopenta[c]pyrrole-2(1H)-carboxylate (product). In some embodiments, the KRED is an engineered ketoreductase. In some embodiments, the KRED is an engineered polypeptide as described herein.

In some embodiments, the reaction is carried out in a solvent. In some embodiments, the solvent is selected from a polar solvent, non-polar solvent and ionic liquid. In some embodiments, the solvent is selected from water, methanol, ethanol, n-propanol, isopropanol, isopropyl acetate, dimethyl sulfoxide, dimethylformamide, ethyl acetate, butyl acetate, 1-octanol, hexane, heptane, octane, methyl tert-butyl ether, toluene, 1-ethyl-4-methylimidazolium tetrafluoroborate, 1-butyl-3-methylimidazolium tetrafluoroborate, 1-butyl-3-methylimidazolium hexafluorophosphate, glycerol, ethylene glycol, propylene glycol and polyethylene glycol. In some embodiments, the solvent is isopropanol. In some embodiments, the reaction is carried out in up to 40 wt % of isopropanol. In some embodiments, the reaction is carried out in the presence of a co-solvent. In some embodiments, the reaction is carried out in an aqueous co-solvent system. In some embodiments, the co-solvent is selected from dimethylsulfoxide (DMSO), and an alcohol, e.g., methanol, ethanol, n-propanol, isopropanol. In some embodiments, the reaction is not carried out in the presence of dimethylsulfoxide (DMSO).

In some embodiments, the reaction is carried out at a temperature of 15 to 75 °C. In some embodiments, the reaction is carried out at a temperature of 20 to 55 °C. In some embodiments, the reaction is carried out at a temperature of 20 to 45 °C, e.g., 35 °C or 40 °C. In some embodiments, the reaction is carried out at a pH of 5.0 to 10.0, e.g., 7.5-8.3. In some embodiments, the reaction is carried out at a pH of 7.5.

In some embodiments, the bicyclic ketone substrate is present at a loading concentration up to about 150 g/L. In some embodiments, the concentration of the bicyclic ketone substrate is at least about 5 g/L, at least about 10 g/L, at least about 20 g/L, at least about 50 g/L, at least about 100 g/L, or about 150 g/L.

In some embodiments, the polypeptide is present at a concentration less than about 10 g/L. In some embodiments, the concentration of the polypeptide is less than about 5 g/L, e.g., less than about 5 g/L, e.g., less than about 3 g/L, e.g., less than about 1 g/L. In some embodiments, the solvent is present at a concentration of 20% to 40% v/v.

In some embodiments, the method is carried out with whole cells that express the ketoreductase enzyme, or an extract or lysate of such cells. In some embodiments, the method does not require glucose dehydrogenase (GDH)/glucose cofactor recycling.

In some embodiments, the ketoreductase is isolated and/or purified and the reduction reaction is carried out in the presence of a cofactor for the ketoreductase. In some embodiments, the cofactor comprises nicotinamide adenine dinucleotide (NADH) or nicotinamide adenine dinucleotide phosphate (NADPH). In some embodiments, the cofactor is present at a concentration of less than about 2 wt %, e.g., less than about 1 wt %, e.g., less than about 0.2 wt %, e.g., 0.1%, 0.05%, or 0.03%.

In some embodiments, the method results in the product with a selectivity (% diastereomeric excess) greater than about 96%, 97%, 98%, 99%, 99.1%, 99.2%, 99.3%, 99.4%, 99.5%, 99.6%, 99.7%, 99.8% or 99.9% and/or a conversion % of at least about 85%, at least about 90%, at least about 95%, at least about 96%, at least about 97%, at least about 98%, at least about 99.0%, or 100%. In some embodiments, at least about 90% of the substrate is reduced to the product in less than about 20 hours. In some embodiments, at least about 85% of the substrate is reduced to product in less than about 20 hours and at least about 95% of the substrate is reduced to the product in less than about 30 hours.

In one aspect, the present invention features methods for synthesizing 6-((S)-2-((3aR,5R,6aS)-5-(2-fluorophenoxy)hexahydrocyclopenta[c]pyrrol-2(1H)-yl)-1-hydroxyethyl)pyridin-3-ol, in free form or in pharmaceutically acceptable salt form, of formula (IC)

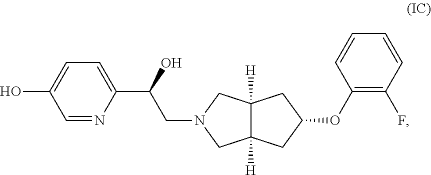 [SMILES: Oc1ccc([C@@H](O)CN2C[C@H]3C[C@H](Oc4ccccc4F)C[C@H]3C2)nc1]

In some embodiments, the method includes a process that includes the step of contacting a substrate of formula (IA)-I, such as tert-butyl rel-(3aR,6aS)-5-oxohexahydrocyclopenta[c]pyrrole-2(1H)-carboxylate (Substrate) with a KRED under reaction conditions suitable for reducing or converting (IA)-I, such as tert-butyl rel-(3aR,6aS)-5-oxohexahydrocyclopenta[c]pyrrole-2(1H)-carboxylate (substrate) to the compound of formula (IB)-I, such as tert-butyl rel-(3aR,5s,6aS)-5-hydroxyhexahydrocyclopenta[c]pyrrole-2(1H)-carboxylate (product). In some embodiments, the KRED is an engineered KRED. In some embodiments, the KRED is an engineered polypeptide as described herein.

In another aspect, the present invention features methods for reversing the diastereoselectivity of a ketoreductase (KRED) polypeptide from the formation of (trans) alcohol product (e.g., (5r)-2) towards the formation of (cis) alcohol product (e.g., (5s)-2) in a reduction reaction. In yet another aspect, the present invention features methods for increasing the diastereoselectivity of a ketoreductase (KRED) polypeptide towards the formation of (cis) alcohol product (e.g., (5s)-2) in a reduction reaction. In some embodiments, the methods include introducing one or more amino acid differences selected from Table 2, Table 4, Table 5, Table 6, or Table 7 relative to the amino acid sequence of the KRED polypeptide, wherein the one or more amino acid differences reverse or increase the diastereoselectivity of the KRED polypeptide towards the formation of (cis) alcohol product (e.g., (5s)-2) when used in a reduction reaction compared to the KRED polypeptide without the one or more amino acid differences. In some embodiments, the one or more amino acid differences are selected from the following positions: 17, 18, 40, 56, 94, 96, 106, 145, 173, 190, 194, 196, 198, 202, and/or 206, relative to the KRED polypeptide amino acid sequence.

In some embodiments, the one or more amino acid differences are selected from the following: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198I, X198Y, X198D, X198P, X202A, X202G, X206Q, X206N and/or X206W, relative to the KRED polypeptide amino acid sequence. In some embodiments, the one or more amino acid differences are selected from: i) X17S, X17T, or X17G; ii) X18S or X18R; iii) X56S, X56T, or X56D; iv) X94I; v) X106I, X106V, X106L, or X106W; vi) X173A, X173W, X173M, or X173K; vii) X194A, X194V, X194W, or X194T; viii) X196G; and ix) X198V, X198I, X198Y, or X198P; relative to the KRED polypeptide amino acid sequence. In some embodiments, the one or more amino acid differences are selected from: X94I, X96Y, X190A, X196G, X202A, X202G and/or X206W, relative to the KRED polypeptide amino acid sequence. In some embodiments, the one or more amino acid differences are selected from: X18G, X40H, X56V, X94A, X106E, X145E, X145, X173D, X194P, X198D, X202A, and X206Q, relative to the KRED polypeptide amino acid sequence. In some embodiments, the one or more amino acid differences are selected from: (i) a residue at position X190 that is not tyrosine; (ii) a residue at position X190 that is a non-aromatic residue; (iii) a residue at position X196 that is an aliphatic residue or a small amino acid residue; (iv) a residue at position X202 that is a small amino acid residue; and/or (v) a residue at position X206 that is not methionine, relative to the KRED polypeptide amino acid sequence.

In some embodiments, the KRED polypeptide is a wild-type Lactobacillus kefir, Lactobacillus brevis, or Lactobacillus minor ketoreductase polypeptide. In some embodiments, the KRED polypeptide is a wild-type Lactobacillus kefir ketoreductase polypeptide. In some embodiments, the KRED polypeptide is a wild-type Lactobacillus kefir ketoreductase polypeptide comprising an amino acid sequence of SEQ ID NO: 492. In some embodiments, the KRED polypeptide is an engineered ketoreductase polypeptide. In some embodiments, the KRED polypeptide is an engineered ketoreductase polypeptide selected from Table 4, Table 5, or Table 6. In some embodiments, the KRED polypeptide is an engineered ketoreductase polypeptide selected from SEQ ID NO: 54, SEQ ID NO: 152, and SEQ ID NO: 256.

In some embodiments, the method results in a conversion to (cis) alcohol product (e.g., (5s)-2) of at least about 85%, at least about 90%, at least about 95%, at least about 96%, at least about 97%, at least about 98%, at least about 99.0%, or 100%. In some embodiments, the method results in a level of selectivity (% diastereomeric excess) of (cis) alcohol product (e.g., (5s)-2) of at least about 96.0%, at least about 96.5%, at least about 97.0%, at least about 97.5%, at least about 98.0%, at least about 98.5%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or 100%.

In a further aspect, there is provided a compound, which is

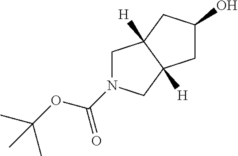 [SMILES: CC(C)(C)OC(=O)N1C[C@H]2C[C@H](O)C[C@H]2C1]

or a salt thereof.

In a further aspect, there is provided the use of a compound, which is

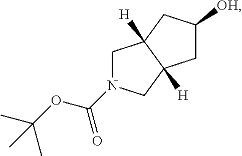 [SMILES: CC(C)(C)OC(=O)N1C[C@H]2C[C@H](O)C[C@H]2C1]

or a salt thereof, in the preparation of

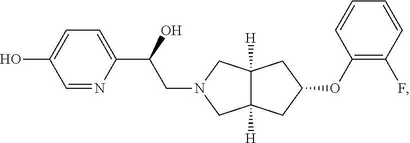 [SMILES: Oc1ccc([C@@H](O)CN2C[C@H]3C[C@H](Oc4ccccc4F)C[C@H]3C2)nc1]

in free form or in pharmaceutically acceptable salt form. In some embodiments, the compound (IC) is present in a % diastereomeric excess (de) of at least about 85%, at least about 96%, or at least about 99%.

In yet another aspect, the present invention features compositions comprising any of the engineered KRED polypeptides of the present invention. In some embodiments, the composition further includes the structural formula of Substrate and/or the compound of Product of the present disclosure.

In a further aspect, the present invention features immobilized polypeptides. In some embodiments, the polypeptide is selected from any of the engineered KRED polypeptides according to the present invention. In some embodiments, the polypeptide is immobilized to a solid support. In some embodiments, the polypeptide is immobilized to a solid support by a chemical bond (e.g., covalent or ionic bond), physical adsorption or affinity interactions. In some embodiments, the solid support is organic or inorganic, bearing different functional groups. Nonlimiting solid supports include resin, silica, zeolite, charcoal, celite (e.g., diatomaceous earth), synthetic polymers (e.g., polymethacrylate or the anion exchange resin Amberlite), biopolymers (e.g., cellulose, chitosan, agarose, lignin or lignocellulose), controlled pore glass, magnetic nanoparticles, metal-organic frameworks, or DNA, among many others. In some embodiments, the polypeptide is immobilized via entrapment in a hydrogel (e.g., alginate, chitosan, carrageenan) or a matrix (e.g., polyacrylamide). In some embodiments, the polypeptide is immobilized via carrier-free immobilization, e.g., cross-linking of the enzymes in the absence of support. This is achieved, for example, via precipitation of the enzymes as aggregates maintaining the tertiary structure, followed by cross-linking using glutaraldehyde.

In one aspect, the present invention features polynucleotides encoding any of the engineered KRED polypeptides according to the present invention. In some embodiments, the polynucleotide includes a nucleic acid sequence listed in Table 4 (SEQ ID NOs: 3-51), Table 5 (SEQ ID NOs: 55-185), Table 6 (SEQ ID NOs: 187-389), or Table 7 (SEQ ID NOs: 391-489).

In another aspect, the present invention features polynucleotides encoding an engineered KRED polypeptide, comprising a nucleic acid sequence that is at least about 98%, at least about 99%, or is 100% identical to a nucleic acid sequence selected from the group consisting of: SEQ ID NO: 391, 393, 395, 405, 407, 419, 421, 423, 425, 427, 429, 435, 437, 461, 463, 467, 469, 473, 475, 479, 481, and 489.

In yet another aspect, the present invention features expressions vector comprising any of the polynucleotides of the present invention operably linked to control sequences suitable for directing expression of the encoded polypeptide in a host cell. In some embodiments, the control sequence comprises a secretion signal.

In a further aspect, the present invention features host cells comprising any of the polynucleotides or expression vectors according to the present invention.

In one aspect, the present invention features methods of preparing an engineered ketoreductase polypeptide. In some embodiments, the method includes culturing any of the host cells of the present invention under conditions suitable for gene expression and thereafter purifying and collecting the engineered polypeptide thereof from the cell culture.

In another aspect, the present invention features kits. In some embodiments, the kits include any of the engineered KRED polypeptides according to the present invention. In some embodiments, the kits include any of the composition according to the present invention. In some embodiments, the kits include any of the polynucleotides according to the present invention. In some embodiments, the kits include any of the expression vectors according to the present invention. In some embodiments, the kits include any of the host cells according to the present invention. In some embodiments, the kits include any of the engineered KRED polypeptides according to the present invention, tert-butyl rel-(3aR,6aS)-5-oxohexahydrocyclopenta[c]pyrrole-2(1H)-carboxylate (Substrate), and a cofactor. In some embodiments, the cofactor is nicotinamide adenine dinucleotide (NADH) or nicotinamide adenine dinucleotide phosphate (NADPH). In some embodiments, the kit further includes instructions for use of any of the engineered polypeptides, compositions, polynucleotides, expression vectors, and/or host cells of the present invention.

Compositions defined by the invention were isolated or otherwise manufactured in connection with the examples provided below. The details of one or more embodiments of the invention are set forth herein. Other features and advantages of the invention will be apparent from the Detailed Description, the Examples, and the Claims.

### BRIEF DESCRIPTION OF THE DRAWINGS

FIG. 1 is a schematic depicting the role of ketoreductases (KRED) in the conversion of Compound 1 to compounds (5s)-2 or (5r)-2. This reduction uses a KRED of the invention and a cofactor such as NAD(P)H.

FIGS. 2A and 2B are graphs depicting the activity (% conversion) (FIG. 2A) and selectivity (% de) (FIG. 2B) of engineered KRED enzymes SEQ ID NO: 152, SEQ ID NO: 256, SEQ ID NO: 426 compared to a commercially available KRED enzyme (CM), parental KRED enzyme (SEQ ID NO: 54), and wild-type KRED enzyme (WT, SEQ ID NO: 492) under Condition 1 parameters at a substrate concentration of 50 g/L.

FIGS. 3A and 3B are graphs depicting the activity (% conversion) (FIG. 3A) and selectivity (% de) (FIG. 3B) of engineered KRED enzymes SEQ ID NO: 152, SEQ ID NO: 256, SEQ ID NO: 426 compared to a commercially available KRED enzyme (CM), parental KRED enzyme (SEQ ID NO: 54), and wild-type KRED enzyme (WT, SEQ ID NO: 492) under Condition 1 parameters at a substrate concentration of 90 g/L.

FIGS. 4A, 4B and 4C are graphs depicting the activity (% conversion) of engineered KRED enzymes SEQ ID NO: 152, SEQ ID NO: 256, SEQ ID NO: 426 compared to a commercially available KRED enzyme (CM), parental KRED enzyme (SEQ ID NO: 54), and wild-type KRED enzyme (WT, SEQ ID NO: 492) under Condition 2 parameters at a substrate concentration of 50 g/L (FIG. 4A), 100 g/L (FIG. 4B), and 150 g/L (FIG. 4C).

FIGS. 5A, 5B and 5C are graphs depicting the selectivity (% de) of engineered KRED enzymes SEQ ID NO: 152, SEQ ID NO: 256, and SEQ ID NO: 426 compared to a commercially available KRED enzyme (CM), parental KRED enzyme (SEQ ID NO: 54), and wild-type KRED enzyme (WT, SEQ ID NO: 492) under Condition 2 parameters at a substrate concentration of 50 g/L (FIG. 5A), 100 g/L (FIG. 5B), and 150 g/L (FIG. 5C).

### DETAILED DESCRIPTION OF THE INVENTION

Bicyclic secondary alcohols are useful intermediates in the synthesis of pharmacologically active agents. Tert-butyl rel-(3aR,5s,6aS)-5-hydroxyhexahydrocyclopenta[c]pyrrole-2(1H)-carboxylate, herein referred to as (substrate) and of the following structure

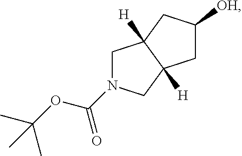 [SMILES: CC(C)(C)OC(=O)N1C[C@H]2C[C@H](O)C[C@H]2C1]

is especially useful in the synthesis of a compound of formula (IC).

Compound (IC), as disclosed herein, is 6-((1S)-2-((3aR,5R,6aS)-5-(2-fluorophenoxy)hexahydrocyclopenta[c]pyrrol-2(1H)-yl)-1-hydroxyethyl)pyridin-3-ol, of the following formula:

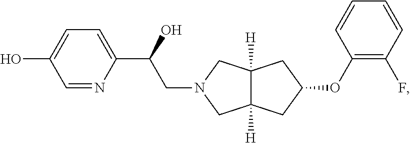 [SMILES: Oc1ccc([C@@H](O)CN2C[C@H]3C[C@H](Oc4ccccc4F)C[C@H]3C2)nc1]

known as onfasprodil and is a NR2B-NMDA receptor non-allosteric modulator (NAM) which can be prepared as described in WO2016/049165, incorporated herein by reference.

Evidence suggests that the NR2B negative allosteric modulators (NAMs) MK-0657 (also known as CERC-301) and CP-101,606 have low frequencies of dissociative adverse events (Garner et al. 2015; Pagnozzi et al. 1995; Preskorn et al. 2008). Although, the relative contribution of each individual subtype of NMDARs to the adverse effects of pan-NMDAR inhibition is poorly understood due to the lack of selective inhibitors for the various subtypes, taken together, this suggests that achieving safe, yet rapid-onset antidepressant efficacy is feasible with a compound selectively inhibiting NR2B receptor.

Compound (IC), or a pharmaceutically acceptable salt thereof, is a highly potent, selective and reversible low molecular weight NR2B-NMDA receptor NAM. Compound (I) is for the rapid reduction of depressive symptoms in patients with major depressive disorder (MDD), including treatment resistant depression and suicidality. This treatment is intended to allow patients to rapidly achieve a significant improvement of their depressive symptoms, and suicidality. Moreover, patients having MDD with suicidality, often require 4-5 days of hospitalization. Compound (I), having rapid-onset of efficacy, can reduce the number of days a patient is hospitalized and therefore provide a benefit over other antidepressants that take at least 4 weeks for a patient to respond to treatment.

Compound (IC), or a pharmaceutically acceptable salt thereof, is intended to treat suicidality, the symptoms of suicidality, including but not limited to, suicidal-ideation, suicidal-behavior and self-harm, alone or in conjunction with mental illness, including but not limited to, major depressive disorder. In particular, Compound (I), or a pharmaceutically acceptable salt thereof, is intended for the treatment of major depressive disorder in patients with suicidal ideation with intent.

Compound (IC) can be prepared by reducing bicyclic ketone (Int-1) with sodium borohydride in ethanol. The reduction process produces two diastereomers, 2 and 2A. Compound 2 is the undesired trans diastereoisomer and is formed as the major product. The undesired diastereomer 2 is then converted to the corresponding mesylate 18, which subsequently undergoes an SN² reaction as shown in Scheme 1 below.

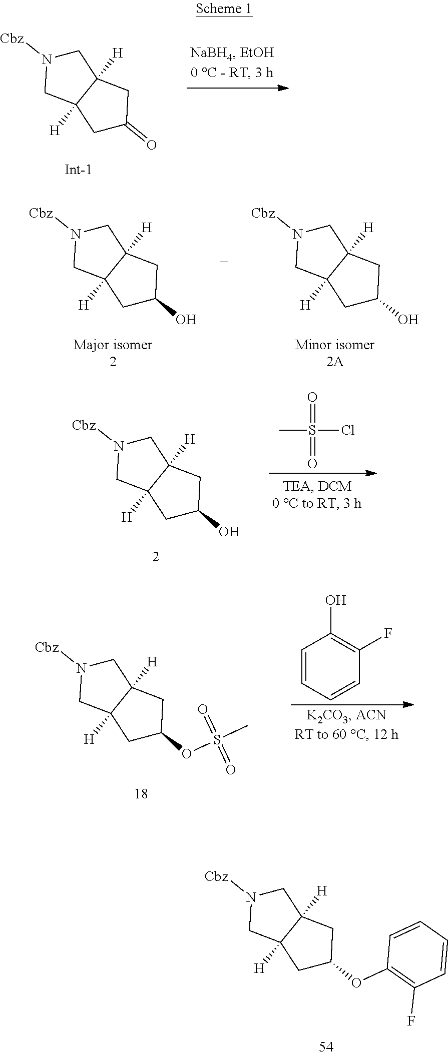

This reaction proceeds with inversion of stereochemistry at the C-5 position, to yield intermediate compound 54 of the proper stereochemistry. The process is disclosed in patent publication no. WO2016/049165. It has been reported that Compound 2A is produced in about 6.8% yield during the reduction process. Compound 54 is subsequently deprotected and alkylated with 2-bromo-1-(5-hydroxypyridin-2-yl)ethan-1-one, followed by a reduction reaction to give compound (IC).

There is therefore a need for improved methods for stereoselectively reducing bicyclic ketone substrates to their corresponding alcohol products. The KRED polypeptides disclosed herein and their use in the process of reducing bicyclic ketones to their corresponding secondary alcohol products provide a solution to this problem.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

“Acidic Amino Acid or Residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

By “agent” is meant any small molecule chemical compound, polynucleotide, polypeptide, or fragments thereof.

“Aliphatic Amino Acid or Residue” refers to a hydrophobic amino acid or residue having an aliphatic hydrocarbon side chain. Genetically encoded aliphatic amino acids include L-Ala (A), L-Val (V), L-Leu (L) and L-Ile (I).

“Aromatic Amino Acid or Residue” refers to a hydrophilic or hydrophobic amino acid or residue having a side chain that includes at least one aromatic or heteroaromatic ring. Genetically encoded aromatic amino acids include L-Phe (F), L-Tyr (Y) and L-Trp (W). Although owing to the pKa of its heteroaromatic nitrogen atom L-His (H) it is sometimes classified as a basic residue, or as an aromatic residue as its side chain includes a heteroaromatic ring, herein histidine is classified as a hydrophilic residue or as a “constrained residue.”

“Basic Amino Acid or Residue” refers to a hydrophilic amino acid or residue having a side chain exhibiting a pK value of greater than about 6 when the amino acid is included in a peptide or polypeptide. Basic amino acids typically have positively charged side chains at physiological pH due to association with hydronium ion. Genetically encoded basic amino acids include L-Arg (R) and L-Lys (K).

“Bicyclic ketone” refers to a compound having the bicyclo skeleton that features at least two joined rings and at least one carbonyl group. The bicyclic ketone is preferably a fused cycloalkyl or heterocyclyl compound, e.g., octahydronaphthalenone, e.g., octahydronaphthalen-1(2H)-one, hexahydropentalenone, e.g., hexahydropentalen-2(1H)-one, hexahydrocyclopenta[c]pyrrolone, e.g., hexahydrocyclopenta[c]pyrrol-5(1H)-one. Preferably, the bicyclic ketone substrate is achiral. More preferably, each ring member of the bicyclic ketone has the same number of ring atoms. The bicyclic ketone structure may be substituted by one or more substituents. The substituents can themselves be optionally substituted. The substituents may be bonded via a carbon atom or heteroatom, e.g., N. In an embodiment, the bicyclic ketone has between 6 and 12 members in total.

“Bicyclic secondary alcohol product” refers to the product produced as a result of the application of a KRED enzyme, e.g., as disclosed herein, to a bicyclic ketone substrate.

The terms (cis) and (trans) alcohol product refer to the stereochemical configuration of the hydroxyl group relative to the hydrogens at the ring junction of the fused bicyclic compound. The (cis) alcohol refers to a compound whereby the hydroxyl group is present on the same side (or face) as the hydrogens of the ring junction and the (trans) alcohol refers to a compound whereby the hydroxyl is present on the opposite side (or face) of the hydrogens at the ring junction of the fused bicyclic product.

“Chiral” refers to molecules which have the property of non-superimposability of the mirror image partner, while the term “achiral” refers to molecules which are superimposable on their mirror image partner.

“Coding sequence” refers to that portion of a nucleic acid (e.g., a gene) that encodes an amino acid sequence of a polypeptide.

“Codon optimized” refers to changes in the codons of a polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the ketoreductases enzymes herein may be codon optimized for optimal production from the host organism selected for expression.

The terms “preferred,” “optimal,” “high,” or “codon usage bias” when used in the context of codons are used interchangeably to indicate codons that are used at a higher frequency in the protein coding regions than other codons that code for the same amino acid. Preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; Codon W, John Peden, University of Nottingham; McInemey, J. 0, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

The terms “cofactor regeneration system” and “cofactor recycling system” may be used interchangeably and refer to a set of reactants that participate in a reaction that reduces the oxidized form of the cofactor (e.g., NADP⁺ to NADPH). Cofactors oxidized by the ketoreductase-catalyzed reduction of the keto substrate are regenerated in reduced form by the cofactor regeneration system. Cofactor regeneration systems comprise a stoichiometric reductant that is a source of reducing hydrogen equivalents and is capable of reducing the oxidized form of the cofactor. The cofactor regeneration system may further comprise a catalyst, for example an enzyme catalyst that catalyzes the reduction of the oxidized form of the cofactor by the reductant. Cofactor regeneration systems to regenerate NADH or NADPH from NAD⁺ or NADP⁺, respectively, are known in the art and may be used in the methods described herein.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20% or less as compared to the reference sequence (which does not comprise additions or deletions) for optimal alignment of the two sequences. The comparison window can be longer than 20 contiguous residues, and includes, optionally 30, 40, 50, 100, or longer windows.

“Conservative” amino acid substitutions or mutations refer to the interchangeability of residues having similar side chains, and thus typically involves substitution of the amino acid in the polypeptide with amino acids within the same or similar defined class of amino acids. However, as used herein, conservative mutations do not include substitutions from a hydrophilic to hydrophilic, hydrophobic to hydrophobic, hydroxyl-containing to hydroxyl-containing, or small to small residue, if the conservative mutation can instead be a substitution from an aliphatic to an aliphatic, non-polar to non-polar, polar to polar, acidic to acidic, basic to basic, aromatic to aromatic, or constrained to constrained residue. Further, as used herein, A, V, L, or I can be conservatively mutated to either another aliphatic residue or to another non-polar residue. Table 1 below shows exemplary conservative substitutions.

TABLE 1
Conservative Substitutions

| Residue | Possible Conservative Mutations |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| P. H | Other constrained (P, H) |
| N, Q, S, T | Other polar (N, Q, S, T) |
| Y, W, F | Other aromatic (Y, W, F) |
| C | None |

“Constrained amino acid or residue” refers to an amino acid or residue that has a constrained geometry. Herein, constrained residues include L-pro (P) and L-his (H). Histidine has a constrained geometry because it has a relatively small imidazole ring. Proline has a constrained geometry because it also has a five membered ring.

“Control sequence” is defined herein to include all components, which are necessary or advantageous for the expression of a polypeptide of the present disclosure. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Conversion” refers to the enzymatic reduction of the substrate to the corresponding product. “Percent conversion” refers to the percent of the substrate that is reduced to the product within a period of time under specified conditions. Thus, the “enzymatic activity” or “activity” of a ketoreductase polypeptide can be expressed as “percent conversion” of the substrate to the product.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Deletion” refers to a modification in a polypeptide or polynucleotide characterized by the removal of one or more amino acids from a reference (e.g., parental) polypeptide or one or more nucleic acids from a reference (e.g., parental) polynucleotide. Deletions can comprise removal of 1 or more, 2 or more, 5 or more, 10 or more, 15 or more, 20 or more, 30 or more, 40 or more, or 50 or more amino acids or nucleic acids. Deletions may also comprise removal of up to 10% of the total number of amino acids or nucleic acids, or up to 20% of the total number of amino acids or nucleic acids, making up the reference enzyme while retaining enzymatic activity and/or retaining the improved properties of an engineered ketoreductase enzyme. Deletions can be directed to the internal portions and/or terminal portions of the polypeptide or polynucleotide. In various embodiments, the deletion can comprise a continuous segment or can be discontinuous.

“Derived from” or “originated from” as used herein in the context of engineered ketoreductase enzymes, identifies the originating ketoreductase enzyme (e.g., a wild-type ketoreductase enzyme), and/or the gene encoding such ketoreductase enzyme, upon which the engineering was based. For example, the engineered ketoreductase enzyme of SEQ ID NO: 426 was obtained by artificially evolving, over multiple generations the gene encoding the L. kefir ketoreductase enzyme of SEQ ID NO: 492. Thus, this engineered ketoreductase enzyme is “derived from” or “originated from” the wild-type ketoreductase of SEQ ID NO: 492.

“Different from” or “differs from” with respect to a designated reference (e.g., parental) sequence refers to difference of a given polypeptide or polynucleotide sequence when aligned to a reference (e.g., parental) sequence. Generally, the differences can be determined when the two sequences are optimally aligned. Differences include insertions, deletions, or substitutions of amino acid or nucleic acid residues in comparison to the reference sequence.

“Engineered ketoreductase” as used herein refers to a ketoreductase having a variant sequence generated by human manipulation (e.g, a sequence generated by directed evolution of a naturally occurring parent enzyme or directed evolution of a variant previously derived from a naturally occurring enzyme).

By “fragment” is meant a portion of a polypeptide or nucleic acid molecule. This portion contains, preferably, at least 10%, 20%, 30%, 40%, 50%, 60%, 70%, 80%, or 90% of the full-length of the reference nucleic acid molecule or polypeptide. A fragment may contain at least 10, 20, 30, 40, 50, 60, 70, 80, 90, 100, 200, 300, 400, 500, 600, 700, 800, 900, 1000, or more nucleotides or amino acids. In some embodiments, the fragment has an amino-terminal and/or carboxy-terminal deletion, but the remaining amino acid sequence is identical to the corresponding positions in the reference sequence.

“Heterologous polynucleotide” refers to any polynucleotide that is introduced into a host cell by laboratory techniques, and includes polynucleotides that are removed from a host cell, subjected to laboratory manipulation, and then reintroduced into a host cell.

“Hydrophilic Amino Acid or Residue” refers to an amino acid or residue having a side chain exhibiting a hydrophobicity of less than zero according to the normalized consensus hydrophobicity scale of Eisenberg et al., 1984, J. Mal. Biol. 179:125-142. Genetically encoded hydrophilic amino acids include L-Thr (T), L-Ser (S), L-His (H), L-Glu (E), L-Asn (N), L-Gln (Q), L-Asp (D), L-Lys (K) and L-Arg (R).

“Hydrophobic Amino Acid or Residue” refers to an amino acid or residue having a side chain exhibiting a hydrophobicity of greater than zero according to the normalized consensus hydrophobicity scale of Eisenberg et al., 1984, J. Mal. Biol. 179:125-142. Genetically encoded hydrophobic amino acids include L-Pro (P), L-Ile (I), L-Phe (F), L-Val (V), L-Leu (L), L-Trp (W), L-Met (M), L-Ala (A) and L-Tyr (Y).

“Hydroxyl-containing Amino Acid or Residue” refers to an amino acid containing a hydroxyl (—OH) moiety. Genetically-encoded hydroxyl-containing amino acids include L-Ser (S) L-Thr (T) and L-Tyr (Y).

“Improved enzyme property” refers to a ketoreductase polypeptide that exhibits an improvement in any enzyme property as compared to a reference (e.g., parental) ketoreductase. For the engineered ketoreductase polypeptides described herein, the comparison is generally made to a wild-type ketoreductase enzyme (e.g., L. kefir), although in some embodiments, the reference (e.g., parental) ketoreductase can be another improved engineered ketoreductase (e.g., SEQ ID NO: 54). Enzyme properties for which improvement is desirable include, but are not limited to, enzymatic activity (which can be expressed in terms of percent conversion of the substrate), thermal stability, solvent stability, pH activity profile, cofactor requirements, refractoriness to inhibitors (e.g., product inhibition), stereospecificity (including diastereospecificity or enantiospecificity), and stereoselectivity (including diastereoselectivity or enantioselectivity). In some embodiments, an engineered ketoreductase polypeptide exhibits an increased enzymatic activity (e.g., increased % conversion). In some embodiments, an engineered ketoreductase polypeptide exhibits reversed stereoselectivity (e.g., reversed enantioselectivity or reversed diastereoselectivity). In some embodiments, the increase in enzyme activity is an increase in the fold improvement over positive control (FIOP) % conversion or an increase in the fold improvement over positive control (FIOP) of desired product. In some embodiments, the improved enzyme property is an increase in selectivity (e.g., increase in desired product). In some embodiments, the increase in selectivity is an increase in the percent of the desired product or an increase in the FIOP % of desired product.

“Increased enzymatic activity” refers to an improved property of the engineered ketoreductase polypeptides, which can be represented by an increase in specific activity (e.g., product produced/time/weight protein) or an increase in percent conversion of the substrate to the product (e.g., percent conversion of starting amount of substrate to product in a specified time period using a specified amount of KRED) as compared to the reference (e.g., parental) ketoreductase enzyme. Exemplary methods to determine enzyme activity are provided in the Examples. Any property relating to enzyme activity may be affected, including the classical enzyme properties of Km, Vmax or kcat, changes of which can lead to increased enzymatic activity. Improvements in enzyme activity can be from about 1.5 times the enzymatic activity of the corresponding wild-type ketoreductase enzyme, to as much as 2 times, 5 times, 10 times, 20 times, 25 times, 50 times, 75 times, 100 times, 150 times, 200 times, 500 times, 1000, times, 3000 times, 5000 times, 7000 times or more enzymatic activity than the naturally occurring ketoreductase or another engineered ketoreductase from which the ketoreductase polypeptides were derived. In specific embodiments, the engineered ketoreductase enzyme exhibits improved enzymatic activity in the range of 150 to 3000 times, 3000 to 7000 times, or more than 7000 times greater than that of the parent ketoreductase enzyme. It is understood by the skilled artisan that the activity of any enzyme is diffusion limited such that the catalytic turnover rate cannot exceed the diffusion rate of the substrate, including any required cofactors. The theoretical maximum of the diffusion limit, or kca/Km, is generally about 108 to 109 (M-1 s-1). Hence, any improvements in the enzyme activity of the ketoreductase will have an upper limit related to the diffusion rate of the substrates acted on by the ketoreductase enzyme. Ketoreductase activity can be measured by any one of standard assays used for measuring ketoreductase, such as a decrease in absorbance or fluorescence of NADPH due to its oxidation with the concomitant reduction of a ketone to an alcohol, or by product produced in a coupled assay. Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

“Insertion” refers to modification to the polypeptide by addition of one or more amino acids from the reference (e.g., parental) polypeptide. In some embodiments, the improved engineered ketoreductase enzymes comprise insertions of one or more amino acids to the naturally occurring ketoreductase polypeptide as well as insertions of one or more amino acids to other improved ketoreductase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

The terms “isolated” or “purified” refer to material that is free to varying degrees from components which normally accompany it as found in its native state. “Isolate” denotes a degree of separation from original source or surroundings. “Purify” denotes a degree of separation that is higher than isolation. A “purified” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. “Substantially purified” refers to a composition in which the polynucleotide or polypeptide species is the predominant species present (i.e., on a molar or weight basis it is more abundant than any other individual macromolecular species in the composition), and is generally a substantially purified composition when the object species comprises at least about 50% of the macromolecular species present by mole or % weight. Generally, a substantially pure ketoreductase composition will comprise about 60%, 70%, 80%, 90%, 95%, 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated ketoreductase polynucleotides or polypeptides are a substantially pure polynucleotide or polypeptide composition.

By “isolated polynucleotide” is meant a nucleic acid that is free of the genes which, in the naturally occurring genome of the organism from which the nucleic acid molecule of the invention is derived, flank the gene. The term therefore includes, for example, a recombinant polynucleotide that is incorporated into a vector or that exists as a separate molecule independent of other sequences. In some embodiments, any of the polynucleotides encoding an engineered ketoreductase polypeptide disclosed herein are isolated polynucleotides.

By an “isolated polypeptide” is meant a polypeptide of the invention that has been separated from components or other contaminants that naturally accompany it, e.g., protein, lipids, and polynucleotides. Typically, the polypeptide is isolated when it is at least 60%, at least 75%, at least 90%, or at least 99%, by weight, free from the proteins and naturally occurring organic molecules with which it is naturally associated. Purity can be measured by any appropriate method, for example, column chromatography, polyacrylamide gel electrophoresis, or by HPLC analysis. The term embraces polypeptides which have been removed or purified from their naturally occurring environment or expression system (e.g., host cell or in vitro synthesis); or by chemically synthesizing the polypeptide. The ketoreductase enzymes disclosed herein may be present within a cell, present in the cellular medium, or prepared in various forms, such as lysates or isolated preparations. As such, in some embodiments, the engineered ketoreductase polypeptides disclosed herein are isolated polynucleotides.

“Ketoreductase” and “KRED” are used interchangeably herein to refer to a polypeptide having an enzymatic capability of reducing a carbonyl group to its corresponding alcohol. In some embodiments, a ketoreductase enzyme reduces a substrate to a (trans) alcohol product. For example, ketoreductase polypeptide may be capable of reducing tert-butyl rel-(3aR,6aS)-5-oxohexahydrocyclopenta[c]pyrrole-2(1H)-carboxylate (Compound 1 or Substrate) to tert-butyl rel-(3aR,5r,6aS)-5-hydroxyhexahydrocyclopenta[c]pyrrole-2(1H)-carboxylate ((5r)-2 or Product). In some embodiments, a ketoreductase enzyme reduces a substrate to a (cis) alcohol product. In a particular embodiment, the ketoreductase enzyme reduces a bicyclic ketone substrate to an alcohol product whereby the hydroxyl group is cis relative to the substituents bonded at the ring junction of the bicyclic ketone substrate. For example, as disclosed herein, the engineered ketoreductase polypeptides of the invention are capable of reducing tert-butyl rel-(3aR,6aS)-5-oxohexahydrocyclopenta[c]pyrrole-2(1H)-carboxylate (Compound 1 or Substrate) to tert-butyl rel-(3aR,5s,6aS)-5-hydroxyhexahydrocyclopenta[c]pyrrole-2(1H)-carboxylate ((5s)-2) or Product). A ketoreductase polypeptide typically utilizes a cofactor, e.g., reduced nicotinamide adenine dinucleotide (NADH) or reduced nicotinamide adenine dinucleotide phosphate (NADPH), as the reducing agent. “Ketoreductases” as used herein include naturally occurring (wild-type) ketoreductases (e.g., L. kefir) as well as non-naturally occurring engineered ketoreductase polypeptides (e.g., SEQ ID NO: 256) generated by human manipulation.

As used herein, the term amine protecting group (PG) in a compound of the disclosure refers to a group that should protect the amino functional groups concerned against unwanted secondary reactions, such as acylations, etherifications, esterifications, oxidations, solvolysis and similar reactions. It may be removed under deprotection conditions. Depending on the protecting group employed, the skilled person would know how to remove the protecting group to obtain the free amine NH₂ or NH group by reference to known procedures. These include reference to organic chemistry textbooks and literature procedures such as J. F. W. McOmie, “Protective Groups in Organic Chemistry”, Plenum Press, London and New York 1973; T. W. Greene and P. G. M. Wuts, “Greene's Protective Groups in Organic Synthesis”, Fourth Edition, Wiley, New York 2007; in “The Peptides”; Volume 3 (editors: E. Gross and J. Meienhofer), Academic Press, London and New York 1981; P. J. Kocienski, “Protecting Groups”, Third Edition, Georg Thieme Verlag, Stuttgart and New York 2005; and in “Methoden der organischen Chemie” (Methods of Organic Chemistry), Houben Weyl, 4th edition, Volume 15/I, Georg Thieme Verlag, Stuttgart 1974.

Preferred amine protecting groups in compounds of the disclosure generally comprise: C₁-C₆alkyl (e.g., tert-butyl), preferably C₁-C₄alkyl, more preferably C₁-C₂alkyl, most preferably C₁alkyl which is mono-, di- or tri-substituted by trialkylsilyl-C₁-C₇alkoxy (e.g., trimethylsilyethoxy), aryl, preferably phenyl, or a heterocyclic group (e.g., benzyl, cumyl, benzhydryl, pyrrolidinyl, trityl, pyrrolidinylmethyl, 1-methyl-1,1-dimethylbenzyl, (phenyl)methylbenzene) wherein the aryl ring or the heterocyclic group is unsubstituted or substituted by one or more, e.g., two or three, residues, e.g., selected from the group consisting of C₁-C₇alkyl, hydroxy, C₁-C₇alkoxy (e.g., para-methoxy benzyl (PMB)), C₂-C₈-alkanoyl-oxy, halogen, nitro, cyano, and CF₃, aryl-C₁-C₂-alkoxycarbonyl (preferably phenyl-C₁-C₂-alkoxycarbonyl (e.g., benzyloxycarbonyl (Cbz), benzyloxymethyl (BOM), pivaloyloxymethyl (POM)), C₁-C₁₀-alkenyloxycarbonyl, C₁-C₆alkylcarbonyl (e.g., acetyl or pivaloyl), C₆-C₁₀-arylcarbonyl; C₁-C₆-alkoxycarbonyl (e.g., tert-butyloxycarbonyl (Boc), methylcarbonyl, trichloroethoxycarbonyl (Troc), pivaloyl (Piv), allyloxycarbonyl), C₆-C₁₀-arylC₁-C₆-alkoxycarbonyl (e.g., 9-fluorenylmethyloxycarbonyl (Fmoc)), allyl or cinnamyl, sulfonyl or sulfenyl, succinimidyl group, silyl groups (e.g., triarylsilyl, trialkylsilyl, triethylsilyl (TES), trimethylsilylethoxymethyl (SEM), trimethylsilyl (TMS), triisopropylsilyl or tertbutyldimethylsilyl).

According to the disclosure, the preferred amine protecting group (PG) can be selected from the group comprising tert-butyloxycarbonyl (Boc), benzyloxycarbonyl (Cbz), para-methoxy benzyl (PMB), 2,4-dimethoxybenzyl (DMB), methyloxycarbonyl, trimethylsilylethoxymethyl (SEM) and benzyl. The amine protecting group (PG) is preferably an acid labile protecting group (can be removed in the presence of an acid, such as HCl, TFA), e.g., tert-butyloxycarbonyl (Boc), 2,4-dimethoxybenzyl (DMB), benzyloxycarbonyl (Cbz).

The term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

In the groups, radicals, or moieties defined below, the number of carbon atoms is often specified preceding the group, for example, C₁-C₈alkyl means an alkyl group or radical having 1 to 8 carbon atoms. In general, for groups comprising two or more subgroups, the last named group is the radical attachment point, for example, “alkylaryl” means a monovalent radical of the formula alkyl-aryl-, while “arylalkyl” means a monovalent radical of the formula aryl-alkyl-.

The term “halogen” or “halo” means fluorine, chlorine, bromine or iodine.

The term “alkyl” as used herein represents a saturated, branched or straight hydrocarbon group, e.g., having from 1 to 20 carbon atoms, e.g., C₁-C₃ alkyl, C₁-C₆ alkyl, C₂-C₈-alkyl, C₃-C₈-alkyl, C₁-C₈-alkyl, C₁-C₁₀ alkyl, C₁-C₂₀alkyl, and the like. Representative examples are methyl, ethyl, propyl (e.g., prop-1-yl, prop-2-yl (or iso-propyl)), butyl (e.g., 2-methylprop-2-yl (or tert-butyl), but-1-yl, but-2-yl), pentyl (e.g., pent-1-yl, pent-2-yl, pent-3-yl), 2-methylbut-1-yl, 3-methylbut-1-yl, hexyl (e.g., hex-1-yl), heptyl (e.g., hept-1-yl), octyl (e.g., oct-1-yl), nonyl (e.g., non-1-yl), and the like.

The term “alkenyl” as used herein represents a branched or straight hydrocarbon group having at least one double bond, e.g., having from respectively 2 to 20 carbon atoms and at least one double bond, e.g., C₂-C₃alkenyl, C₂-C₆ alkenyl, C₂-C₇ alkenyl, C₂-C₅ alkenyl, C₃-C₅ alkenyl, C₁-C₁₀-alkenyl, C₁-C₂₀alkenyl, and the like. Representative examples are ethenyl (or vinyl), propenyl (e.g., prop-1-enyl, prop-2-enyl), butadienyl (e.g., buta-1,3-dienyl), butenyl (e.g., but-1-en-1-yl, but-2-en-1-yl), pentenyl (e.g., pent-1-en-1-yl, pent-2-en-2-yl), hexenyl (e.g., hex-1-en-2-yl, hex-2-en-1-yl), 1-ethylprop-2-enyl, 1,1-(dimethyl)prop-2-enyl, 1-ethylbut-3-enyl, 1,1-(dimethyl)but-2-enyl, and the like.

The term “alkynyl” as used herein represents a branched or straight hydrocarbon group having at least one triple bond, e.g., having from respectively 2 to 20 carbon atoms and at least one triple bond, e.g., C₂-C₃ alkynyl, C₂-C₆ alkynyl, C₂-C₇ alkynyl, C₂-C₅ alkynyl, C₃-C₅ alkynyl, C₁-C₁₀ alkynyl, C₁-C₂₀alkynyl, and the like. Representative examples are ethynyl, propynyl (e.g., prop-1-ynyl, prop-2-ynyl), butynyl (e.g., but-1-ynyl, but-2-ynyl), pentynyl (e.g., pent-1-ynyl, pent-2-ynyl), hexynyl (e.g., hex-1-ynyl, hex-2-ynyl), 1-ethylprop-2-ynyl, 1,1-(dimethyl)prop-2-ynyl, 1-ethylbut-3-ynyl, 1,1-(dimethyl)but-2-ynyl, and the like.

The term “aryl” as used herein is intended to include monocyclic, bicyclic or polycyclic carbocyclic aromatic rings. Representative examples are phenyl, naphthyl (e.g., naphth-1-yl, naphth-2-yl), anthryl (e.g., anthr-1-yl, anthr-9-yl), phenanthryl (e.g., phenanthr-1-yl, phenanthr-9-yl), and the like. Aryl is also intended to include monocyclic, bicyclic or polycyclic carbocyclic aromatic rings substituted with carbocyclic aromatic rings. Representative examples are biphenyl (e.g., biphenyl-2-yl, biphenyl-3-yl, biphenyl-4-yl), phenylnaphthyl (e.g., 1-phenylnaphth-2-yl, 2-phenylnaphth-1-yl), and the like. Aryl is also intended to include partially saturated bicyclic or polycyclic carbocyclic rings with at least one unsaturated moiety (e.g., a benzo moiety). Representative examples are, indanyl (e.g., indan-1-yl, indan-5-yl), indenyl) (e.g., inden-1-yl, inden-5-yl), 1,2,3,4-tetrahydronaphthyl (e.g., 1,2,3,4-tetrahydronaphth-1-yl, 1,2,3,4-tetrahydronaphth-2-yl, 1,2,3,4-tetrahydronaphth-6-yl), 1,2-dihydronaphthyl (e.g., 1,2-dihydronaphth-1-yl, 1,2-dihydronaphth-4-yl, 1,2-dihydronaphth-6-yl), fluorenyl (e.g., fluoren-1-yl, fluoren-4-yl, fluoren-9-yl), and the like. Aryl is also intended to include partially saturated bicyclic or polycyclic carbocyclic aromatic rings containing one or two bridges. Representative examples are, benzonorbornyl (e.g., benzonorborn-3-yl, benzonorborn-6-yl), 1,4-ethano-1,2,3,4-tetrahydronapthyl (e.g., 1,4-ethano-1,2,3,4-tetrahydronapth-2-yl, 1,4-ethano-1,2,3,4-tetrahydronapth-10-yl), and the like. Aryl is also intended to include partially saturated bicyclic or polycyclic carbocyclic aromatic rings containing one or more spiro atoms. Representative examples are spiro[cyclopentane-1,1′-indane]-4-yl, spiro[cyclopentane-1,1′-indene]-4-yl, spiro[piperidine-4,1′-indane]-1-yl, spiro[piperidine-3,2′-indane]-1-yl, spiro[piperidine-4,2′-indane]-1-yl, spiro[piperidine-4,1′-indane]-3′-yl, spiro[pyrrolidine-3,2′-indane]-1-yl, spiro[pyrrolidine-3,1′-(3′,4′-dihydronaphthalene)]-1-yl, spiro[piperidine-3,1′-(3′,4′-dihydronaphthalene)]-1-yl, spiro[piperidine-4,1′-(3′,4′-dihydronaphthalene)]-1-yl, spiro[imidazolidine-4,2′-indane]-1-yl, spiro[piperidine-4,1′-indene]-1-yl, and the like.

The term C₆-C₁₄ aryl is to be interpreted accordingly.

Preferably, aryl refers to a monocyclic or bicyclic carbocyclic aromatic ring.

Preferred examples of aryl include, but are not limited to, phenyl and naphthyl. In an embodiment, aryl is phenyl.

As used herein, the term “heteroaryl” as used herein is intended to include monocyclic heterocyclic aromatic rings containing one or more heteroatoms selected from oxygen, nitrogen, and sulfur (O, N, and S). Representative examples are pyrrolyl, furanyl, thienyl, oxazolyl, thiazolyl, imidazolyl, pyrazolyl, isothiazolyl, isooxazolyl, triazolyl, (e.g., 1,2,4-triazolyl), oxadiazolyl, (e.g., 1,2,3-oxadiazolyl, 1,2,4-oxadiazolyl, 1,2,5-oxadiazolyl, 1,3,4-oxadiazolyl), thiadiazolyl (e.g., 1,2,3-thiadiazolyl, 1,2,4-thiadiazolyl, 1,2,5-thiadiazolyl, 1,3,4-thiadiazolyl), tetrazolyl, pyranyl, pyridinyl, pyridazinyl, pyrimidinyl, pyrazinyl, 1,2,3-triazinyl, 1,2,4-triazinyl, 1,3,5-triazinyl, thiadiazinyl, azepinyl, azecinyl, and the like.

Heteroaryl is also intended to include bicyclic heterocyclic aromatic rings containing one or more heteroatoms selected from oxygen, nitrogen, and sulfur (O, N, and S). Representative examples are indolyl, isoindolyl, benzofuranyl, benzothiophenyl, indazolyl, benzopyranyl, benzimidazolyl, benzothiazolyl, benzisothiazolyl, benzoxazolyl, benzisoxazolyl, benzoxazinyl, benzotriazolyl, naphthyridinyl, phthalazinyl, pteridinyl, purinyl, quinazolinyl, cinnolinyl, quinolinyl, isoquinolinyl, quinoxalinyl, oxazolopyridinyl, isooxazolopyridinyl, pyrrolopyridinyl, furopyridinyl, thienopyridinyl, imidazopyridinyl, imidazopyrimidinyl, pyrazolopyridinyl, pyrazolopyrimidinyl, pyrazolotriazinyl, thiazolopyridinyl, thiazolopyrimidinyl, imdazothiazolyl, triazolopyridinyl, triazolopyrimidinyl, and the like.

Heteroaryl is also intended to include polycyclic heterocyclic aromatic rings containing one or more heteroatoms selected from oxygen, nitrogen, and sulfur (O, N, and S). Representative examples are carbazolyl, phenoxazinyl, phenazinyl, acridinyl, phenothiazinyl, carbolinyl, phenanthrolinyl, and the like.

Heteroaryl is also intended to include partially saturated monocyclic, bicyclic or polycyclic heterocyclyls containing one or more heteroatoms selected oxygen, nitrogen, and sulfur (O, N, and S). Representative examples are imidazolinyl, indolinyl, dihydrobenzofuranyl, dihydrobenzothienyl, dihydrobenzopyranyl, dihydropyridooxazinyl, dihydrobenzodioxinyl (e.g., 2,3-dihydrobenzo[b][1,4]dioxinyl), benzodioxolyl (e.g., benzo[d][1,3]dioxole), dihydrobenzooxazinyl (e.g., 3,4-dihydro-2H-benzo[b][1,4]oxazine), tetrahydroindazolyl, tetrahydrobenzimidazolyl, tetrahydroimidazo[4,5-c]pyridyl, tetrahydroquinolinyl, tetrahydroisoquinolinyl, tetrahydroquinoxalinyl, and the like.

The heteroaryl ring structure may be substituted by one or more substituents. The substituents can themselves be optionally substituted. The heteroaryl ring may be bonded via a carbon atom or heteroatom.

The term “5-20 membered heteroaryl” is to be construed accordingly.

The term “monocyclic heteroaryl” as used herein is intended to include monocyclic heterocyclic aromatic rings as defined above.

The term “bicyclic heteroaryl” as used herein is intended to include bicyclic heterocyclic aromatic rings as defined above.

Examples of 5-20 membered heteroaryl include, but are not limited to, indolyl, imidazopyridyl, isoquinolinyl, benzooxazolonyl, pyridinyl, pyrimidinyl, pyridinonyl, benzotriazolyl, pyridazinyl, pyrazolotriazinyl, indazolyl, benzimidazolyl, quinolinyl, triazolyl, (e.g., 1,2,4-triazolyl), pyrazolyl, thiazolyl, oxazolyl, isooxazolyl, pyrrolyl, oxadiazolyl, (e.g., 1,2,3-oxadiazolyl, 1,2,4-oxadiazolyl, 1,2,5-oxadiazolyl, 1,3,4-oxadiazolyl), imidazolyl, pyrrolopyridinyl, tetrahydroindazolyl, quinoxalinyl, thiadiazolyl (e.g., 1,2,3-thiadiazolyl, 1,2,4-thiadiazolyl, 1,2,5-thiadiazolyl, 1,3,4-thiadiazolyl), pyrazinyl, oxazolopyridinyl, pyrazolopyrimidinyl, benzoxazolyl, indolinyl, isooxazolopyridinyl, dihydropyridooxazinyl, tetrazolyl, dihydrobenzodioxinyl (e.g., 2,3-dihydrobenzo[b][1,4]dioxinyl), benzodioxolyl (e.g., benzo[d][1,3]dioxole) and dihydrobenzooxazinyl (e.g., 3,4-dihydro-2H-benzo[b][1,4]oxazine).

The term “heterocyclyl” as used herein represents a saturated or partially saturated monocyclic or polycyclic ring containing one or more heteroatoms selected from nitrogen, oxygen, sulfur, S(═O) and S(═O)₂, and wherein there are no delocalized pi electrons (aromaticity) shared among the ring carbon or heteroatoms. The heterocyclyl ring structure may be substituted by one or more substituents. The substituents can themselves be optionally substituted. The heterocyclyl may be bonded via a carbon atom or heteroatom. The term polycyclic encompasses bridged, fused and spirocyclic heterocyclyl.

Representative examples are aziridinyl (e.g., aziridin-1-yl), azetidinyl (e.g., azetidin-1-yl, azetidin-3-yl), oxetanyl, pyrrolidinyl (e.g., pyrrolidin-1-yl, pyrrolidin-2-yl, pyrrolidin-3-yl), imidazolidinyl (e.g., imidazolidin-1-yl, imidazolidin-2-yl, imidazolidin-4-yl), oxazolidinyl (e.g., oxazolidin-2-yl, oxazolidin-3-yl, oxazolidin-4-yl), thiazolidinyl (e.g., thiazolidin-2-yl, thiazolidin-3-yl, thiazolidin-4-yl), isothiazolidinyl, piperidinyl (e.g., piperidin-1-yl, piperidin-2-yl, piperidin-3-yl, piperidin-4-yl), homopiperidinyl (e.g., homopiperidin-1-yl, homopiperidin-2-yl, homopiperidin-3-yl, homopiperidin-4-yl), piperazinyl (e.g., piperazin-1-yl, piperazin-2-yl), morpholinyl (e.g., morpholin-2-yl, morpholin-3-yl, morpholin-4-yl), thiomorpholinyl (e.g., thiomorpholin-2-yl, thiomorpholin-3-yl, thiomorpholin-4-yl), 1-oxothiomorpholinyl, 1,1-dioxo-thiomorpholinyl, tetrahydrofuranyl (e.g., tetrahydrofuran-2-yl, tetrahydrofuran-3-yl), tetrahydrothienyl, tetrahydro-1,1-dioxothienyl, tetrahydropyranyl (e.g., 2-tetrahydropyranyl), tetrahydrothiopyranyl (e.g., 2-tetrahydrothiopyranyl), 1,4-dioxanyl, 1,3-dioxanyl, and the like. Heterocyclyl is also intended to represent a saturated 6 to 8 membered bicyclic ring containing one or more heteroatoms selected from nitrogen, oxygen, sulfur, S(═O) and S(═O)₂. Representative examples are octahydroindolyl (e.g., octahydroindol-1-yl, octahydroindol-2-yl, octahydroindol-3-yl, octahydroindol-5-yl), decahydroquinolinyl (e.g., decahydroquinolin-1-yl, decahydroquinolin-2-yl, decahydroquinolin-3-yl, decahydroquinolin-4-yl, decahydroquinolin-6-yl), decahydroquinoxalinyl (e.g., decahydroquinoxalin-1-yl, decahydroquinoxalin-2-yl, decahydroquinoxalin-6-yl) and the like. Heterocyclyl is also intended to represent a saturated 6 to 8 membered ring containing one or more heteroatoms selected from nitrogen, oxygen, sulfur, S(═O) and S(═O)₂ and having one or two bridges. Representative examples are 3-azabicyclo[3.2.2]nonyl, 2-azabicyclo[2.2.1]heptyl, 3-azabicyclo[3.1.0]hexyl, 2,5-diazabicyclo[2.2.1]heptyl, atropinyl, tropinyl, quinuclidinyl, 1,4-diazabicyclo[2.2.2]octanyl, and the like. Heterocyclyl is also intended to represent a 6 to 8 membered saturated ring containing one or more heteroatoms selected from nitrogen, oxygen, sulfur, S(═O) and S(═O)₂ and containing one or more spiro atoms. Representative examples are 1,4-dioxaspiro[4.5]decanyl (e.g., 1,4-dioxaspiro[4.5]decan-2-yl, 1,4-dioxaspiro[4.5]decan-7-yl), 1,4-dioxa-8-azaspiro[4.5]decanyl (e.g., 1,4-dioxa-8-azaspiro[4.5]decan-2-yl, 1,4-dioxa-8-azaspiro[4.5]decan-8-yl), 8-azaspiro[4.5]decanyl (e.g., 8-azaspiro[4.5]decan-1-yl, 8-azaspiro[4.5]decan-8-yl), 2-azaspiro[5.5]undecanyl (e.g., 2-azaspiro[5.5]undecan-2-yl), 2,8-diazaspiro[4.5]decanyl (e.g., 2,8-diazaspiro[4.5]decan-2-yl, 2,8-diazaspiro[4.5]decan-8-yl), 2,8-diazaspiro[5.5]undecanyl (e.g., 2,8-diazaspiro[5.5]undecan-2-yl), 1,3,8-triazaspiro[4.5]decanyl (e.g., 1,3,8-triazaspiro[4.5]decan-1-yl, 1,3,8-triazaspiro[4.5]decan-3-yl, 1,3,8-triazaspiro[4.5]decan-8-yl), and the like.

The terms “6- to 12-membered heterocyclyl” and “3- to 14-membered heterocyclyl” are to be construed accordingly.

As used herein, the term “cycloalkyl” means a monocyclic saturated or partially unsaturated carbon ring, e.g., containing 3-10 carbon atoms, wherein there are no delocalized pi electrons (aromaticity) shared among the ring carbon.

Representative examples are cyclopropenyl, cyclopropyl, cyclobutyl, cyclobutenyl, cyclopentyl, cyclohexyl, cycloheptanyl, cyclooctanyl, and the like.

The term “arylalkyl” (e.g., benzyl, phenylethyl, 3-phenylpropyl, 1-naphtylmethyl, 2-(1-naphtyl)ethyl and the like) represents an aryl group as defined above attached through an alkyl chain having the indicated number of carbon atoms or substituted alkyl group as defined above. The term C₇-C₂₀arylalkyl is to be construed accordingly.

As used herein, the term “optional” or “optionally substituted” means that the described event or circumstance may or may not occur; for example, “optionally substituted aryl” refers to an aryl group that may or may not be substituted. This description includes both substituted aryl groups and unsubstituted aryl groups.

Nonlimiting exemplary naturally occurring (wild-type) ketoreductase enzymes include those from Lactobacillus kefir (“L. kefir”), Lactobacillus brevis (“L. brevis”), or Lactobacillus minor (“L. minor”). In some embodiments, the naturally occurring (wild-type) ketoreductase is from L. kefir. An exemplary nucleic acid (Accession No. QGV24812) and amino acid sequence of a L. kefir ketoreductase (UniProKB Accession No. Q6WVP7) is provided below:

| >ENA\|QGV24812\|QGV24812.1 Lactobacillus kefir SDR family NAD(P)-dependent | |
|---|---|
| oxidoreductase | |
| (SEQ ID NO: 491) | |
| ATGACTGATCGTTTAAAAGGCAAAGTAGCAATTGTAACTGGCGGTACCTTGGGAATTGGCTT | |
| GGCAATCGCTGATAAGTTTGTTGAAGAAGGCGCAAAGGTTGTTATTACCGGCCGTCACGCTG | |
| ATGTAGGTGAAAAAGCTGCCAAATCAATCGGCGGCACAGACGTTATCCGTTTTGTCCAACAC | |
| GATGCTTCTGATGAAGCCGGCTGGACTAAGTTGTTTGATACGACTGAAGAAGCATTTGGCCC | |
| AGTTACCACGGTTGTCAACAATGCCGGAATTGCGGTCAGCAAGAGTGTTGAAGATACCACAA | |
| CTGAAGAATGGCGCAAGCTGCTCTCAGTTAACTTGGATGGTGTCTTCTTCGGTACCCGTCTT | |
| GGAATCCAACGTATGAAGAATAAAGGACTCGGAGCATCAATCATCAATATGTCATCTATCGA | |
| AGGTTTTGTTGGTGATCCAACTCTGGGTGCATACAACGCTTCAAAAGGTGCTGTCAGAATTA | |
| TGTCTAAATCAGCTGCCTTGGATTGCGCTTTGAAGGACTACGATGTTCGGGTTAACACTGTT | |
| CATCCAGGTTATATCAAGACACCATTGGTTGACGATCTTGAAGGGGCAGAAGAAATGATGTC | |
| ACAGCGGACCAAGACACCAATGGGTCATATCGGTGAACCTAACGATATCGCTTGGATCTGTG | |
| TTTACCTGGCATCTGACGAATCTAAATTTGCCACTGGTGCAGAATTCGTTGTCGATGGTGGA | |
| TACACTGCTCAATAA | |
| > UniProKB Accession No. Q6WVP7 | |
| (SEQ ID NO: 492) | |
| 1 | MTDRLKGKVA IVTGGTLGIG LAIADKFVEE GAKVVITGRH ADVGEKAAKS IGGTDVIRFV |
| 61 | QHDASDEAGW TKLEDTTEEA FGPVTTVVNN AGIAVSKSVE DTTTEEWRKL LSVNLDGVFF |
| 121 | GTRLGIQRMK NKGLGASIIN MSSIEGFVGD PTLGAYNASK GAVRIMSKSA ALDCALKDYD |
| 181 | VRVNTVHPGY IKTPLVDDLE GAEEMMSQRT KTPMGHIGEP NDIAWICVYL ASDESKFATG |
| 241 | AEFVVDGGYT AQ |

In some embodiments, the naturally occurring (wild-type) ketoreductase is from L. brevis. An exemplary amino acid sequence of a L. brevis ketoreductase (Genbank Accession No. CAD66648) is provided below:

```
> Genbank Accession No. CAD66648
(SEQ ID NO: 493)
    1 MSNRLDGKVA IITGGTLGIG LAIATKFVEE GAKVMITGRH SDVGEKAAKS VGTPDQIQFF
  61 QHDSSDEDGW TKLFDATEKA FGPVSTLVNN AGIAVNKSVE ETTTAEWRKL LAVNLDGVFF
121 GIRLGIQRMK NKGLGASIIN MSSIEGFVGD PSLGAYNASK GAVRIMSKSA ALDCALKDYD
181 VRVNTVHPGY IKTPLVDDLP GAEEAMSQRT KTPMGHIGEP NDIAYICVYL ASNESKFATG
241 SEFVVDGGYT AQ
```

In some embodiments, the naturally occurring (wild-type) ketoreductase is from L. minor. An exemplary amino acid sequence of a L. minor ketoreductase (U.S. Pat. Pub. No. 2004/0265978) is provided below:

```
> L. minor alcohol dehydrogenase
(SEQ ID NO: 494)
    1 MTDRLKGKVA IVTGGTLGIG LAIADKFVEE GAKVVITGRH ADVGEKAARS IGGTDVIRFV
  61 QHDASDETGW TKLEDTTEEA FGPVTTVVNN AGIAVSKSVE DTTTEEWRKL LSVNLDGVFF
121 GTRLGIQRMK NKGLGASIIN MSSIEGFVGD PALGAYNASK GAVRIMSKSA ALDCALKDYD
181 VRVNTVHPGY IKTPLVDDLE GAEEMMSQRT KTPMGHIGEP NDIAWICVYL ASDESKFATG
241 AEFVVDGGYT AQ
```

A non-naturally occurring engineered ketoreductase may include one or more amino acid differences as provided in Table 2, Table 4, Table 5, Table 6, or Table 7. Nonlimiting exemplary non-naturally occurring engineered ketoreductase polypeptides are provided in Table 4, Table 5, Table 6, or Table 7. Additional engineered ketoreductase polypeptides are described in International App. No. WO2010025085A2, which is hereby incorporated by reference herein.

“Naturally occurring” or “wild-type” refers to the form found in nature. For example, a naturally occurring or wild-type polypeptide or polynucleotide sequence is a sequence present in an organism that can be isolated from a source in nature and has not been intentionally modified by human manipulation. In some embodiments, the terms “naturally occurring” or “wild-type” refer to a ketoreductase enzyme polypeptide. In some embodiments, a naturally occurring (wild-type) ketoreductase enzyme is from L. kefir, L. brevis, or L. minor. In some embodiments, a naturally occurring (wild-type) ketoreductase enzyme is from L. kefir.

“Non-conservative substitution” refers to substitution or mutation of an amino acid in the polypeptide with an amino acid with significantly differing side chain properties. Non-conservative substitutions may use amino acids between, rather than within, the defined groups listed above. In one embodiment, a non-conservative mutation affects (a) the structure of the peptide backbone in the area of the substitution (e.g., proline for glycine) (b) the charge or hydrophobicity, or (c) the bulk of the side chain.

“Non-polar Amino Acid or Residue” refers to a hydrophobic amino acid or residue having a side chain that is uncharged at physiological pH and which has bonds in which the pair of electrons shared in common by two atoms is generally held equally by each of the two atoms (i.e., the side chain is not polar). Genetically encoded non-polar amino acids include L-Gly (G), L-Leu (L), L-Val (V), L-Ile (I), L-Met (M) and L-Ala (A).

“Operably linked” is defined herein as a configuration in which a control sequence is appropriately placed at a position relative to the coding sequence of the DNA sequence such that the control sequence directs the expression of a polynucleotide and/or polypeptide.

“Polar Amino Acid or Residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (Q), L-Ser (S) and L-Thr (T).

“pH stable” refers to a ketoreductase polypeptide that maintains similar activity (more than e.g., 60% to 80%) after exposure to high or low pH (e.g., 4.5-6 or 8 to 12) for a period of time (e.g., 0.5-24 hrs) compared to the untreated enzyme.

A “Promoter” is a nucleic acid sequence that is recognized by a host cell for expression of a coding region. A control sequence may comprise an appropriate promoter. The promoter contains transcriptional control sequences, which mediate the expression of the polypeptide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

The terms “recombinant” and “engineered” are used interchangeably herein and when used with reference to, e.g., a cell, polynucleotide, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant or engineered ketoreductase polypeptides or recombinant cells expressing genes that are not found within the native (non-recombinant) form of the cell or express native genes that are otherwise expressed at a different level.

By “reference” is meant a standard or control condition.

A “reference sequence” is a defined sequence, such as a polynucleotide or polypeptide sequence, used as a basis for sequence comparison. A reference sequence may be a subset of or the entirety of a specified sequence (e.g., a segment of a full-length gene or polypeptide sequence). Generally, a reference sequence is at least about 20 nucleotide or amino acid residues in length, at least 25 nucleotide or amino acid residues in length, at least 50 nucleotide or amino acid residues in length, or the full length of the nucleic acid or polypeptide, or any integer thereabout or therebetween. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptides are typically performed by comparing sequences of the two polynucleotides over a “comparison window” to identify and compare local regions of sequence similarity. In some embodiments, the reference sequence is the same as the parental sequence used to generate an engineered ketoreductase polynucleotide or polypeptide. In some embodiments, the reference sequence is a wild-type (e.g., L. kefir) ketoreductase polynucleotide or polypeptide. In some embodiments, the reference sequence is an engineered (e.g., SEQ ID NO: 256) ketoreductase polynucleotide or polypeptide.

As used herein, the terms “salt,” “salts” or “salt form” refer to an acid addition or base addition salt of a respective compound, e.g., the compounds specified herein (e.g., Compound (I) or further pharmaceutical active ingredient, for example, as defined herein). “Salts” include in particular “pharmaceutically acceptable salts.” The term “pharmaceutically acceptable salts” refers to salts that retain the biological effectiveness and properties of the compounds and, which typically are not biologically or otherwise undesirable. The compounds, as specified herein (e.g., Compound (I) or further pharmaceutical active ingredient, for example, as defined herein), may be capable of forming acid and/or base salts by virtue of the presence of amino and/or carboxyl groups or groups similar thereto. The compound of the invention is capable of forming acid addition salts, thus, as used herein, the term pharmaceutically acceptable salt of Compound (I) means a pharmaceutically acceptable acid addition salt of Compound (I).

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Pharmaceutically acceptable salts can be synthesized from a basic or acidic moiety, by conventional chemical methods. Generally, such salts can be prepared by reacting the free acid forms of the compound with a stoichiometric amount of the appropriate base (such as Na, Ca, Mg, or K hydroxide, carbonate, bicarbonate or the like), or by reacting the free base form of the compound with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences,” 22^{nd} edition, Mack Publishing Company (2013); and in “Handbook of Pharmaceutical Salts: Properties, Selection, and Use” by Stahl and Wermuth (Wiley-VCH, Weinheim, 2011, 2^{nd} edition).

“Small Amino Acid or Residue” refers to an amino acid or residue having a side chain that is composed of a total three or fewer carbon and/or heteroatoms (excluding the a-carbon and hydrogens). The small amino acids or residues may be further categorized as aliphatic, non-polar, polar or acidic small amino acids or residues, in accordance with the above definitions. Genetically-encoded small amino acids include L-Ala (A), L-Val (V), L-Cys (C), L-Asn (N), L-Ser (S), L-Thr (T) and L-Asp (D).

The small amino acid L-Cys (C) is unusual in that it can form disulfide bridges with other L-Cys (C) amino acids or other sulfanyl- or sulfuydryl-containing amino acids. Cysteine-like residues include cysteine and other amino acids that contain sulfuydryl moieties that are available for formation of disulfide bridges. The ability of L-Cys (C) (and other amino acids with —SH containing side chains) to exist in a peptide in either the reduced free —SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present disclosure L-Cys (C) is categorized into its own unique group.

“Sequence identity,” “percentage (%) identity” and “homology” are used interchangeably herein to refer to the similarity between amino acid or nucleic acid sequences, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods.

The percentage may be calculated by determining the number of positions at which the identical nucleic acid base or amino acid residue occurs in both sequences to yield the number of matched positions, dividing the number of matched positions by the total number of positions in the comparison window and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the comparison window and multiplying the result by 100 to yield the percentage of sequence identity.

Those of skill in the art appreciate that there are many established algorithms available to align two sequences. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, FASTA, TFASTA, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

In addition, other programs and alignment algorithms are described in, for example, Smith and Waterman, 1981, Adv. Appl. Math. 2:482; Needleman and Wunsch, 1970, J. Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129.

The NCBI Basic Local Alignment Search Tool (BLAST™) (Altschul et al. 1990, J. Mol. Biol. 215:403-410) is readily available from several sources, including the National Center for Biotechnology Information (NCBI, Bethesda, Md.) and on the Internet, for use in connection with the sequence analysis programs blastp, blastn, blastx, tblastn and tblastx. This algorithm involves first identifying high scoring sequence pairs (HSPs) by identifying short words of length W in the query sequence, which either match or satisfy some positive-valued threshold score T when aligned with a word of the same length in a database sequence. This is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >O) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys, Madison WI), using default parameters provided.

By “substantially identical” or “substantial identity” is meant a polypeptide or polynucleotide exhibiting at least 50% identity to a reference amino acid sequence (for example, any one of the amino acid sequences described herein) or nucleic acid sequence (for example, any one of the nucleic acid sequences described herein). In some embodiments, such a sequence is at least about 60%, 70%, 80%, 85%, 90%, 91%, 92%, 93%, 94%, 95%, 96%, 97%, 98% or 99% identical as compared to a reference sequence over a comparison window of at least 20 residues or more. Typically, the percentage of sequence identity is calculated by comparing the reference sequence to a sequence that includes deletions or additions that total 20% or less of the reference sequence over the comparison window. In specific embodiments, the term “substantial identity,” as applied to polypeptides, means that two polypeptide sequences when optimally aligned, such as by the programs GAP or BESTFIT using default gap weights, share at least about 80% sequence identity, at least about 85% sequence identity, at least about 90% sequence identity, at least about 95% sequence identity or more (e.g., 99% sequence identity). Polynucleotides useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity.

“Stereoisomers” refer to isomers that have the same molecular formula, but differ in the three-dimensional spatial arrangement of atoms. Two types of stereoisomers include “enantiomers” and “diastereomers.” “Enantiomers” refer to a pair of stereoisomer compounds with the same molecular formula, but are non-superimposable mirror images of each other, and typically have the same physical properties. “Diastereomers” refer to a pair of stereoisomer compounds with the same molecular formula, but are non-superimposable, non-mirror images of each other, and typically have different physical properties. Diastereomers are not considered to be enantiomers. As provided herein, the (trans) alcohol product tert-butyl rel-(3aR,5r,6aS)-5-hydroxyhexahydrocyclopenta[c]pyrrole-2(1H)-carboxylate ((5r)-2) and (cis) alcohol product tert-butyl rel-(3aR,5s,6aS)-5-hydroxyhexahydrocyclopenta[c]pyrrole-2(1H)-carboxylate ((5s)-2) are stereoisomers of one another. As shown in FIG. 1, the structures of (5r)-2 and (5s)-2 are non-superimposable, non-mirror images and are therefore also referred herein as diastereoisomers.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as “enantioselectivity,” the fraction (typically reported as a percentage) of one enantiomer in the sum of both. It is commonly alternatively reported in the art (typically as a percentage) as the enantiomeric excess (e.e.) calculated therefrom according to the formula [major enantiomer−minor enantiomer]/[major enantiomer+minor enantiomer]. Where the stereoisomers are diastereoisomers, the stereoselectivity is referred to as “diastereoselectivity,” the fraction (typically reported as a percentage) of one diastereomer in a mixture of two diastereomers, commonly alternatively reported as the diastereomeric excess (d.e.) calculated therefrom according to the formula [major diastereomer−minor diastereomer]/[major diastereomer+minor diastereomer]. Enantiomeric excess and diastereomeric excess are types of stereometric excess. “Highly stereoselective” refers to a ketoreductase polypeptide that is capable of converting or reducing the substrate to the corresponding (cis) alcohol product with at least about 99% diastereomeric excess.

“Stereospecificity” refers to the preferential conversion in a chemical or enzymatic reaction of one stereoisomer over another. Stereospecificity can be partial, where the conversion of one stereoisomer is favored over the other, or it may be complete where only one stereoisomer is converted. When the stereoisomers are enantiomers, the stereospecificity is referred to as “enantiospecificity.” Where the stereoisomers are diastereoisomers, the stereospecificity is referred to as “diastereospecificity.”

“Suitable reaction conditions” or “reaction conditions suitable for reducing or converting the substrate to the product compound” refer to those conditions (e.g., enzyme loading, substrate loading, cofactor loading, temperature, pH, buffer, co-solvent, etc.) in the biocatalytic reaction system, under which the KRED polypeptide of the present disclosure can convert a substrate to a desired product compound. Exemplary “suitable reaction conditions” are provided in the present disclosure and illustrated by the Examples.

“Reference to,” “relative to,” “compared to” or “corresponding to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered KRED, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Ketoreductase Enzymes

The present disclosure provides engineered ketoreductase (“KRED”) enzymes that are capable of stereoselectively reducing a defined bicyclic keto substrate to its corresponding alcohol product and having improved properties when compared with naturally occurring, wild-type KRED enzymes (e.g., wild-type L. kefir KRED enzymes) or engineered KRED variants thereof (e.g., SEQ ID NO: 54, 152, or 256). Naturally occurring, wild-type KRED enzymes (e.g., wild-type L. kefir ketoreductases) reduce the compound preferentially on one face of the keto-group. Specifically, when reducing the bicyclic ketone such as tert-butyl rel-(3aR,6aS)-5-oxohexahydrocyclopenta[c]pyrrole-2(1H)-carboxylate (C₁₂H₁₉NO₃ (MW: 225.29); referred to herein as Compound 1 or Substrate), wild-type KRED enzymes, such as from L. kefir, display a strong specificity toward the formation of tert-butyl rel-(3aR,5r,6aS)-5-hydroxyhexahydrocyclopenta[c]pyrrole-2(1H)-carboxylate (C₁₂H₂₁NO₃ (MW: 227.30); referred to herein as (5r)-2 or (trans) alcohol product).

However, the present disclosure provides engineered KRED enzyme polypeptides that are capable of reducing Compound 1 to tert-butyl rel-(3aR,5s,6aS)-5-hydroxyhexahydrocyclopenta[c]pyrrole-2(1H)-carboxylate (C₁₂H₂₁NO₃ (MW: 227.30); referred to herein as (5s)-2 or (cis) alcohol product) with very high selectivity and specificity (see FIG. 1). The present disclosure further provides polynucleotides encoding such engineered KRED polypeptides, methods for using and producing the engineered KRED polypeptides, and kits thereof.

In some embodiments, the engineered enzymes described herein have one or more improved properties. Improvements in enzyme property include, among others, increases or changes in enzyme activity, cofactor binding, stereoselectivity, stereospecificity, thermostability, solvent stability, or reduced product inhibition. In some embodiments, the improved enzyme property is reversed stereoselectivity (e.g., reversed enantioselectivity or reversed diastereoselectivity).

In some embodiments, the improved enzyme property is reversed or increased diastereoselectivity. In some embodiments, the improved enzyme property is an increase in enzymatic activity (e.g., increase in conversion rate). In some embodiments, the improved enzyme property is an increase in selectivity (e.g., increase in desired product or diastereomeric excess). In some embodiments, the improved enzyme property is the ability to use less cofactor in a reduction reaction. In some embodiments, the improved enzyme property is the ability to not require glucose dehydrogenase (GDH)/glucose cofactor recycling in a reduction reaction. In some embodiments, the improved enzyme property is the ability to not require dimethylsulfoxide (DMSO) in a reduction reaction.

Generally, the engineered KRED polypeptides have an improved property (e.g., reversed or increased diastereoselectivity (i.e., selectivity for (5s)-2 over (5r)-2) or activity (% conversion)) as compared to a naturally occurring wild-type ketoreductase enzyme (e.g., obtained from Lactobacillus kefir (“L. kefir”; SEQ ID NO: 492), Lactobacillus brevis (“L. brevis”; SEQ ID NO: 493), or Lactobacillus minor (“L. minor”; SEQ ID NO: 494)). In some embodiments, the engineered KREDs enzymes have an improved property (e.g., reversed or increased diastereoselectivity (i.e., selectivity for (5s)-2 over (5r)-2) or activity (% conversion)) as compared to a wild-type L. kefir ketoreductase (e.g., SEQ ID NO: 492). For example, in some embodiments, an engineered KRED polypeptide as described herein has increased enzymatic activity as compared to a wild-type KRED enzyme (e.g., L. kefir) for reducing the substrate (e.g., Compound 1) to the product (e.g., (5s)-2) and/or further reverses or increases diastereoselectivity for the (cis) alcohol product diastereomer.

In some embodiments, the engineered KRED polypeptides of the disclosure have one or more improved properties (e.g., reversed or increased diastereoselectivity (i.e., selectivity for (5s)-2 over (5r)-2) or activity (% conversion)) as compared to an engineered KRED polypeptide (e.g., SEQ ID NO: 54, 152, or 256) that is obtained or derived from a naturally occurring ketoreductase enzyme (e.g., L. kefir; SEQ ID NO: 492). In some embodiments, the engineered KRED polypeptides of the disclosure have an improved property (e.g., reversed or increased diastereoselectivity (i.e., selectivity for (5s)-2 over (5r)-2) or activity (% conversion)) as compared to commercially available KRED enzymes (e.g., ADH-152). In some embodiments, the engineered KRED polypeptides of the disclosure have an improved property (e.g., reversed or increased diastereoselectivity (i.e., selectivity for (5s)-2 over (5r)-2) or activity (% conversion)) as compared to an engineered KRED polypeptide selected from Table 4, Table 5, or Table 6. In some embodiments, the engineered KRED polypeptides of the disclosure have an improved property (e.g., reversed or increased diastereoselectivity (i.e., selectivity for (5s)-2 over (5r)-2) or activity (% conversion)) as compared the engineered KRED polypeptide of SEQ ID NO: 54, 152, or 256.

In some embodiments, the engineered KRED polypeptides of the invention have an improved property as compared to a reference (e.g., parental) polypeptide (e.g., SEQ ID NO: 54, 152, 256, or 492). In some embodiments, the reference polypeptide is a commercially available KRED enzyme (e.g., ADH-152). In some embodiments, the reference (e.g., parental) polypeptide is a wild-type KRED polypeptide. In some embodiments, the reference (e.g., parental) polypeptide is an engineered KRED variant (e.g., SEQ ID NO: 54, SEQ ID NO: 152, SEQ ID NO: 256). In some embodiments, the reference (e.g., parental) polypeptide is SEQ ID NO: 54, SEQ ID NO: 152, SEQ ID NO: 256, or SEQ ID NO: 492.

In some embodiments, the engineered KRED polypeptides of the invention are improved by having an increased level or rate of enzymatic activity as compared to a reference (e.g., parental) polypeptide (e.g., SEQ ID NO: 54, 152, 256, or 492). In some embodiments, the level of activity is measured with respect to their fold improvement over positive control (FIOP) (e.g., % conversion). In some embodiments, the engineered KRED polypeptides are capable of a FIOP that is greater than about 1.75, greater than about 2.00, greater than about 2.25, greater than about 2.50, greater than about 2.75, greater than about 3.00, greater than about 3.25, greater than about 3.50, greater than about 3.75, greater than about 4.00, greater than about 4.25, greater than about 4.50, greater than about 4.75, greater than about 5.00, greater than about 5.25, greater than about 5.50, greater than about 5.75, or greater than about 6.00, greater than about 6.25, or greater than about 6.50, as compared to a reference (e.g., parental) polypeptide as the positive control.

In some embodiments, the engineered KRED polypeptides of the invention are improved as compared to a reference (e.g., parental) polypeptide (e.g., SEQ ID NO: 54, 152, 256, or 492) with respect to their stereoselectivity (e.g., selectivity for (5s)-2 over (5r)-2). In particular, the engineered KRED polypeptides of the invention are improved as compared to a reference (e.g., parental) polypeptide (e.g., SEQ ID NO: 54, 152, 256, or 492) with respect to their diastereoselectivity (i.e., selectivity for (5s)-2 over (5r)-2). In some embodiments, the engineered KRED polypeptides of the invention are improved by having an increased level of selectivity as compared to a reference (e.g., parental) polypeptide (e.g., SEQ ID NO: 54, 152, 256, or 492) with respect to their fold improvement over positive control (FIOP) % of desired product (e.g., (5s)-2). In some embodiments, the engineered KRED polypeptides are capable of a FIOP (e.g., % of desired product (e.g., (5s)-2)) that is greater than about 1.10, greater than about 1.25, greater than about 1.50, greater than about 1.75, greater than about 2.00, greater than about 2.25, or greater than about 2.50 as compared to a reference (e.g., parental) polypeptide as the positive control.

In some embodiments, the engineered KRED polypeptides described herein have an amino acid sequence with one or more amino acid differences as compared to a reference (e.g., parental) amino acid sequence (e.g., wild-type KRED (e.g., L. kefir) or an engineered KRED (e.g., SEQ ID NO: 54)) that results in an improved property (e.g., reversed or increased diastereoselectivity (i.e., selectivity for (5s)-2 over (5r)-2) or activity (% conversion)) of the enzyme for a defined keto substrate. In some embodiments, the engineered KRED polypeptides described herein have an amino acid sequence with one or more amino acid differences as compared to wild-type KRED (e.g., L. kefir) that results in an improved property (e.g., reversed or increased diastereoselectivity (i.e., selectivity for (5s)-2 over (5r)-2) or activity (% conversion)) of the enzyme for a defined keto substrate. In some embodiments, the engineered KRED polypeptides described herein have an amino acid sequence with one or more amino acid differences as compared to an engineered KRED (e.g., SEQ ID NO: 54, 152, or 256)) that results in an improved property (e.g., reversed or increased diastereoselectivity (i.e., selectivity for (5s)-2 over (5r)-2) or activity (% conversion)) of the enzyme for a defined keto substrate.

In some embodiments, the engineered KRED polypeptides described herein may contain one or more of the observed mutation characteristics as provided in Table 2. In some embodiments, the engineered KRED polypeptides contain one or more amino acid differences as provided in Table 4, Table 5, Table 6, and Table 7. In some embodiments, the engineered KRED polypeptides contain one or more amino acid differences as provided in Table 2 and one or more amino acid differences as provided in Table 4, Table 5, Table 6, and Table 7.

TABLE 2
KRED Characteristics

| Position | 2 | 7 | 17 | 18 | 21 | 25 | 40 | 43 | 45 | 46 | 54 | 56 |
|---|---|---|---|---|---|---|---|---|---|---|---|---|
| L. kefir wt | T | G | L | G | L | D | H | V | E | K | T | V |
| (SEQ ID | | | | | | | | | | | | |
| NO: 492) | | | | | | | | | | | | |
| KRED | F | S | T | S | F | T | R | R | Q | R | R | S |
| Mutations | | R | R | R | S | | K | | L | | | D |
| observed | | K | G | | | | | | R | | | T |
| | | C | A | | | | | | H | | | |
| | | T | S | | | | | | | | | |
| | | | M | | | | | | | | | |
| | | | Q | | | | | | | | | |
| Position | 72 | 76 | 93 | 94 | 95 | 96 | 97 | 98 | 100 | 101 | 103 | 106 |
| L. kefir wt | K | T | I | A | V | S | K | S | E | D | T | E |
| (SEQ ID | | | | | | | | | | | | |
| NO: 492) | | | | | | | | | | | | |
| KRED | G | K | K | L | I | A | I | G | K | C | E | W |
| Mutations | T | | | W | M | G | M | C | A | F | W | L |
| observed | | | | V | P | Y | R | | R | Q | | V |
| | | | | I | | | | | | R | | I |
| | | | | | | | | | | N | | |
| | | | | | | | | | | S | | |
| | | | | | | | | | | T | | |
| | | | | | | | | | | G | | |
| | | | | | | | | | | V | | |
| | | | | | | | | | | L | | |
| | | | | | | | | | | A | | |
| Position | 108 | 113 | 117 | 135 | 145 | 147 | 151 | 152 | 155 | 170 | 173 | 176 |
| L. kefir wt | R | V | G | G | E | F | P | T | A | A | D | L |
| (SEQ ID | | | | | | | | | | | | |
| NO: 492) | | | | | | | | | | | | |
| KRED | H | I | S | A | S | V | S | Q | G | S | M | K |
| Mutations | F | | | | D | L | | L | | | W | |
| observed | | | | | F | | | R | | | V | |
| | | | | | | | | I | | | A | |
| | | | | | | | | K | | | L | |
| | | | | | | | | | | | R | |
| Position | 190 | 192 | 193 | 194 | 195 | 196 | 197 | 198 | 199 | 200 | 201 | 202 |
| L. kefir wt | Y | K | T | P | L | V | D | D | L | E | G | A |
| (SEQ ID | | | | | | | | | | | | |
| NO: 492) | | | | | | | | | | | | |
| KRED | A | H | R | C | M | L | M | Q | V | G | W | G |
| Mutations | P | R | G | L | | I | R | P | | W | | L |
| observed | V | D | Q | D | | P | | V | | | | |
| | C | M | | T | | G | | I | | | | |
| | | W | | E | | S | | Y | | | | |
| | | | | W | | A | | | | | | |
| | | | | V | | | | | | | | |
| | | | | A | | | | | | | | |
| | | | | S | | | | | | | | |

| Position | 205 | 206 | 208 | 211 | 219 | 220 | 223 |
|---|---|---|---|---|---|---|---|
| L. kefir wt | M | M | Q | K | E | P | I |
| (SEQ ID | | | | | | | |
| NO: 492) | | | | | | | |
| KRED | L | Q | A | L | L | T | V |
| Mutations | Y | A | T | V | | V | C |
| observed | F | L | V | | | | |
| | W | N | | | | | |
| | | W | | | | | |

In certain embodiments, the improved property (e.g., reversed or increased diastereoselectivity (i.e., selectivity for (5s)-2 over (5r)-2) or activity (% conversion)) is based on mutating one or more of the following residues: X17, X18, X40, X56, X94, X96, X106, X145, X173, X190, X194, X196, X198, X202, and/or X206 relative to the reference (e.g., parental) amino acid sequence (e.g., wild-type L. kefir ketoreductase or engineered variant thereof). In some embodiments, the improved property (e.g., reversed or increased diastereoselectivity (i.e., selectivity for (5s)-2 over (5r)-2) or activity (% conversion)) is based on mutating one or more of the following residues: X94, X96, X190, X196, X202, and/or X206 relative to the reference (e.g., parental) amino acid sequence (e.g., wild-type L. kefir ketoreductase or an engineered variant thereof). In some embodiments, the improved property (e.g., reversed or increased diastereoselectivity (i.e., selectivity for (5s)-2 over (5r)-2) or activity (% conversion)) is based on mutating one or more of the following residues: (i) X190 to a residue that is not tyrosine; (ii) X190 to a non-aromatic residue; (iii) X196 to an aliphatic residue or a small amino acid residue; (iv) X202 to a small amino acid residue; and/or (v) X206 to a residue that is not methionine, relative to the reference (e.g., parental) amino acid sequence (e.g., wild-type L. kefir ketoreductase or engineered variant thereof).

In some embodiments, the improved property (e.g., reversed or increased diastereoselectivity (i.e., selectivity for (5s)-2 over (5r)-2) or activity (% conversion)) is based on one or more of the following mutations: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198I, X198Y, X198D, X198P, X202A, X202G, X206Q, X206N and/or X206W relative to the reference (e.g., parental) amino acid sequence (e.g., wild-type L. kefir ketoreductase or engineered variant thereof). In some embodiments, the improved property (e.g., reversed or increased diastereoselectivity (i.e., selectivity for (5s)-2 over (5r)-2) or activity (% conversion)) is based on mutating one or more of the following residues: i) X17S, X17T, or X17G; ii) X18S or X18R; iii) X56S, X56T, or X56D; iv) X94I; v) X106I, X106V, X106L, or X106W; vi) X173A, X173W, X173M, or X173K; vii) X194A, X194V, X194W, or X194T; viii) X196G; and ix) X198V, X198I, X198Y, or X198P relative to the reference (e.g., parental) amino acid sequence (e.g., wild-type L. kefir ketoreductase or engineered variant thereof). In some embodiments, the improved property (e.g., reversed or increased diastereoselectivity (i.e., selectivity for (5s)-2 over (5r)-2) or activity (% conversion)) is based on one or more of the following mutations: X94I, X96Y, X190A, X196G, X202A, X202G and/or X206W relative to the reference (e.g., parental) amino acid sequence (e.g., wild-type L. kefir ketoreductase or engineered variant thereof). In some embodiments, the improved property (e.g., reversed or increased diastereoselectivity (i.e., selectivity for (5s)-2 over (5r)-2) or activity (% conversion)) is based on one or more of the following mutations: X18G, X40H, X56V, X94A, X106E, X145E, X145, X173D, X194P, X198D, X202A, and X206Q relative to the reference (e.g., parental) amino acid sequence (e.g., wild-type L. kefir ketoreductase or engineered variant thereof).

In some embodiments, the engineered KRED polypeptides of the present disclosure with improved properties (e.g., reversed or increased diastereoselectivity (i.e., selectivity for (5s)-2 over (5r)-2) or activity (% conversion)) are derived from a wild-type ketoreductase (e.g., L. kefir, L. brevis, or L. minor). In some embodiments, the engineered KRED polypeptides of the disclosure have an amino acid sequence with one or more amino acid differences as compared to a wild-type L. kefir KRED polypeptide (SEQ ID NO: 492). In some embodiments, the engineered KRED polypeptides of the present disclosure have an amino acid sequence with one or more amino acid differences as provided in Table 2, Table 4, Table 5, Table 6, and Table 7 as compared to an amino acid sequence having at least about 80%, 85%, 90%, 95%, 96%, 97%, 98%, 99% or 100% to SEQ ID NO: 492. In some embodiments, the engineered KRED polypeptides of the present disclosure have an amino acid sequence with one or more amino acid differences as provided in Table 2, Table 4, Table 5, Table 6, and Table 7 as compared to the amino acid sequence of SEQ ID NO: 492.

In some embodiments, the engineered KRED polypeptides of the present disclosure have improved properties (e.g., reversed diastereoselectivity) as compared to the wild-type KRED polypeptide of SEQ ID NO: 492. In certain embodiments, the improved properties (e.g., reversed or increased diastereoselectivity (i.e., selectivity for (5s)-2 over (5r)-2) or activity (% conversion)) of the engineered KRED polypeptides of the present disclosure are based on mutating one or more of the following residues: X17, X18, X40, X56, X94, X96, X106, X145, X173, X190, X194, X196, X198, X202, and/or X206 relative to an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99%, or is 100% identical to SEQ ID NO: 492. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on mutating one or more of the following residues: X94, X96, X190, X196, X202, and/or X206 relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 492. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on mutating one or more of the following residues: (i) X190 to a residue that is not tyrosine; (ii) X190 to a non-aromatic residue; (iii) X196 to an aliphatic residue or a small amino acid residue; (iv) X202 to a small amino acid residue; and/or (v) X206 to a residue that is not methionine, relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 492. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198I, X198Y, X198D, X198P, X202A, X202G, X206Q, X206N and/or X206W relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 492. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: i) X17S, X17T, or X17G; ii) X18S or X18R; iii) X56S, X56T, or X56D; iv) X94I; v) X106I, X106V, X106L, or X106W; vi) X173A, X173W, X173M, or X173K; vii) X194A, X194V, X194W, or X194T; viii) X196G; and ix) X198V, X198I, X198Y, or X198P relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 492. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: X94I, X96Y, X190A, X196G, X202A, X202G and/or X206W relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 492. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: X18G, X40H, X56V, X94A, X106E, X145E, X145, X173D, X194P, X198D, X202A, and X206Q relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 492.

In some embodiments, the engineered KRED polypeptides of the present disclosure with improved properties (e.g., reversed or increased diastereoselectivity (i.e., selectivity for (5s)-2 over (5r)-2) or activity (% conversion)) are derived from an engineered variant of a wild-type (e.g., L. kefir) ketoreductase (e.g., SEQ ID NO: 54, 152, 256). In some embodiments, the polypeptides of the present disclosure have an amino acid sequence with one or more amino acid differences as compared to an engineered variant of a wild-type L. kefir ketoreductase (e.g., SEQ ID NO: 54, 152, 256). In some embodiments, the engineered KRED polypeptides of the disclosure have an amino acid sequence with one or more amino acid differences as compared to an engineered variant comprising an amino acid sequence having at least about 80%, 85%, 90%, 95%, 96%, 97%, 98%, 99% or 100% sequence identity to an amino acid sequence in Table 4, Table 5, Table 6 or Table 7. In some embodiments, the engineered KRED polypeptides of the disclosure have an amino acid sequence with one or more amino acid differences as compared to an engineered variant comprising an amino acid sequence in Table 4, Table 5, Table 6 or Table 7. In some embodiments, the engineered KRED polypeptides of the disclosure have an amino acid sequence with one or more amino acid differences as compared to an engineered variant comprising an amino acid sequence having at least about 80%, 85%, 90%, 95%, 96%, 97%, 98%, 99% or 100% sequence identity to SEQ ID NO: 54, SEQ ID NO: 152, or SEQ ID NO: 256. In some embodiments, the engineered KRED polypeptides of the disclosure have an amino acid sequence with one or more amino acid differences as compared to an engineered variant comprising the amino acid sequence of SEQ ID NO: 54, SEQ ID NO: 152, or SEQ ID NO: 256.

In some embodiments, the engineered KRED polypeptides have an amino acid sequence with one or more amino acid differences selected from Table 2, Table 4, Table 5, Table 6, and Table 7 as compared to an engineered KRED polypeptide (e.g., SEQ ID NO: 2, SEQ ID NO: 54, SEQ ID NO: 152, or SEQ ID NO: 256). In some embodiments, the engineered KRED polypeptides have an amino acid sequence with one or more amino acid differences selected from Table 2 and one or more amino acid differences selected from Table 4, Table 5, Table 6, and Table 7 as compared to an engineered KRED polypeptide (e.g., SEQ ID NO: 2, SEQ ID NO: 54, SEQ ID NO: 152, or SEQ ID NO: 256). In some embodiments, the engineered KRED polypeptides of the present disclosure comprise one or more amino acid differences selected from Table 2, Table 4, Table 5, Table 6, and Table 7, relative to an amino acid sequence that is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 54, SEQ ID NO: 152, or SEQ ID NO: 256. In some embodiments, the engineered KRED polypeptides of the present disclosure comprise one or more amino acid differences selected from Table 2 and one or more amino acid differences selected from Table 4, Table 5, Table 6, or Table 7, relative to an amino acid sequence that is at least about 85%, 86%, 87%, 88%, 89%, 90%, 91%, 92%, 93%, 94%, 95%, 96%, 97%, 98%, 99% or 100% identical to SEQ ID NO: 54, SEQ ID NO: 152, or SEQ ID NO: 256. In some embodiments, the engineered KRED polypeptides of the present disclosure comprise one or more amino acid differences selected from Table 2, Table 4, Table 5, Table 6, or Table 7, relative to SEQ ID NO: 54, SEQ ID NO: 152, or SEQ ID NO: 256. In some embodiments, the engineered KRED polypeptides of the present disclosure comprise one or more amino acid differences selected from Table 2 and one or more amino acid differences selected from Table 4, Table 5, Table 6, or Table 7, relative to SEQ ID NO: 54, SEQ ID NO: 152, or SEQ ID NO: 256.

In some embodiments, the engineered KRED polypeptides of the present disclosure have improved properties as compared to the engineered KRED polypeptide of SEQ ID NO: 54. In some embodiments, the engineered KRED polypeptides of the present disclosure are capable of selectively reducing tert-butyl rel-(3aR,6aS)-5-oxohexahydrocyclopenta[c]pyrrole-2(1H)-carboxylate (Substrate) to tert-butyl rel-(3aR,5s,6aS)-5-hydroxyhexahydrocyclopenta[c]pyrrole-2(1H)-carboxylate (Product) under suitable reaction conditions at a greater stereoselectivity (% diastereomeric excess) and/or activity (% conversion) than that of SEQ ID NO: 54.

In certain embodiments, the improved properties (e.g., reversed or increased diastereoselectivity (i.e., selectivity for (5s)-2 over (5r)-2) or activity (% conversion)) of the engineered KRED polypeptides of the present disclosure are based on mutating one or more of the following residues: X17, X18, X40, X56, X94, X96, X106, X145, X173, X190, X194, X196, X198, X202, and/or X206 relative to an amino acid sequence that is at least about 85%, 86%, 87%, 88%, 89%, 90%, 91%, 92%, 93%, 94%, 95%, 96%, 97%, 98%, or 99%, or is 100% identical to SEQ ID NO: 54. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on mutating one or more of the following residues: X94, X96, X190, X196, X202, and/or X206 relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 54. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on mutating one or more of the following residues: (i) X190 to a residue that is not tyrosine; (ii) X190 to a non-aromatic residue; (iii) X196 to an aliphatic residue or a small amino acid residue; (iv) X202 to a small amino acid residue; and/or (v) X206 to a residue that is not methionine, relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 54. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198I, X198Y, X198D, X198P, X202A, X202G, X206Q, X206N and/or X206W relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 54. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: i) X17S, X17T, or X17G; ii) X18S or X18R; iii) X56S, X56T, or X56D; iv) X94I; v) X106I, X106V, X106L, or X106W; vi) X173A, X173W, X173M, or X173K; vii) X194A, X194V, X194W, or X194T; viii) X196G; and ix) X198V, X198I, X198Y, or X198P relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 54. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: X94I, X96Y, X190A, X196G, X202A, X202G and/or X206W relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 54. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: X18G, X40H, X56V, X94A, X106E, X145E, X145, X173D, X194P, X198D, X202A, and X206Q relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 54.

In some embodiments, the engineered KRED polypeptides of the present disclosure have improved properties as compared to the engineered KRED polypeptide of SEQ ID NO: 152. In some embodiments, the engineered KRED polypeptides of the present disclosure are capable of selectively reducing tert-butyl rel-(3aR,6aS)-5-oxohexahydrocyclopenta[c]pyrrole-2(1H)-carboxylate (Substrate) to tert-butyl rel-(3aR,5s,6aS)-5-hydroxyhexahydrocyclopenta[c]pyrrole-2(1H)-carboxylate (Product) under suitable reaction conditions at a greater stereoselectivity (% diastereomeric excess) and/or activity (% conversion) than that of SEQ ID NO: 152.

In certain embodiments, the improved properties (e.g., reversed or increased diastereoselectivity (i.e., selectivity for (5s)-2 over (5r)-2) or activity (% conversion)) of the engineered KRED polypeptides of the present disclosure are based on mutating one or more of the following residues: X17, X18, X40, X56, X94, X96, X106, X145, X173, X190, X194, X196, X198, X202, and/or X206 relative to an amino acid sequence that is at least about is at least about 85%, 86%, 87%, 88%, 89%, 90%, 91%, 92%, 93%, 94%, 95%, 96%, 97%, 98%, or 99%, or is 100% identical to SEQ ID NO: 152. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on mutating one or more of the following residues: X94, X96, X190, X196, X202, and/or X206 relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 152. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on mutating one or more of the following residues: (i) X190 to a residue that is not tyrosine; (ii) X190 to a non-aromatic residue; (iii) X196 to an aliphatic residue or a small amino acid residue; (iv) X202 to a small amino acid residue; and/or (v) X206 to a residue that is not methionine, relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 152. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198I, X198Y, X198D, X198P, X202A, X202G, X206Q, X206N and/or X206W relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 152. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: i) X17S, X17T, or X17G; ii) X18S or X18R; iii) X56S, X56T, or X56D; iv) X94I; v) X106I, X106V, X106L, or X106W; vi) X173A, X173W, X173M, or X173K; vii) X194A, X194V, X194W, or X194T; viii) X196G; and ix) X198V, X198I, X198Y, or X198P relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 152. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: X94I, X96Y, X190A, X196G, X202A, X202G and/or X206W relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 152. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: X18G, X40H, X56V, X94A, X106E, X145E, X145, X173D, X194P, X198D, X202A, and X206Q relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 152.

In some embodiments, the engineered KRED polypeptides of the present disclosure have improved properties as compared to the engineered KRED polypeptide of SEQ ID NO: 256. In some embodiments, the engineered KRED polypeptides of the present disclosure are capable of selectively reducing tert-butyl rel-(3aR,6aS)-5-oxohexahydrocyclopenta[c]pyrrole-2(1H)-carboxylate (Substrate) to tert-butyl rel-(3aR,5s,6aS)-5-hydroxyhexahydrocyclopenta[c]pyrrole-2(1H)-carboxylate (Product) under suitable reaction conditions at a greater stereoselectivity (% diastereomeric excess) and/or activity (% conversion) than that of SEQ ID NO: 256.

In certain embodiments, the improved properties (e.g., reversed or increased diastereoselectivity (i.e., selectivity for (5s)-2 over (5r)-2) or activity (% conversion)) of the engineered KRED polypeptides of the present disclosure are based on mutating one or more of the following residues: X17, X18, X40, X56, X94, X96, X106, X145, X173, X190, X194, X196, X198, X202, and/or X206 relative to an amino acid sequence that is at least about 85%, 86%, 87%, 88%, 89%, 90%, 91%, 92%, 93%, 94%, 95%, 96%, 97%, 98%, or 99%, or is 100% identical to SEQ ID NO: 256. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on mutating one or more of the following residues: X94, X96, X190, X196, X202, and/or X206 relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 256. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on mutating one or more of the following residues: (i) X190 to a residue that is not tyrosine; (ii) X190 to a non-aromatic residue; (iii) X196 to an aliphatic residue or a small amino acid residue; (iv) X202 to a small amino acid residue; and/or (v) X206 to a residue that is not methionine, relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 256. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198I, X198Y, X198D, X198P, X202A, X202G, X206Q, X206N and/or X206W relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 256. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: i) X17S, X17T, or X17G; ii) X18S or X18R; iii) X56S, X56T, or X56D; iv) X94I; v) X106I, X106V, X106L, or X106W; vi) X173A, X173W, X173M, or X173K; vii) X194A, X194V, X194W, or X194T; viii) X196G; and ix) X198V, X198I, X198Y, or X198P relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 256. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: X94I, X96Y, X190A, X196G, X202A, X202G and/or X206W relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 256. In some embodiments, the improved properties (e.g., reversed or increased diastereoselectivity (i.e., selectivity for (5s)-2 over (5r)-2) or activity (% conversion)) of the engineered KRED polypeptides of the present disclosure are based on one or more of the following mutations: X18G, X40H, X56V, X94A, X106E, X145E, X145, X173D, X194P, X198D, X202A, and X206Q relative to an amino acid sequence that is at least about 85%, 86%, 87%, 88%, 89%, 90%, 91%, 92%, 93%, 94%, 95%, 96%, 97%, 98%, or 99%, or is 100% identical SEQ ID NO: 256.

Exemplary engineered KRED polypeptides of the present disclosure include, but are not limited to, engineered KRED polypeptides comprising an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99%, or is 100% identical to an amino acid sequence listed in Table 4, Table 5, Table 6, or Table 7. In some embodiments, the engineered KRED polypeptides of the present disclosure comprise an amino acid sequence listed in Table 4, Table 5, Table 6, or Table 7. In some embodiments, the engineered KRED polypeptides of the present disclosure comprise an amino acid sequence listed in Table 7.

In some embodiments, the engineered KRED polypeptides of the present disclosure comprise an amino acid sequence that is at least about 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% identical to SEQ ID NO: 392, 394, 396, 406, 408, 420, 422, 424, 426, 428, 430, 436, 438, 462, 464, 468, 470, 474, 476, 480, 482, and 490. In some embodiments, the engineered KRED polypeptides of the present disclosure comprise an amino acid sequence selected from the group consisting of: SEQ ID NO: 392, 394, 396, 406, 408, 420, 422, 424, 426, 428, 430, 436, 438, 462, 464, 468, 470, 474, 476, 480, 482, and 490.

In some embodiments, the engineered KRED polypeptides can have one or more additional amino acid residue differences as compared to a reference (e.g., parental) polypeptide (e.g., wild-type L. kefir ketoreductase (e.g., SEQ ID NO: 492) or engineered variant thereof (e.g., SEQ ID NO: 54, SEQ ID NO: 152, SEQ ID NO: 256). These differences can be amino acid insertions, deletions, substitutions, or any combination of such changes. In some embodiments, the amino acid sequence differences can comprise non-conservative, conservative, as well as a combination of non-conservative and conservative amino acid substitutions. In some embodiments, the amino acid difference can comprise the conservative substitutions as provided in Table 1. Various amino acid residue positions where such changes can be made are described herein.

In some embodiments, the engineered KRED polypeptides are derived from a naturally occurring KRED that includes one or more mutations corresponding to any of the mutations provided herein. One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally occurring KRED (e.g., having homology to L. kefir) that corresponds to any of the mutations described herein. For example, one of skill in the art would be able to generate mutations in L. brevis or L. minor that correspond to mutations in L. kefir (e.g., mutations described in Table 2, Table 4, Table 5, Table 6, and Table 7).

### Polynucleotides Encoding Engineered Ketoreductase Enzymes

The present disclosure provides polynucleotides encoding the engineered KRED enzymes disclosed herein. The polynucleotides may be operatively linked to a promotor or one or more heterologous regulatory sequences that control gene expression to create a recombinant polynucleotide capable of expressing the polypeptide. In some embodiments, the polypeptide utilizes codons optimized for specific desired expression systems. Expression constructs containing a heterologous polynucleotide encoding the engineered KRED polypeptides can be introduced into appropriate host cells to express the corresponding ketoreductase polypeptide.

Codons corresponding to the various amino acids are well known in the art. Thus, the availability of a polypeptide sequence provides one skilled in the art with a description of all the polynucleotides capable of encoding the subject polypeptide. The degeneracy of the genetic code, where the same amino acids are encoded by alternative or synonymous codons, allows an extremely large number of nucleic acids to be made, all of which encode the engineered KRED enzymes disclosed herein. Thus, having identified a particular amino acid sequence, those skilled in the art could make any number of different nucleic acids by simply modifying the sequence of one or more codons in a way which does not change the amino acid sequence of the protein. In this regard, the present disclosure specifically contemplates each and every possible variation of polynucleotides that could be made by selecting combinations based on the possible codon choices, and all such variations are to be considered specifically disclosed for any polypeptide disclosed herein, including the amino acid sequences presented herein (e.g., Table 4, Table 5, Table 6, or Table 7).

In one embodiment, the polynucleotides of the present disclosure encode any of the engineered KRED polypeptides described herein comprising an amino acid sequence with one or more amino acid differences as compared to a reference (e.g., parental) amino acid sequence (e.g., wild-type KRED (e.g., L. kefir ketoreductase) or an engineered KRED amino acid sequence (e.g., SEQ ID NO: 54)). In some embodiments, the polynucleotides of the present disclosure encode any of the engineered KRED polypeptides described herein that are derived from a wild-type ketoreductase (e.g., L. kefir, L. brevis, L. minor). In some embodiments, the polynucleotides of the present disclosure encode any of the engineered KRED polypeptides described herein comprising an amino acid sequence with one or more amino acid differences as compared to a wild-type L. kefir KRED polypeptide.

In some embodiments, the polynucleotides of the present disclosure encode any of the engineered KRED polypeptides described herein comprising an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 54, 152, 256, or 492; and comprising one or more amino acid differences relative to said amino acid sequence selected from: i) X17S, X17T, or X17G; ii) X18S or X18R; iii) X56S, X56T, or X56D; iv) X94I; v) X106I, X106V, X106L, or X106W; vi) X173A, X173W, X173M, or X173K; vii) X194A, X194V, X194W, or X194T; viii) X196G; and ix) X198V, X198I, X198Y, or X198P.

In certain embodiments, the polynucleotides of the present disclosure encode an amino acid sequence at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 54, 152, 256, or 492 comprising a substitution, deletion, addition or insertion of one or more amino acid residues selected from Table 2, Table 4, Table 5, Table 6, or Table 7 relative to said amino acid sequence. In some embodiments, the polynucleotides of the present disclosure encode an amino acid sequence with one or more amino acid differences selected from: X17, X18, X40, X56, X94, X96, X106, X145, X173, X190, X194, X196, X198, X202, and/or X206 relative to an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 54, SEQ ID NO: 152, SEQ ID NO: 256, or SEQ ID NO: 492. In some embodiments, the polynucleotides of the present disclosure encode an amino acid sequence with one or more amino acid differences selected from: X94, X96, X190, X196, X202, and/or X206 relative to an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 54, SEQ ID NO: 152, SEQ ID NO: 256, or SEQ ID NO: 492. In some embodiments, the polynucleotides of the present disclosure encode an amino acid sequence with one or more amino acid differences selected from: (i) X190 to a residue that is not tyrosine; (ii) X190 to a non-aromatic residue; (iii) X196 to an aliphatic residue or a small amino acid residue; (iv) X202 to a small amino acid residue; and/or (v) X206 to a residue that is not methionine, relative to an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 54, SEQ ID NO: 152, SEQ ID NO: 256, or SEQ ID NO: 492.

In some embodiments, the polynucleotides of the present disclosure encode an amino acid sequence with one or more amino acid differences selected from: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198I, X198Y, X198D, X198P, X202A, X202G, X206Q, X206N and/or X206W relative to an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 54, SEQ ID NO: 152, SEQ ID NO: 256, or SEQ ID NO: 492. In some embodiments, the polynucleotides of the present disclosure encode an amino acid sequence with one or more amino acid differences selected from: X94I, X96Y, X190A, X196G, X202A, X202G and/or X206W relative to an amino acid.

In some embodiments, the polynucleotides of the present disclosure encode an amino acid sequence selected from Table 4, Table 5, Table 6 or Table 7. In some embodiments, the polynucleotides of the present disclosure encode an amino acid sequence listed in Table 7. In some embodiments, the polynucleotides of the present disclosure encode any of the engineered KRED polypeptides described herein comprising an amino acid sequence that is at least about 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% identical to an amino acid sequence selected from the group consisting of SEQ ID NOs: 392, 394, 396, 406, 408, 420, 422, 424, 426, 428, 430, 436, 438, 462, 464, 468, 470, 474, 476, 480, 482, and 490. In some embodiments, the polynucleotides of the present disclosure encode any of the engineered KRED polypeptides described herein comprising an amino acid sequence selected from the group consisting of SEQ ID NOs: 392, 394, 396, 406, 408, 420, 422, 424, 426, 428, 430, 436, 438, 462, 464, 468, 470, 474, 476, 480, 482, and 490.

In some embodiments, the KRED polynucleotides of the present disclosure comprise a nucleic acid sequence listed in Table 4, Table 5, Table 6, or Table 7. In some embodiments, the KRED polynucleotides of the present disclosure comprise a nucleic acid sequence listed in Table 7. In some embodiments, the KRED polynucleotides of the present disclosure comprise a nucleic acid sequence that is at least about 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% identical to SEQ ID NO: 391, 393, 395, 405, 407, 419, 421, 423, 425, 427, 429, 435, 437, 461, 463, 467, 469, 473, 475, 479, 481, or 489. In some embodiments, the KRED polynucleotides of the present disclosure comprise a nucleic acid sequence selected from SEQ ID NO: 391, 393, 395, 405, 407, 419, 421, 423, 425, 427, 429, 435, 437, 461, 463, 467, 469, 473, 475, 479, 481, or 489.

In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. By way of example, a polynucleotide can be codon optimized for expression in Escherichia coli (“E. coli”), but otherwise encode a naturally occurring KRED of L. kefir.

In certain embodiments, all codons need not be replaced to optimize the codon usage of the KREDs since the natural sequence will comprise preferred codons and because use of preferred codons may not be required for all amino acid residues. Consequently, codon optimized polynucleotides encoding the KRED enzymes may contain preferred codons at about 40%, 50%, 60%, 70%, 80%, or greater than 90% of codon positions of the full length coding region.

In various embodiments, an isolated polynucleotide encoding an engineered KRED polypeptide of the present disclosure may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3rd Ed., Cold Spring Harbor Laboratory Press; and Current Protocols in Molecular Biology, Ausubel. F. ed., Greene Pub. Associates, 1998, updates to 2006.

For bacterial host cells, suitable promoters for directing transcription of the nucleic acid constructs of the present disclosure, include the promoters obtained from the E. coli lac operon, Streptomyces coelicolor agarase gene (dagA), Bacillus subtilis levansucrase gene (sacB), Bacillus lichenformis alpha-amylase gene (amyL), Bacillus stearothermophilus maltogenic amylase gene (amyM), Bacillus amyloliquefaciens alpha-amylase gene (amyQ), Bacillus lichenformis penicillinase gene (penP), Bacillus subtilis xylA and xylB genes, and prokaryotic beta-lactamase gene (Villa-Kamaroff et al., 1978, Proc. Natl Acad. Sci. USA 75: 3727-3731), as well as the tac promoter (DeBoer et al., 1983, Proc. Natl Acad. Sci. USA 80: 21-25).

For filamentous fungal host cells, suitable promoters for directing the transcription of the nucleic acid constructs of the present disclosure include promoters obtained from the genes for Aspergillus oryzae TAKA amylase, Rhizomucor miehei aspartic proteinase, Aspergillus niger neutralalpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

In a yeast host, useful promoters can be from the genes for Saccharomyces cerevisiae enolase (ENO-1), Saccharomyces cerevisiae galactokinase (GALI), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., 1992, Yeast 8:423-488.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention. For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYCl), and Saccharomyces cerevisiae glyceraldehyde-3-phosphate dehydrogenase. Other useful terminators for yeast host cells are described by Romanos et al., 1992, supra.

The control sequence may also be a suitable leader sequence, a non-translated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence, which, when transcribed, is recognized by the host cell as a signal to add polyadenosine residues to transcribed mRNA. Any polyadenylation sequence that is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are described by Guo and Sherman, 1995, Mol Cell Bio 15:5983-5990.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. The foreign signal peptide coding region may be required where the coding sequence does not naturally contain a signal peptide coding region.

Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for bacterial host cells are the signal peptide coding regions obtained from the genes for Bacillus NCIB 11837 maltogenic amylase, Bacillus stearothermophilus alpha-amylase, Bacillus lichenformis subtilisin, Bacillus lichenformis beta-lactamase, Bacillus stearothermophilus neutral proteases (nprT, nprS, nprM), and Bacillus subtilis prsA. Further signal peptides are described by Simonen and Palva, 1993, Microbiol Rev 57: 109-137.

Effective signal peptide coding regions for filamentous fungal host cells can be the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

Useful signal peptides for yeast host cells can be from the genes for Saccharomyces cerevisiae alpha-factor and Saccharomyces cerevisiae invertase. Other useful signal peptide coding regions are described by Romanos et al., 1992, supra.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences, which allow the regulation of the expression of the polypeptide relative to the growth of the host cell. Examples of regulatory sequences are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GALI system. In filamentous fungi, suitable regulatory sequences include the TAKA alpha-amylase promoter, Aspergillus niger glucoamylase promoter, and Aspergillus oryzae glucoamylase promoter.

Other examples of regulatory sequences are those that allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding the KRED polypeptide of the present invention would be operably linked with the regulatory sequence.

Thus, in another embodiment, the present disclosure is also directed to a recombinant expression vector comprising a polynucleotide encoding an engineered KRED polypeptide or a variant thereof, and one or more expression regulating regions such as a promoter and a terminator, a replication origin, etc., depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the polypeptide at such sites.

Alternatively, the nucleic acid sequences of the present disclosure may be expressed by inserting a nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The expression vector of the present invention preferably contains one or more selectable markers, which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus lichenformis, or markers, which confer antibiotic resistance such as ampicillin, kanamycin, chloramphenicol (see Example 1) or tetracycline resistance. Suitable markers for yeast host cells are ADE2, HIS3, LEU2, LYS2, MET3, TRP1, and URA3.

Selectable markers for use in a filamentous fungal host cell include, but are not limited to, amdS (acetamidase), argB (omithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus.

The expression vectors of the present invention preferably contain an element(s) that permits integration of the vector into the host cell's genome or autonomous replication of the vector in the cell independent of the genome. For integration into the host cell genome, the vector may rely on the nucleic acid sequence encoding the polypeptide or any other element of the vector for integration of the vector into the genome by homologous or nonhomologous recombination.

Alternatively, the expression vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are PISA ori or the origins of replication of plasmids pBR322, pUC19, pACYC177 (which plasmid has the PISA ori), or pACYC184 permitting replication in E. coli, and pUB110, pEI94, pTAI 060, or pAMP1 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes it's functioning temperature-sensitive in the host cell (see, e.g., Ehrlich, 1978, Proc Natl Acad Sci. USA 75:1433).

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Many of the expression vectors for use in the present invention are commercially available. Suitable commercial expression vectors include p3xFLAG™™ expression vectors from Sigma-Aldrich Chemicals, St. Louis MO., which includes a CMV promoter and hGH polyadenylation site for expression in mammalian host cells and a pBR322 origin of replication and ampicillin resistance markers for amplification in E. coli. Other suitable expression vectors are pBluescriptII SK(−) and pBK-CMV, which are commercially available from Stratagene, LaJolla CA, and plasmids derived from pBR322 (Gibco BRL), pUC (Gibco BRL), pREP4, pCEP4 (Invitrogen) or pPoly (Lathe et al., 1987, Gene 57:193-201).

### Host Cells and Expression Vectors for Expression of Ketoreductase Polypeptides

The present disclosure further provides host cells comprising any of the polynucleotides and/or expression vectors described herein. The host cells can be used for the expression and isolation of the engineered KRED enzymes described herein, or, alternatively, they can be used directly for the conversion of the substrate (Compound 1) to the product ((5s)-2).

In some embodiments, the host cell comprises a polynucleotide encoding an engineered KRED polypeptide operatively linked to one or more control sequences for expression of the KRED enzyme in the host cell. Host cells for use in expressing the KRED polypeptides encoded by the expression vectors of the present invention are well known in the art and include but are not limited to, bacterial cells, such as E. coli, L. kefir, L. brevis, L. minor, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Appropriate culture mediums and growth conditions for the above-described host cells are well known in the art.

Polynucleotides for expression of KREDs may be introduced into cells by various methods known in the art. Nonlimiting techniques include electroporation, biolistic particle bombardment, liposome mediated transfection, calcium chloride transfection, and protoplast fusion. Various methods for introducing polynucleotides into cells will be apparent to the skilled artisan.

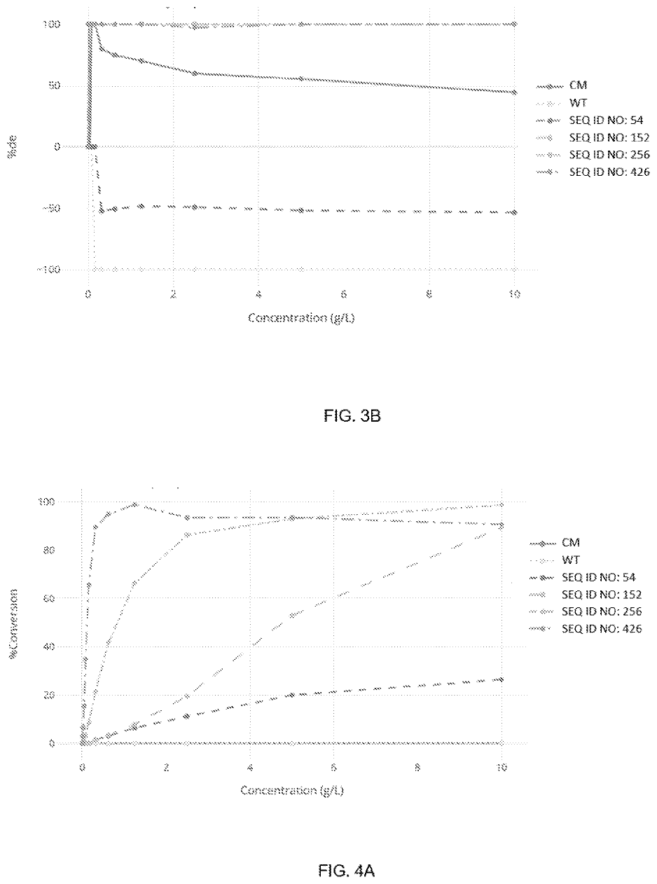

An exemplary host cell is E. coli W3110. The expression vector was created by operatively linking a polynucleotide encoding an engineered KRED polypeptide into the plasmid pCK110900 (vector depicted as FIG. 3 in U.S. Patent App. Pub. 20060195947, which is hereby incorporated by reference herein) operatively linked to the lac promoter under control of the lacI repressor. The expression vector also contained the P15a origin of replication and the chloramphenicol (CAM) resistance gene. Cells containing the subject polynucleotide in E. coli W3110 were isolated by subjecting the cells to chloramphenicol selection.

### Methods of Generating Engineered Ketoreductase Polypeptides

The present disclosure provides methods for generating or producing any of the engineered KRED polynucleotides and polypeptides as disclosed herein. In general, the methods for generating or producing an engineered KRED polynucleotide or polypeptide as provided herein include introducing one or more differences (e.g., a substitution, deletion, addition or insertion) into the nucleic acid or amino acid sequence of a KRED polypeptide.

In some embodiments, to make the engineered KRED polynucleotides and polypeptides of the present disclosure, a naturally occurring KRED enzyme that catalyzes the reduction reaction is obtained (or derived) (e.g., from L. kefir, L. minor or L. brevis) for use as a parental polynucleotide sequence. In some embodiments, the parental polynucleotide sequence is obtained (or derived) from a naturally occurring ketoreductase enzyme selected from L. kefir, L. minor or L. brevis. In some embodiments, the parental polynucleotide sequence is obtained (or derived) from L. kefir (e.g., SEQ ID NO: 492). In some embodiments, to make the engineered KRED polynucleotides and polypeptides of the present disclosure, an engineered variant of a naturally occurring KRED enzyme that catalyzes the reduction reaction is obtained (or derived) (e.g., SEQ ID NO: 54, 152, or 256) for use as a parental polynucleotide sequence. In some embodiments, the parental polynucleotide sequence is obtained (or derived) from an engineered KRED variant selected from Table 4, Table 5, or Table 6. In some embodiments, the parental polynucleotide sequence is obtained (or derived) from an engineered KRED variant selected from SEQ ID NO: 54, SEQ ID NO: 152, or SEQ ID NO: 256.

In some embodiments, the parental polynucleotide sequence is codon optimized to enhance expression of the KRED polypeptide in a specified host cell. As an illustration, the parental polynucleotide sequence encoding the wild-type KRED polypeptide of L. kefir was constructed from oligonucleotides prepared based upon the known polypeptide sequence of L. kefir KRED sequence (available at UniProKB Accession No. Q6WVP7). The parental polynucleotide sequence, designated as SEQ ID NO: 492, was codon optimized for expression in E. coli and the codon-optimized polynucleotide cloned into an expression vector, placing the expression of the ketoreductase gene under the control of the lac promoter and lacI repressor gene. Clones expressing the active ketoreductase in E. coli were identified and the genes sequenced to confirm their identity. The parental sequence was then utilized as the starting point for most experiments and library construction of engineered KREDs evolved from the L. kefir ketoreductase (e.g., SEQ ID NO: 54, SEQ ID NO: 152, or SEQ ID NO: 256).

The engineered KRED polypeptides herein can be obtained by subjecting the parental polynucleotide encoding the naturally occurring ketoreductase (e.g., L. kefir) or a variant thereof (e.g., SEQ ID NO: 54, 152, 256) to mutagenesis and/or directed evolution methods. In general, the engineered KRED polypeptides generated or produced as provided herein will have an amino acid sequence with one or more amino acid differences as compared to the parental KRED (e.g., wild-type KRED (e.g., L. kefir) or engineered KRED variant (e.g., SEQ ID NO: 54, 152, or 256)).

In some embodiments, an engineered KRED polypeptide of the present disclosure can be obtained by subjecting a parental polynucleotide encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 54, 152, 256, or 492 to mutagenesis to introduce a substitution, deletion, addition or insertion of one or more amino acid residues selected from Table 2, Table 4, Table 5, Table 6, or Table 7 relative to said amino acid sequence. In some embodiments, an engineered KRED polypeptide of the present disclosure can be obtained by subjecting a parental polynucleotide encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 54, 152, 256, or 492 to mutagenesis to introduce a substitution, deletion, addition or insertion of one or more amino acid residues selected from Table 2 and one or more amino acid residues selected from Table 4, Table 5, Table 6, or Table 7 relative to said amino acid sequence.

In some embodiments, an engineered KRED polypeptide of the present disclosure can be obtained by introducing one or more amino acid differences selected from: X17, X18, X40, X56, X94, X96, X106, X145, X173, X190, X194, X196, X198, X202, and/or X206 relative to an amino acid sequence that is at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 54, SEQ ID NO: 152, SEQ ID NO: 256, or SEQ ID NO: 492. In some embodiments, an engineered KRED polypeptide of the present disclosure can be obtained by introducing one or more amino acid differences selected from: X94, X96, X190, X196, X202, and/or X206 relative to an amino acid sequence that is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 54, SEQ ID NO: 152, SEQ ID NO: 256, or SEQ ID NO: 492. In some embodiments, an engineered KRED polypeptide of the present disclosure can be obtained by introducing one or more amino acid differences selected from: (i) X190 to a residue that is not tyrosine; (ii) X190 to a non-aromatic residue; (iii) X196 to an aliphatic residue or a small amino acid residue; (iv) X202 to a small amino acid residue; and/or (v) X206 to a residue that is not methionine, relative to an amino acid sequence that is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 54, SEQ ID NO: 152, SEQ ID NO: 256, or SEQ ID NO: 492.

In some embodiments, an engineered KRED polypeptide of the present disclosure can be obtained by introducing one or more amino acid differences selected from: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198I, X198Y, X198D, X198P, X202A, X202G, X206Q, X206N and/or X206W relative to an amino acid sequence that is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 54, SEQ ID NO: 152, SEQ ID NO: 256, or SEQ ID NO: 492. In some embodiments, an engineered KRED polypeptide of the present disclosure can be obtained by subjecting a parental polynucleotide encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 54, 152, 256, or 492 to mutagenesis to introduce one or more amino acid differences selected from: i) X17S, X17T, or X17G; ii) X18S or X18R; iii) X56S, X56T, or X56D; iv) X94I; v) X106I, X106V, X106L, or X106W; vi) X173A, X173W, X173M, or X173K; vii) X194A, X194V, X194W, or X194T; viii) X196G; and ix) X198V, X198I, X198Y, or X198P, relative to said amino acid sequence. In some embodiments, an engineered KRED polypeptide of the present disclosure can be obtained by introducing one or more amino acid differences selected from: X94I, X96Y, X190A, X196G, X202A, X202G and/or X206W relative to an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 54, SEQ ID NO: 152, SEQ ID NO: 256, or SEQ ID NO: 492. In some embodiments, an engineered KRED polypeptide of the present disclosure can be obtained by introducing one or more amino acid differences selected from: X18G, X40H, X56V, X94A, X106E, X145E, X145, X173D, X194P, X198D, X202A, and X206Q relative to an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 54, SEQ ID NO: 152, SEQ ID NO: 256, or SEQ ID NO: 492.

In some embodiments, the engineered KRED polypeptides generated or produced by any of the methods provided herein have an amino acid sequence that is at least about 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% identical to an amino acid sequence selected from the group consisting of SEQ ID NOs: 392, 394, 396, 406, 408, 420, 422, 424, 426, 428, 430, 436, 438, 462, 464, 468, 470, 474, 476, 480, 482, and 490. In some embodiments, the engineered KRED polypeptides generated or produced by any of the methods provided herein have an amino acid sequence selected from the group consisting of: SEQ ID NO: 392, 394, 396, 406, 408, 420, 422, 424, 426, 428, 430, 436, 438, 462, 464, 468, 470, 474, 476, 480, 482, and 490.

In some embodiments, the engineered KRED polypeptides generated or produced by any of the methods provided herein have an amino acid sequence selected from Table 4 (SEQ ID NOs: 4-52), Table 5 (SEQ ID NOs: 56-186), Table 6 (SEQ ID NOs: 188-390) or Table 7 (SEQ ID NOs: 392-490). In some embodiments, the engineered KRED polypeptides produced by any of the methods provided herein have an amino acid sequence selected from Table 7 (SEQ ID NOs: 392-490).

As discussed herein, mutagenesis and/or directed evolution methods are well known in the art. An exemplary directed evolution technique is mutagenesis and/or DNA shuffling as described in Stemmer, 1994, Proc Natl Acad Sci USA 91:10747-10751; WO 95/22625; WO 97/0078; WO 97/35966; WO 98/27230; WO 00/42651; WO 01/75767 and U.S. Pat. No. 6,537,746. Other directed evolution procedures that can be used include, among others, staggered extension process (StEP), in vitro recombination (Zhao et al., 1998, Nat. Biotechnol. 16:258-261), mutagenic PCR (Caldwell et al., 1994, PCR Methods Appl. 3:S136-S140), and cassette mutagenesis (Black et al., 1996, Proc Natl Acad Sci USA 93:3525-3529).

The clones obtained following mutagenesis treatment are screened for engineered KREDs having a desired improved enzyme property (e.g., reversed diastereoselectivity, increased selectivity (i.e., (5s)-2 over (5r)-2), increased activity (% conversion), etc.). Measuring enzyme activity from the expression libraries can be performed using the standard biochemistry technique of monitoring the rate of decrease (via a decrease in absorbance or fluorescence) of NADH or NADPH concentration, as it is converted into NAD⁺ or NADP⁺. In this reaction, the NADH or NADPH is consumed (oxidized) by the ketoreductase as the ketoreductase reduces a ketone substrate to the corresponding hydroxyl group. The rate of decrease of NADH or NADPH concentration, as measured by the decrease in absorbance or fluorescence, per unit time indicates the relative (enzymatic) activity of the KRED polypeptide in a fixed amount of the lysate (or a lyophilized powder made therefrom). Where the improved enzyme property desired is thermal stability, enzyme activity may be measured after subjecting the enzyme preparations to a defined temperature and measuring the amount of enzyme activity remaining after heat treatments. Clones containing a polynucleotide encoding a KRED are then isolated, sequenced to identify the nucleotide sequence changes (if any), and used to express the enzyme in a host cell.

In some embodiments, multiple rounds of mutagenesis treatments may be used for screening engineered KREDs having a desired improved enzyme property (e.g., reversed or increased diastereoselectivity (i.e., selectivity for (5s)-2 over (5r)-2) or activity (% conversion)). For example, KRED polypeptide variants derived from L. kefir (e.g., SEQ ID NO: 54) were selected with suitable initial activity/selectivity toward the formation of compound (5s)-2, and those variants were used as the “backbone” for an additional rounds of codon optimization and directed evolution. Further rounds of directed evolution may then be carried out using a gene encoding the most improved polypeptide from each round (e.g., SEQ ID NO: 152; SEQ ID NO: 256) as the parent backbone sequence for the subsequent round of evolution to identify exemplary improved engineered KRED polypeptide sequences.

Where the sequence of the engineered polypeptide is known, the polynucleotides encoding the KRED enzyme can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides and oligonucleotides of the invention can be prepared by chemical synthesis using, e.g., the classical phosphoramidite method described by Beaucage et al., 1981, Tet Lett 22:1859-69, or the method described by Matthes et al., 1984, EMBO J. 3:801-05, e.g., as it is typically practiced in automated synthetic methods. According to the phosphoramidite method, oligonucleotides are synthesized, e.g., in an automatic DNA synthesizer, purified, annealed, ligated and cloned in appropriate vectors. In addition, essentially any nucleic acid can be obtained from any of a variety of commercial sources, such as The Midland Certified Reagent Company, Midland, TX, The Great American Gene Company, Ramona, CA, ExpressGen Inc. Chicago, IL, Operon Technologies Inc., Alameda, CA, and many others.

Engineered KRED enzymes expressed in a host cell can be recovered from the cells and or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic B™ from Sigma-Aldrich of St. Louis MO.

Chromatographic techniques for isolation of the KRED polypeptide include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques may be used to isolate the engineered KRED enzymes. For affinity chromatography purification, any antibody which specifically binds the KRED polypeptide may be used. For the production of antibodies, various host animals (e.g., rabbits, mice, rats, etc.) may be immunized by injection with a compound. The compound may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (Bacillus Calmette Guerin) and Corynebacterium parvum.

In some embodiments, the methods provided herein are capable of generating or producing engineered KREDs that have reversed stereoselectivity (e.g., diastereoselectivity (i.e., selectivity for (5s)-2 over (5r)-2)). In particular, the methods provided herein reverse the diastereoselectivity of a KRED polypeptide. For example, wild-type L. kefir KRED (e.g., SEQ ID NO: 492) displays specificity toward the formation of the (trans) alcohol product (i.e., (5r)-2) when reducing substrate (i.e., Compound 1). However, upon introducing the one or more mutations as provided according to the methods of the present disclosure, the resulting engineered KRED polypeptide produced exhibits a reversed selectivity and displays a strong specificity towards the formation of the (cis) alcohol product (i.e., (5s)-2) diastereomer).

In some embodiments, the parental KRED polynucleotide or polypeptide used in the methods for reversing diastereoselectivity is a wild-type KRED (e.g., L. kefir) that exhibits specificity towards the formation of the (trans) alcohol product (i.e., (5r)-2). In some embodiments, the wild-type KRED is L. kefir, L. brevis, or L. minor. In some embodiments, the wild-type KRED polypeptide is L. kefir (e.g., SEQ ID NO: 492). Additional parental KRED polynucleotides or polypeptides used in the methods for reversing diastereoselectivity include engineered variants of a wild-type a KRED that exhibit specificity towards the formation of the (trans) alcohol product (i.e., (5r)-2).

In some embodiments, the methods provided herein are also capable of generating or producing an engineered ketoreductase polypeptide that has an increased stereoselectivity (e.g., diastereoselectivity (i.e., selectivity for (5s)-2 over (5r)-2)). In particular, the methods provided herein increase the stereoselectively or diastereoselectivity of a KRED polypeptide towards the formation of the (cis) alcohol product (i.e., (5s)-2). When reducing the substrate (i.e., Compound 1), a KRED polypeptide (e.g., an engineered variant of L. kefir (e.g., SEQ ID NO: 54)) may display a low specificity towards the formation of the (cis) alcohol product (i.e., (5s)-2). However, upon introducing the one or more mutations as provided according to the methods of the present disclosure, the engineered KRED polypeptide exhibits an increased selectivity and strong specificity towards the formation of the (cis) alcohol product (i.e., (5s)-2 diastereomer).

In some embodiments, the parental KRED polynucleotide or polypeptide used in the methods for increasing stereoselectively is an engineered variant of a wild-type L. kefir KRED polypeptide (e.g., SEQ ID NO: 54, 152, 256). In some embodiments, the engineered variant for use in the methods for increasing stereoselectively is selected from an amino acid sequence in Table 4, Table 5, or Table 6. In some embodiments, the engineered variant for use in the methods for increasing stereoselectively is selected from an amino acid sequence of SEQ ID NO: 54, SEQ ID NO: 152, or SEQ ID NO: 256. In some embodiments, the methods provided herein increase the level of stereoselectively (e.g., % diastereomeric excess) in a KRED polypeptide towards the formation of the (cis) alcohol product (i.e., (5s)-2) to at least about 85%, 86%, 87%, 88%, 89%, 90%, 91%, 92%, 93%, 94%, 95%, 96%, 97%, 98%, 99%, or 100%. In some embodiments, the methods provided herein increase the level of stereoselectively (e.g., % diastereomeric excess) in a KRED polypeptide towards the formation of the (cis) alcohol product (i.e., (5s)-2) to at least about 96%. In some embodiments, the methods provided herein increase the level of stereoselectively (e.g., % diastereomeric excess) in a KRED polypeptide towards the formation of the (cis) alcohol product (i.e., (5s)-2) to at least about 99%.

In some embodiments, the methods provided herein are also capable of generating or producing an engineered ketoreductase polypeptide with increased activity (e.g., % conversion). In particular, the methods provided herein increase the activity of a KRED polypeptide to convert substrate (e.g., Compound 1) to product (e.g., (5s)-2). When reducing the substrate (e.g., Compound 1) to product (e.g., (5s)-2), a KRED polypeptide (e.g., wild-type or an engineered variant (e.g., SEQ ID NO: 54)) may display a low conversion rate. However, upon introducing the one or more mutations as provided according to the methods of the present disclosure, the engineered KRED polypeptide exhibits an increase in the conversion rate of substrate (e.g., Compound 1) to product (e.g., (5s)-2).

In some embodiments, the parental KRED polynucleotide or polypeptide used in the methods for increasing activity (e.g., % conversion) is a wild-type KRED (e.g., L. kefir). In some embodiments, the wild-type KRED is L. kefir, L. brevis, or L. minor. In some embodiments, the wild-type KRED is L. kefir (e.g., SEQ ID NO: 492). Additional parental KRED polynucleotides or polypeptides used in the methods for increasing activity include engineered variants of a wild-type a KRED (e.g., SEQ ID NO: 54, 152, or 256). In some embodiments, the engineered variant is selected from Table 4, Table 5, or Table 6. In some embodiments, the engineered variant is selected from SEQ ID NO: 54, SEQ ID NO: 152, or SEQ ID NO: 256. In some embodiments, the methods provided herein increase the activity (e.g., % conversion) of an engineered KRED to a conversion % of at least about 85%, at least about 90%, at least about 95%, at least about 96%, at least about 97%, at least about 98%, at least about 99.0%, or 100%. In some embodiments, the methods provided herein increase the activity (e.g., conversion rate or desired product) of an engineered KRED relative to the parental KRED with a fold improvement over positive control (FIOP) greater than about 1.75, greater than about 2.00, greater than about 2.25, greater than about 2.50, greater than about 2.75, greater than about 3.00, greater than about 3.25, greater than about 3.50, greater than about 3.75, greater than about 4.00, greater than about 4.25, greater than about 4.50, greater than about 4.75, greater than about 5.00, greater than about 5.25, greater than about 5.50, greater than about 5.75, greater than about 6.00, greater than about 6.25, or greater than about 6.50. In some embodiments, the methods provided herein increase the activity (e.g., conversion rate or desired product) of an engineered KRED relative to the parental KRED with a FIOP greater than about 2.25, preferably greater than 3.00. In some embodiments, the engineered polypeptide is capable of reducing Substrate to Product with a FIOP conversion rate greater than about 2.25 and a diastereoselectivity greater than about 99% as compared to the parental KRED.

Any of the engineered KRED polypeptides used in any of the methods provided herein may be derived from a different naturally occurring KRED by introducing one or more mutations corresponding to any of the mutations provided herein. One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally occurring KRED (e.g., having homology to L. kefir) that corresponds to any of the mutations described herein. For example, one of skill in the art would be able to generate mutations in L. brevis or L. minor that correspond to mutations in L. kefir (e.g., mutations described in Table 2, Table 4, Table 5, Table 6, or Table 7).

Any of the engineered KRED polypeptides used in the methods herein can have one or more additional modifications. The modifications can include substitutions, deletions, and insertions. The substitutions can be non-conservative substitutions, conservative substitutions, or a combination of non-conservative and conservative substitutions.

### Methods of Using the Engineered Ketoreductase Polypeptides and Compounds Prepared Therewith

The present disclosure provides methods for using any of the engineered KRED polynucleotides, polypeptides or compositions thereof as provided herein. In some embodiments, the engineered KREDs of the present disclosure can be used in the form of whole cell, crude extract, isolated enzyme, or purified enzyme. In some embodiments, the engineered KREDs of the present disclosure can be used alone or in an immobilized form (e.g., immobilization on a resin).

In the methods of the present disclosure, an engineered KRED polypeptide of the present disclosure is used to selectively catalyze the reduction of a bicyclic ketone substrate. In some embodiments, the method includes the step of contacting or incubating the bicyclic ketone with a KRED polypeptide as disclosed herein under reaction conditions suitable for reducing or converting the substrate to the product compound.

In the methods of the present disclosure, the bicyclic ketone substrate is of formula (IA):

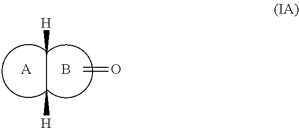

wherein:
the A ring and the B ring together represent a fused cycloalkyl ring, e.g., C₆-C₁₂ cycloalkyl or fused heterocyclyl ring, e.g., 6-12 membered heterocyclyl,
wherein the fused cycloalkyl or heterocyclyl is optionally substituted with at least one occurrence of R¹, e.g., one to four R¹,
each R¹ is independently selected from an amine protecting group, e.g., tert-butyloxycarbonyl (Boc), N-carboxybenzyl (Cbz), C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, 5-20 membered heteroaryl, hydroxyl, halogen, e.g., F, C₁-C₂₀haloalkyl, e.g., —CF₃, —OR^{c}, —NR^{c}R^{c}, —(CH₂)ₙCOOR^{c}, —(CH₂)ₙ—C(═O)R^{c}, and —(CH₂)ₙ—C(═O)NR^{c}R^{c};
wherein the alkyl, alkenyl, and alkynyl are each optionally substituted by one or more R^{a}, e.g., one to six R^{a},
wherein the cycloalkyl, aryl, arylalkyl, heterocyclyl, and heteroaryl are each optionally substituted by one or more R^{b}, e.g., one to six R^{b};
each R^{a} is at each occurrence independently selected from C₃-C₁₀cycloalkyl, C₆-C₁₄aryl, C₇-C₂₀arylalkyl, 3-14 membered heterocyclyl, and 5-20 membered heteroaryl, halogen, e.g., F, haloalkyl, e.g., C₁-C₂₀haloalkyl, e.g., —CF₃, —OR^{c}, —NR^{c}R^{c}, —(CH₂)ₙCOOR^{c}, —(CH₂)ₙ—C(═O)R^{c}, and —(CH₂)ₙ—C(═O)NR^{c}R^{c};
each R^{b} is at each occurrence independently selected from halogen, C₁-C₂₀haloalkyl, e.g., —CF₃, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c}, C₁-C₂₀alkyl, C₂-C₂₀alkenyl, and C₂-C₂₀alkynyl;
each R^{c} is at each occurrence independently selected from H, C₁-C₂₀alkyl, C₂-C₂₀alkenyl, and C₂-C₂₀alkynyl, each optionally substituted by one or more R^{b}, e.g., one to six R^{b}; and
n is 0, 1, 2, 3, 4, 5 or 6, e.g., 0, 1, 2 or 3.

In an embodiment, the A ring and the B ring together represent a fused 6-12 membered heterocyclyl comprising at least one nitrogen heteroatom. In a further embodiment, the nitrogen heteroatom of the bicyclic ketone is bonded to an amine protecting group, e.g., tert-butyloxycarbonyl (Boc), N-carboxybenzyl (Cbz).

In an embodiment, the bicyclic ketone substrate is of formula (IA)-I

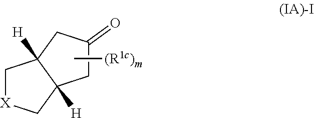

wherein:
X is selected from N—R^{1a}, CH₂ and CH—R^{1b};
R^{1a} is selected from an amine protecting group, e.g., tert-butyloxycarbonyl (Boc), N-carboxybenzyl (Cbz), C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, and 5-20 membered heteroaryl;
R^{1b} is selected from C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, and 5-20 membered heteroaryl;
wherein the alkyl, alkenyl, and alkynyl of R^{1a} or R^{1b} are each optionally substituted by one to six R^{a};
wherein the cycloalkyl, aryl, arylalkyl, heterocyclyl, and heteroaryl of Ria or R^{1b} are each optionally substituted by one to six R^{b};
each R^{1c} is at each occurrence independently selected from C₁-C₂₀alkyl, C₂-C₂₀alkenyl, C₂-C₂₀alkynyl, C₅-C₁₀cycloalkyl, C₆-C₁₄aryl, C₇-C₂₀arylalkyl, 3-14 membered heterocyclyl, and 5-20 membered heteroaryl, hydroxyl, halogen, e.g., F, C₁-C₂₀haloalkyl, e.g., —CF₃, —OR^{c}, —NR^{c}R^{c}, —(CH₂)ₙCOOR^{c}, —(CH₂)ₙ—C(═O)R^{c}, and —(CH₂)ₙ—C(═O)NR^{c}R^{c};
each R^{a} is at each occurrence independently selected from C₃-C₁₀cycloalkyl, C₆-C₁₄aryl, C₇-C₂₀arylalkyl, 3-14 membered heterocyclyl, and 5-20 membered heteroaryl, halogen, e.g., F, haloalkyl, e.g., C₁-C₂₀haloalkyl, e.g., —CF₃, —OR^{c}, —NR^{c}R^{c}, —(CH₂)ₙCOOR^{c}, —(CH₂)ₙ—C(═O)R^{c}, and —(CH₂)ₙ—C(═O)NR^{c}R^{c};
each R^{b} is at each occurrence independently selected from halogen, C₁-C₂₀haloalkyl, e.g., —CF₃, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c}, C₁-C₂₀alkyl, C₂-C₂₀alkenyl, and C₂-C₂₀alkynyl;
each R^{c} is at each occurrence independently selected from H, C₁-C₂₀alkyl, C₂-C₂₀alkenyl, and C₂-C₂₀alkynyl, each optionally substituted by one to six R^{b};
n is 0, 1, 2 or 3; and
m is 0, 1 or 2.

In an embodiment of (IA)-I, X is N—R^{1a}, and R^{1a} is an amine protecting group, e.g., tert-butyloxycarbonyl (Boc), N-carboxybenzyl (Cbz).

In a further embodiment of (IA)-I,
X is N—R^{1a}
R^{1a} is selected from C₁-C₁₀alkyl, C₃-C₁₀cycloalkyl, C₆-C₁₄aryl, C₇-C₂₀arylalkyl, and an amine protecting group, e.g., tert-butyloxycarbonyl (Boc), N-carboxybenzyl (Cbz); and
m is 0.

In a further embodiment of (IA)-I, X is N—R^{1a}; R^{1a} is an amine protecting group, e.g., tert-butyloxycarbonyl (Boc), N-carboxybenzyl (Cbz); and m is 0.

In the methods of the present disclosure, the engineered KRED polypeptides may be used to selectively catalyze the reduction of (IA) (substrate):

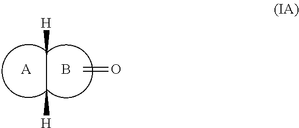

to (5s)-IA (product):

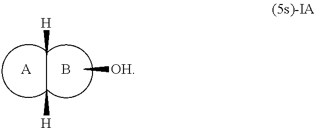

The engineered KRED polypeptides may be used to selectively catalyze the reduction of (IA)-I (substrate):

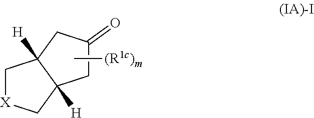

to (5s)-IA-I (product):

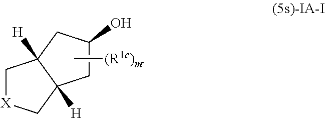

In the methods of the present disclosure, the engineered KRED polypeptides are used to selectively catalyze the reduction of a compound of formula (IA)-I to (IB)-I:

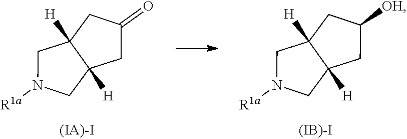

wherein R^{1a} is an amine protecting group. In a particular embodiment, R^{1a} is selected from tert-butyloxycarbonyl (Boc) and N-carboxybenzyl (Cbz).

In the methods of the present disclosure, the engineered KRED polypeptides are used to selectively catalyze the reduction of Compound 1 (substrate): tert-butyl rel-(3aR,6aS)-5-oxohexahydrocyclopenta[c]pyrrole-2(1H)-carboxylate:

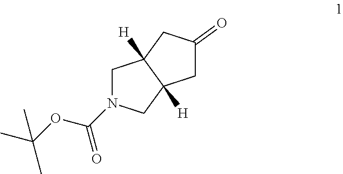 [SMILES: CC(C)(C)OC(=O)N1C[C@H]2CC(=O)C[C@H]2C1]

to (5s)-2 (product) tert-butyl rel-(3aR,5s,6aS)-5-hydroxyhexahydrocyclopenta[c]pyrrole-2(1H)-carboxylate:

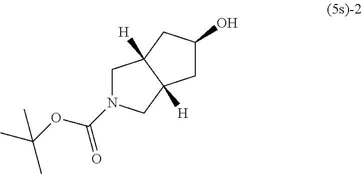 [SMILES: CC(C)(C)OC(=O)N1C[C@H]2C[C@H](O)C[C@H]2C1]

In some embodiments, the method for selectively reducing a compound of formula (IA) or (IA)-I (substrate) to (5s)-IA or (5s)-IA-I (product), including Compound I to (5s)-2, comprises contacting or incubating the substrate with a KRED polypeptide as disclosed herein under reaction conditions suitable for reducing or converting the substrate to the product compound. In some embodiments, the substrate is reduced to the (cis)-alcohol product (5s)-IA or (5s)-IA-I, e.g., (5s)-2, greater than about 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% diastereoisomeric excess over the corresponding (trans)-alcohol product (5r)-IA or (5r)-IA-I, e.g., (5r)-2:

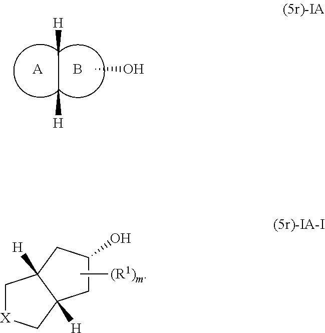

In some embodiments, the method for selectively reducing Compound 1 (substrate) to (5s)-2 (product) comprises contacting or incubating the substrate with an engineered KRED polypeptide as disclosed herein under reaction conditions suitable for reducing or converting the substrate to the product compound. In some embodiments, the substrate is reduced to the (cis) alcohol product ((5s)-2) greater than about 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% diastereoisomeric excess over the corresponding (trans) alcohol product (5r)-2: tert-butyl rel-(3aR,5r,6aS)-5-hydroxyhexahydrocyclopenta[c]pyrrole-2(1H)-carboxylate:

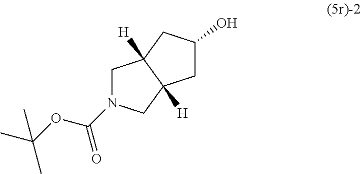 [SMILES: CC(C)(C)OC(=O)N1C[C@H]2C[C@@H](O)C[C@H]2C1]

In some embodiments, the substrate is reduced to the (cis) alcohol product ((5s)-2) greater than about 96% diastereoisomeric excess over the corresponding (trans) alcohol product ((5r)-2). In some embodiments, the substrate is reduced to the (cis) alcohol product ((5s)-2) greater than about 99% diastereoisomeric excess over the corresponding (trans) alcohol product ((5r)-2).

In some embodiments, the method for selectively reducing Compound 1 (substrate) to (5s)-2 (product) includes contacting or incubating the substrate with an engineered KRED polypeptide as disclosed herein under reaction conditions suitable for reducing or converting the substrate to the product compound at a greater stereoselectivity (% diastereomeric excess) and/or activity (% conversion) than that of a reference (e.g., parental) sequence (e.g., SEQ ID NO: 54, SEQ ID NO: 152, SEQ ID NO: 256, SEQ ID NO: 492). In some embodiments, the method for selectively reducing Compound 1 (Substrate) to (5s)-2 (Product) comprises contacting or incubating the Substrate with an engineered KRED polypeptide as disclosed herein under reaction conditions suitable for reducing or converting the Substrate to the Product compound at a greater stereoselectivity (% diastereomeric excess) and/or activity (% conversion) than that of SEQ ID NO: 54, SEQ ID NO: 152, SEQ ID NO: 256, and/or SEQ ID NO: 492.

As is known by those of skill in the art, ketoreductase-catalyzed reduction reactions typically require a cofactor. Reduction reactions catalyzed by the engineered KRED enzymes described herein also typically require a cofactor, although many embodiments of the engineered ketoreductases require far less cofactor than reactions catalyzed with wild-type KRED enzymes. As used herein, the term “cofactor” refers to a non-protein compound that operates in combination with a ketoreductase enzyme. Cofactors suitable for use with the engineered ketoreductase enzymes described herein include, but are not limited to, NADP⁺ (nicotinamide adenine dinucleotide phosphate), NADPH (the reduced form of NADP⁺), NAD⁺ (nicotinamide adenine dinucleotide) and NADH (the reduced form of NAD⁺). Generally, the reduced form of the cofactor is added to the reaction mixture. The reduced NAD(P)H form can be optionally regenerated from the oxidized NAD(P)⁺ form using a cofactor regeneration system. In some embodiments, the KRED enzyme polypeptides used in the methods provided herein are isolated and/or purified and the reduction reaction is carried out in the presence of a cofactor. In some embodiments, the cofactor is nicotinamide adenine dinucleotide (NADH) or nicotinamide adenine dinucleotide phosphate (NADPH).

In some embodiments, suitable reaction conditions may include a cofactor selected from the group consisting of NADP⁺, NADPH, NAD⁺ and NADH, at a concentration of about 0.05 g/L to about 0.1 g/L, 0.1 g/L to about 10 g/L, about 0.2 g/L to about 5 g/L, about 0.5 g/L to about 2.5 g/L. In some embodiments, the cofactor is NADH or NADPH. Accordingly, in some embodiments, suitable reaction conditions may include the cofactor NADH or NADPH at a concentration of about 0.05 g/L to about 10 g/L, 0.1 g/L to about 10 g/L, about 0.2 g/L to about 5 g/L, about 0.5 g/L to about 2.5 g/L. In some embodiments, the reaction conditions include about 10 g/L or less, about 5 g/L or less, about 2.5 g/L or less, about 1.0 g/L or less, about 0.5 g/L or less, about 0.05 g/L or less. In some embodiments, suitable reaction conditions for the methods provided herein include about 0.05 g/L NADP+ to about 1.0 g/L. In some embodiments, suitable reaction conditions for the methods provided herein include about 0.03 wt % to about 0.2 wt % cofactor (e.g., NADP+). In some embodiments, suitable reaction conditions for the methods provided herein include at least about 0.2 wt % cofactor (e.g., NADP+). In some embodiments, suitable reaction conditions for the methods provided herein include less than about 2 wt % cofactor (e.g., NADP+).

In some embodiments of the process (e.g., where whole cells or lysates are used), the cofactor is present naturally in the cell extract and does not need to be supplemented. In some embodiments of the process (e.g., using partially purified, or purified aldolase), the process may further include the step of adding cofactor to the enzymatic reaction mixture. In some embodiments, cofactor is added either at the beginning of the reaction and/or additional cofactor is added during the reaction.

The term “cofactor regeneration system” refers to a set of reactants that participate in a reaction that reduces the oxidized form of the cofactor (e.g., NADP⁺ to NADPH). Cofactors oxidized by the ketoreductase-catalyzed reduction of the keto substrate are regenerated in reduced form by the cofactor regeneration system. Cofactor regeneration systems comprise a stoichiometric reductant that is a source of reducing hydrogen equivalents and is capable of reducing the oxidized form of the cofactor. The cofactor regeneration system may further comprise a catalyst, for example an enzyme catalyst that catalyzes the reduction of the oxidized form of the cofactor by the reductant. Cofactor regeneration systems to regenerate NADH or NADPH from NAD⁺ or NADP⁺, respectively, are known in the art and may be used in the methods described herein.

Suitable exemplary cofactor regeneration systems that may be employed include, but are not limited to, glucose and glucose dehydrogenase, formate and formate dehydrogenase, glucose-6-phosphate and glucose-6-phosphate dehydrogenase, a secondary (e.g., isopropanol) alcohol and secondary alcohol dehydrogenase, phosphite and phosphite dehydrogenase, molecular hydrogen and hydrogenase, and the like. These systems may be used in combination with either NADP⁺/NADPH or NAD⁺/NADH as the cofactor. Electrochemical regeneration using hydrogenase may also be used as a cofactor regeneration system. See, e.g., U.S. Pat. Nos. 5,538,867 and 6,495,023, both of which are incorporated herein by reference. Chemical cofactor regeneration systems comprising a metal catalyst and a reducing agent (for example, molecular hydrogen or formate) are also suitable. See, e.g., International Pub. No. WO 2000/053731, which is incorporated herein by reference.

The terms “glucose dehydrogenase” and “GDH” are used interchangeably herein to refer to an NAD⁺ or NADP⁺-dependent enzyme that catalyzes the conversion of D-glucose and NAD⁺ or NADP⁺ to gluconic acid and NADH or NADPH, respectively. Equation (1), below, describes the glucose dehydrogenase-catalyzed reduction of NAD⁺ or NADP⁺ by glucose.

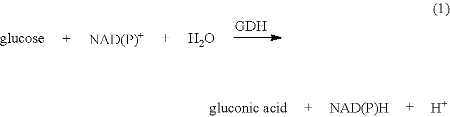

Glucose dehydrogenases that are suitable for use in the practice of the methods described herein include both naturally occurring glucose dehydrogenases, as well as non-naturally occurring glucose dehydrogenases. Naturally occurring glucose dehydrogenase encoding genes have been reported in the literature. For example, the Bacillus subtilis 61297 GDH gene was expressed in E. coli and was reported to exhibit the same physicochemical properties as the enzyme produced in its native host (Vasantha et al., 1983, Proc. Natl. Acad. Sci. USA 80:785). The gene sequence of the B. subtilis GDH gene, which corresponds to Genbank Acc. No. Ml 2276, was reported by Lampel et al., 1986, J. Bacteriol. 166:238-243, and in corrected form by Yamane et al., 1996, Microbiology 142:3047-3056 as Genbank Acc. No. D50453. Naturally occurring GDH genes also include those that encode the GDH from B. cereus ATCC 14579 (Nature, 2003, 423:87-91; Genbank Acc. No. AE017013) and B. megaterium (Eur. J. Biochem., 1988, 174:485-490, Genbank Acc. No. X12370; J. Ferment. Bioeng., 1990, 70:363-369, Genbank Acc. No. 01216270). Glucose dehydrogenases from Bacillus sp. are provided in International Pub. No. WO 2005/018579 as SEQ ID NOS: 10 and 12 (encoded by polynucleotide sequences corresponding to SEQ ID NOS: 9 and 11, respectively, in the International Publication), the disclosure of which is incorporated herein by reference.

Non-naturally occurring glucose dehydrogenases may be generated using known methods, such as, for example, mutagenesis, directed evolution, and the like. GDH enzymes having suitable activity, whether naturally occurring or non-naturally occurring, may be readily identified using the assay described in Example 4 of International Pub. No. WO 2005/018579, the disclosure of which is incorporated herein by reference. Exemplary non-naturally occurring glucose dehydrogenases are provided in International Pub. No. WO 2005/018579 as SEQ ID NOS: 62, 64, 66, 68, 122, 124, and 126. The polynucleotide sequences that encode them are provided in International Pub. No. WO 2005/018579 as SEQ ID NOS: 61, 63, 65, 67, 121, 123, and 125, respectively, the sequences of which are incorporated herein by reference. Additional non-naturally occurring glucose dehydrogenases that are suitable for use in the ketoreductase-catalyzed reduction reactions disclosed herein are provided in U.S. App. Pub. Nos. 2005/0095619 and 2005/0153417, the disclosures of which are incorporated herein by reference.

Glucose dehydrogenases employed in the ketoreductase-catalyzed reduction reactions described herein may exhibit an activity of at least about 10 μmol/min/mg and sometimes at least about 10² μmol/min/mg or about 10³ μmol/min/mg, up to about 10⁴ μmol/min/mg or higher in the assay described in Example 4 of International Pub. No. WO 2005/018579.

As disclosed herein and exemplified in the examples, the present disclosure contemplates a range of suitable reaction conditions that may be used in the process herein, including but not limited to pH, temperature, buffers, solvent systems, substrate loadings, mixtures of product stereoisomers, e.g., diastereoisomers, polypeptide loading, cofactor loading, pressure, and reaction time. Additional suitable reaction conditions for performing a method of enzymatically converting substrate compounds to a product compound using engineered KRED polypeptides described herein can be readily optimized by routine experimentation, which including but not limited to that the engineered KRED polypeptide is contacted with substrate compounds under experimental reaction conditions of varying concentration, pH, temperature, solvent conditions, and the product compound is detected, for example, using the methods described in the Examples provided herein.

The ketoreductase-catalyzed reduction reactions described herein are generally carried out in a solvent. Suitable solvents include water, organic solvents (e.g., ethyl acetate, isopropyl acetate, butyl acetate, methanol, ethanol, n-propanol, isopropanol, dimethyl sulfoxide, dimethylformamide, 1-octanol, hexane, heptane, octane, methyl t-butyl ether (MTBE), toluene, glycerol, polyethylene glycol, and the like), and ionic liquids (e.g., 1-ethyl-4-methylimidazolium tetrafluoroborate, 1-butyl-3-methylimidazolium tetrafluoroborate, 1-butyl-3-methylimidazolium hexafluorophosphate, and the like). In some embodiments, aqueous solvents, including water and aqueous co-solvent systems, are used.

In some embodiments, the solvent is present at a concentration of 0 to 500 g/L. In some embodiments, the solvent is present at a concentration of 100 to 200 g/L. In some embodiments, the solvent is present at a concentration of 0% to 100% v/v. In some embodiments, the solvent is present at a concentration of 20% to 40% v/v.

Exemplary aqueous co-solvent systems have water and one or more organic solvent. In general, an organic solvent component of an aqueous co-solvent system is selected such that it does not completely inactivate the ketoreductase enzyme. Appropriate co-solvent systems can be readily identified by measuring the enzymatic activity of the specified engineered ketoreductase enzyme with a defined substrate of interest in the candidate solvent system, utilizing an enzyme activity assay, such as those described herein.

The organic solvent component of an aqueous co-solvent system may be miscible with the aqueous component, providing a single liquid phase, or may be partly miscible or immiscible with the aqueous component, providing two liquid phases. Generally, when an aqueous co-solvent system is employed, it is selected to be biphasic, with water dispersed in an organic solvent, or vice-versa.

Generally, when an aqueous co-solvent system is utilized, it is desirable to select an organic solvent that can be readily separated from the aqueous phase. In general, the ratio of water to organic solvent in the co-solvent system is typically in the range of from about 90:10 to about 10:90 (v/v) organic solvent to water, and between 80:20 and 20:80 (v/v) organic solvent to water. The co-solvent system may be pre-formed prior to addition to the reaction mixture, or it may be formed in situ in the reaction vessel.

In some embodiments, the organic solvent in the aqueous co-solvent system is selected from ethyl acetate, isopropyl acetate, butyl acetate, methanol, ethanol, n-propanol, isopropanol, dimethyl sulfoxide, dimethylformamide, 1-octanol, hexane, heptane, octane, methyl t-butyl ether (MTBE), toluene, glycerol, polyethylene glycol, and the like), and ionic liquids (e.g., 1-ethyl-4-methylimidazolium tetrafluoroborate, 1-butyl-3-methylimidazolium tetrafluoroborate, 1-butyl-3-methylimidazolium hexafluorophosphate, and the like). In some embodiments the aqueous co-solvent system is water/isopropanol. In a particular embodiment, the isopropanol is present at a concentration of 20% to 40% v/v.

The aqueous solvent (water or aqueous co-solvent system) may be pH-buffered or unbuffered. Generally, the reduction can be carried out at a pH of about 10 or below, usually in the range of from about 5 to about 10. In some embodiments, the reduction is carried out at a pH of about 9 or below, usually in the range of from about 5 to about 9. In some embodiments, the reduction is carried out at a pH of about 8 or below, often in the range of from about 5 to about 8, and usually in the range of from about 6 to about 8. The reduction may also be carried out at a pH of about 7.8 or below, or 7.5 or below. Alternatively, the reduction may be carried out a neutral pH, i.e., about 7.

During the course of the reduction reactions, the pH of the reaction mixture may change. The pH of the reaction mixture may be maintained at a desired pH or within a desired pH range by the addition of an acid or a base during the course of the reaction. Alternatively, the pH may be controlled by using an aqueous solvent that comprises a buffer. Suitable buffers to maintain desired pH ranges are known in the art and include, for example, phosphate buffer, triethanolamine buffer, and the like. Combinations of buffering and acid or base addition may also be used.

When the glucose/glucose dehydrogenase cofactor regeneration system is employed, the co-production of gluconic acid (pKa=3.6), as represented in equation (1) causes the pH of the reaction mixture to drop if the resulting aqueous gluconic acid is not otherwise neutralized. The pH of the reaction mixture may be maintained at the desired level by standard buffering techniques, wherein the buffer neutralizes the gluconic acid up to the buffering capacity provided, or by the addition of a base concurrent with the course of the conversion. Combinations of buffering and base addition may also be used. Suitable buffers to maintain desired pH ranges are described above. Suitable bases for neutralization of gluconic acid are organic bases, for example amines, alkoxides and the like, and inorganic bases, for example, hydroxide salts (e.g., NaOH), carbonate salts (e.g., NaHCO₃), bicarbonate salts (e.g., K₂CO₃), basic phosphate salts (e.g., K₂HPO₄, Na₃PO₄), and the like. The addition of a base concurrent with the course of the conversion may be done manually while monitoring the reaction mixture pH or, more conveniently, by using an automatic titrator as a pH stat. A combination of partial buffering capacity and base addition can also be used for process control.

When base addition is employed to neutralize gluconic acid released during a ketoreductase-catalyzed reduction reaction, the progress of the conversion may be monitored by the amount of base added to maintain the pH. Typically, bases added to unbuffered or partially buffered reaction mixtures over the course of the reduction are added in aqueous solutions.

In some embodiments, the cofactor regenerating system can comprises a formate dehydrogenase. The terms “formate dehydrogenase” and “FDH” are used interchangeably herein to refer to an NAD⁺ or NADP⁺-dependent enzyme that catalyzes the conversion of formate and NAD⁺ or NADP⁺ to carbon dioxide and NADH or NADPH, respectively. Formate dehydrogenases that are suitable for use as cofactor regenerating systems in the ketoreductase-catalyzed reduction reactions as described herein include both naturally occurring formate dehydrogenases, as well as non-naturally occurring formate dehydrogenases. Formate dehydrogenases include those corresponding to SEQ ID NOS: 70 (Pseudomonas sp.) and 72 (Candida boidinii), which are encoded by polynucleotide sequences corresponding to SEQ ID NOS: 69 and 71, respectively, of International Pub. No. WO 2005/018579, the disclosure of which are incorporated herein by reference. Formate dehydrogenases employed in the methods described herein, whether naturally occurring or non-naturally occurring, may exhibit an activity of at least about 1 μmol/min/mg, sometimes at least about 10 μmol/min/mg, or at least about 10² μmol/min/mg, up to about 10³ μmol/min/mg or higher, and can be readily screened for activity using, for example, the assay described in Example 4 of International Pub. No. WO2005/018579.

As used herein, the term “formate” refers to formate anion (HCO₂), formic acid (HCO₂H), and mixtures thereof. Formate may be provided in the form of a salt, typically an alkali or ammonium salt (for example, HCO₂Na, KHCO₂NH₄, and the like), in the form of formic acid, typically aqueous formic acid, or mixtures thereof. Formic acid is a moderate acid. In aqueous solutions within several pH units of its pKa (pKa=3.7 in water) formate is present as both HCO₂⁻ and HCO₂H in equilibrium concentrations. At pH values above about pH 4, formate is predominantly present as HCO₂. When formate is provided as formic acid, the reaction mixture is typically buffered or made less acidic by adding a base to provide the desired pH, typically of about pH 5 or above. Suitable bases for neutralization of formic acid include, but are not limited to, organic bases, for example amines, alkoxides and the like, and inorganic bases, for example, hydroxide salts (e.g., NaOH), carbonate salts (e.g., NaHCO₃), bicarbonate salts (e.g., K₂CO₃), basic phosphate salts (e.g., K₂HPO₄, Na₃PO₄), and the like.

For pH values above about pH 5, at which formate is predominantly present as HCO₂⁻, Equation (2) below, describes the formate dehydrogenase-catalyzed reduction of NAD⁺ or NADP⁺ by formate.

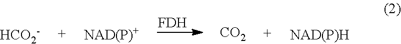

When formate and formate dehydrogenase are employed as the cofactor regeneration system, the pH of the reaction mixture may be maintained at the desired level by standard buffering techniques, wherein the buffer releases protons up to the buffering capacity provided, or by the addition of an acid concurrent with the course of the conversion. Suitable acids to add during the course of the reaction to maintain the pH include organic acids, for example carboxylic acids, sulfonicacids, phosphonic acids, and the like, mineral acids, for example hydrohalic acids (such as hydrochloric acid), sulfuric acid, phosphoric acid, and the like, acidic salts, for example dihydrogenphosphate salts (e.g., KH₂PO₄), bisulfate salts (e.g., NaHSO₄) and the like. Some embodiments utilize formic acid, whereby both the formate concentration and the pH of the solution are maintained.

When acid addition is employed to maintain the pH during a reduction reaction using the formate/formate dehydrogenase cofactor regeneration system, the progress of the conversion may be monitored by the amount of acid added to maintain the pH. Typically, acids added to unbuffered or partially buffered reaction mixtures over the course of conversion are added in aqueous solutions.

The terms “secondary alcohol dehydrogenase” and “sADH” are used interchangeably hereinto refer to an NAD⁺ or NADP⁺-dependent enzyme that catalyzes the conversion of a secondary alcohol and NAD⁺ or NADP⁺ to a ketone and NADH or NADPH, respectively. Equation (3) below describes the reduction of NAD⁺ or NADP⁺ by a secondary alcohol, illustrated by isopropanol.

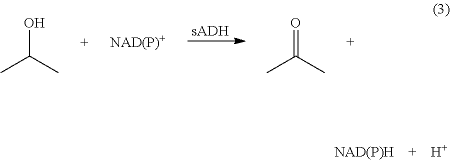

Secondary alcohol dehydrogenases that are suitable for use as cofactor regenerating systems in the ketoreductase-catalyzed reduction reactions described herein include both naturally occurring secondary alcohol dehydrogenases, as well as non-naturally occurring secondary alcohol dehydrogenases. Naturally occurring secondary alcohol dehydrogenases include known alcohol dehydrogenases from Thermoanerobium brockii, Rhodococcus etythropolis, Lactobacillus kefir, Lactobacillus minor and Lactobacillus brevis, and non-naturally occurring secondary alcohol dehydrogenases include engineered alcohol dehydrogenases derived therefrom. Secondary alcohol dehydrogenases employed in the methods described herein, whether naturally occurring or non-naturally occurring, may exhibit an activity of at least about 1 μmol/min/mg, sometimes at least about 10 μmol/min/mg, or at least about 10² μmol/min/mg, up to about 10³ μmol/min/mg or higher.

Suitable secondary alcohols include lower secondary alkanols and aryl-alkyl carbinols. Examples of lower secondary alcohols include isopropanol, 2-butanol, 3-methyl-2-butanol, 2-pentanol, 3-pentanol, 3,3-dimethyl-2-butanol, and the like. In one embodiment the secondary alcoholis isopropanol. Suitable aryl-akyl carbinols include unsubstituted and substituted 1-arylethanols.

In one embodiment, where oxidation of isopropanol to acetone is used for regeneration of NADH/NADPH, the reaction may be run at reduced pressure in such a manner that the acetone is removed from the reaction mixture.

When a secondary alcohol and secondary alcohol dehydrogenase are employed as the cofactor regeneration system, the resulting NAD⁺ or NADP+ is reduced by the coupled oxidation of the secondary alcohol to the ketone by the secondary alcohol dehydrogenase. Some engineered ketoreductases also have activity to dehydrogenate a secondary alcohol reductant. In some embodiments using secondary alcohol as reductant, the engineered ketoreductase and the secondary alcohol dehydrogenase are the same enzyme.

In carrying out embodiments of the ketoreductase-catalyzed reduction reactions described herein employing a cofactor regeneration system, either the oxidized or reduced form of the cofactor may be provided initially. As described above, the cofactor regeneration system converts oxidized cofactor to its reduced form, which is then utilized in the reduction of the ketoreductase substrate.

In some embodiments, cofactor regeneration systems are not used. For reduction reactions carried out without the use of a cofactor regenerating systems, the cofactor is added to the reaction mixture in reduced form. In some embodiments, suitable reaction conditions for the methods provided herein do not require GDH/Glucose cofactor recycling.

In some embodiments, the method is carried out with whole cells that express the ketoreductase enzyme, or an extract or lysate of such cells. When the process is carried out using whole cells of the host organism, the whole cell may natively provide the cofactor. Alternatively or in combination, the cell may natively or recombinantly provide the glucose dehydrogenase.

In carrying out the stereoselective reduction reactions described herein, the engineered KRED enzyme, and any enzymes comprising the optional cofactor regeneration system, may be added to the reaction mixture in the form of the purified enzymes, whole cells transformed with gene(s) encoding the enzymes, and/or cell extracts and/or lysates of such cells. The gene(s) encoding the engineered ketoreductase enzyme and the optional cofactor regeneration enzymes can be transformed into host cells separately or together into the same host cell. For example, in some embodiments one set of host cells can be transformed with gene(s) encoding the engineered KRED enzyme and another set can be transformed with gene(s) encoding the cofactor regeneration enzymes. Both sets of transformed cells can be utilized together in the reaction mixture in the form of whole cells, or in the form of lysates or extracts derived therefrom. In other embodiments, a host cell can be transformed with gene(s) encoding both the engineered ketoreductase enzyme and the cofactor regeneration enzymes.

Whole cells transformed with gene(s) encoding the engineered KRED enzyme and/or the optional cofactor regeneration enzymes, or cell extracts and/or lysates thereof, may be employed in a variety of different forms, including solid (e.g., lyophilized, spray-dried, and the like) or semisolid (e.g., a crude paste).

The cell extracts or cell lysates may be partially purified by precipitation (ammonium sulfate, polyethyleneimine, heat treatment or the like) followed by a desalting procedure prior to lyophilization (e.g., ultrafiltration, dialysis, and the like). Any of the cell preparations may be stabilized by crosslinking using known crosslinking agents, such as, for example, glutaraldehyde or immobilization to a solid phase material (e.g., Eupergit C, resin and the like).

In any of the embodiments of the process disclosed herein, the reaction is performed under suitable reaction conditions described herein, wherein the engineered ketoreductase polypeptide is immobilized to a solid support, such as a membrane, resin, solid carrier, or other solid phase material. A solid support can be composed of organic polymers such as microcrystalline cellulose, polystyrene, polyethylene, polypropylene, polyfluoroethylene, polyethyleneoxy, polymethacrylate, and polyacrylamide, as well as co-polymers and grafts thereof. A solid support can also be inorganic, such as celite (diatomaceous earth), glass, silica, controlled pore glass (CPG), reverse phase silica or metal, such as gold or platinum. The configuration of a solid support can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location. Solid supports useful for immobilizing the engineered ketoreductase enzyme for carrying out the reaction include but are not limited to beads or resins such as polymethacrylate, e.g., polymethacrylates with epoxy functional groups, polymethacrylates with amino functional groups, polymethacrylates, styrene/DVB copolymer or polymethacrylates with octadecyl functional groups. In a particular embodiment, the solid support is a bead or resin comprising polymethacrylate.

Exemplary solid supports include, but are not limited to, chitosan beads, Eupergit C, IB-150, IB-350, IB-C435, IB-A369, IB-A161, IB-A171, IBS500, IB-S861, SEPABEADS (Mitsubishi), e.g., Sepabeads EC-EP, Sepabeads EC-HFA, Sepabeads EC-HG, Sepabeads EC-BU, Sepabeads EC-OD, Sepabeads EC-CM, Sepabeads EC-IDA, Sepabeads EC-EA, Sepabeads EC-HA, Sepabeads EC-QA, Sepabeads EXE, Sepabeads EXA, Dilbeads-TA, Amberzyme Oxirane, Amberlite XAD-7HP, Amberlite FPA98Cl, Amberlite IRA958C1, Amberlite IRA67, Amberlite FPA90C1, Amberlite FPA40C1, Amberlite XAD18, Accurel EP100, ECR8206F/5730, ECR8206/5803, ECR8206M/5749, ReliZyme EP403, ReliZymeEP113, Lewatit VP OC 1600, Diaion WA20, Diaion WA21J, Diaion WA30, Dowex 66, Diaion HPA-25L, Lewatit VP OC 1064 MD PH, Lewatit VP OC 1163, Lifetech ECR8304F. Lifetech ECR8309F, Lifetech ECR8315F, Lifetech ECR8204F, Lifetech ECR8285, Lifetech ECR1090M, Lifetech ECR1030M, Lifetech ECR8806M, Chromalite (MAM2/F) D6591, Chromalite MIDA/M, Chromalite MIDA/M/Fe, Chromalite MIDA/M/Co, Chromalite MIDA/M/Ni, Chromalite MIDA/M/Cu and Chromalite MIDA/M/Zn.

In any of the embodiments of the process disclosed herein, wherein an engineered polypeptide is expressed in the form of a secreted polypeptide, a culture medium containing the secreted polypeptide can be used in the process herein.

In any of the embodiments of the process disclosed herein, the solid reactants (e.g., enzyme, salts, etc.) may be provided to the reaction in a variety of different forms, including powder (e.g., lyophilized, spray dried, and the like), solution, emulsion, suspension, and the like. The reactants can be readily lyophilized or spray dried using methods and equipment that are known to those having ordinary skill in the art. For example, the protein solution can be frozen at −80 °C in small aliquots, then added to a prechilled lyophilization chamber, followed by the application of a vacuum. After the removal of water from the samples, the temperature is typically raised to 4 °C for two hours before release of the vacuum and retrieval of the lyophilized samples.

The quantities of reactants used in the reduction reaction will generally vary depending on the quantities of product desired, and concomitantly the amount of ketoreductase substrate employed. The following guidelines can be used to determine the amounts of ketoreductase, cofactor, and optional cofactor regeneration system to use. Generally, keto substrates can be employed at a concentration of about 5 to 150 grams/liter using from about 50 mg to about 5 g of ketoreductase and about 10 mg to about 150 mg of cofactor.

In some embodiments, suitable reaction conditions for the methods provided herein include about 5 g/L to about 150 g/L of Substrate. In some embodiments, suitable reaction conditions for the methods provided herein use a concentration of the substrate of at least about 5 g/L, at least about 10 g/L, at least about 20 g/L, at least about 50 g/L, at least about 100 g/L, or at least about 150 g/L. In some embodiments, the concentration of the ketoreductase is less than about 5 g/L. In some embodiments, the concentration of the ketoreductase is at least 3 g/L. In some embodiments, suitable reaction conditions for the methods provided herein include ketoreductase loading of at least about 1 wt %. In some embodiments, suitable reaction conditions for the methods provided herein include ketoreductase loading of less than 3 wt %. In some embodiments, suitable reaction conditions for the methods provided herein include ketoreductase loading of about 0.8 wt % and 150 g/L ketone substrate.

Those having ordinary skill in the art will readily understand how to vary these quantities to tailor them to the desired level of productivity and scale of production. Appropriate quantities of optional cofactor regeneration system may be readily determined by routine experimentation based on the amount of cofactor and/or ketoreductase utilized. In general, the reductant (e.g., glucose, formate, and isopropanol) is utilized at levels above the equimolar level of ketoreductase substrate to achieve essentially complete or near complete conversion of the ketoreductase substrate.

In any of the embodiments of the process disclosed herein, the order of addition of reactants is not critical. The reactants may be added together at the same time to a solvent (e.g., monophasic solvent, biphasic aqueous co-solvent system, and the like), or alternatively, some of the reactants may be added separately, and some together at different time points. For example, the cofactor regeneration system, cofactor, ketoreductase, and ketoreductase substrate may be added first to the solvent.

For improved mixing efficiency when an aqueous co-solvent system is used, the cofactor regeneration system, ketoreductase, and cofactor may be added and mixed into the aqueous phase first. The organic phase may then be added and mixed in, followed by addition of the ketoreductase substrate. Alternatively, the ketoreductase substrate may be premixed in the organic phase, prior to addition to the aqueous phase

Suitable conditions for carrying out the ketoreductase-catalyzed reduction reactions described herein include a wide variety of conditions which can be readily optimized by routine experimentation that includes, but is not limited to, contacting the engineered ketoreductase enzyme and substrate at an experimental pH and temperature and detecting product, for example, using the methods described in the Examples provided herein.

The ketoreductase catalyzed reduction is typically carried out at a temperature in the range of from about 15 °C to about 75 °C. For some embodiments, the reaction is carried out at a temperature in the range of from about 20 °C to about 55 °C. In still other embodiments, it is carried out at a temperature in the range of from about 20 °C to about 45 °C. In some embodiments, it is carried out at 40 °C. The reaction may also be carried out under ambient conditions.

The reduction reaction is generally allowed to proceed until essentially complete, or near complete, reduction of substrate is obtained. In some embodiments, isopropanol (iPrOH) is used as the solvent. In some embodiments, suitable reaction conditions for the methods provided herein include up to 40% (v/v) of iPrOH. Optionally, the removal of acetone formed in the oxidation of iPrOH is performed. Further, addition of iPrOH can facilitate the reaction to completion. Reduction of substrate to product can be monitored using known methods by detecting substrate and/or product. Suitable methods include gas chromatography, HPLC, and the like. Conversion yields of the alcohol reduction product generated in the reaction mixture are generally greater than about 50%, but may also be greater than about 60%, 70%, 80%, or 90%, and are often greater than about 97%. In some embodiments, conversion yields of the alcohol reduction product generated in the reaction mixture are generally greater than about 85% up to full conversion.

Whether carrying out the method with whole cells, cell extracts or purified ketoreductase enzymes, a single ketoreductase enzyme may be used or, alternatively, mixtures of two or more ketoreductase enzymes may be used.

Suitable reaction conditions can include a combination of reaction parameters that provide for the biocatalytic conversion of the substrate compound to its corresponding product compound. Accordingly, in some embodiments of the process, the combination of reaction parameters comprises one or more of the following: i) substrate loading, e.g., Compound 1 loading of about 5 g/L to about 150 g/L; ii) engineered enzyme polypeptide concentration of at least about 3 g/L or at least about 1 wt % or less than about 3 wt %; iii) cofactor/cofactor loading, e.g., NADP+ cofactor loading of about 10 mg to about 150 mg or at least about 0.1 wt % NADP+ cofactor or less than about 2 wt % NADP+ cofactor; iv) co-solvent concentration of about 20% (v/v) to about 60% (v/v) (e.g., up to 40% (v/v) of isopropanol); v) a temperature of about 15 to 75 °C, e.g., 20 to 55 °C, e.g., 20 to 45 °C, e.g., 40 °C; vi) a pH of 5.0 to 10.0, e.g., 7.5; and vii) a reaction time of up to 24 hours.

The combination of reaction parameters may also affect reaction time. In some embodiments, at least about 90% of the substrate is reduced to the product in less than about 20 hours. In some embodiments, at least about 95% of the substrate is reduced to the product in less than about 24 hours.

The methods of performing an enzymatic reaction may comprise the further step of isolating the product of the enzymatic reaction. In particular, this step is performed after completion of the enzymatic reaction. The product is in particular separated from one or more, in particular essentially all of the other components of the reaction mixture. For example, the product is separated from the remaining substrate, side products, the enzyme, and/or organic solvents. Isolation of the product may be achieved by means and techniques known in the art, including for example evaporation of solvents, aggregation or crystallization and filtration, phase separation, chromatographic separation and others.

The present disclosure also provides a process for synthesizing 6-((S)-2-((3aR,5R,6aS)-5-(2-fluorophenoxy)hexahydrocyclopenta[c]pyrrol-2(1H)-yl)-1-hydroxyethyl)pyridin-3-ol, in free form or in pharmaceutically acceptable salt form, of formula (IC)

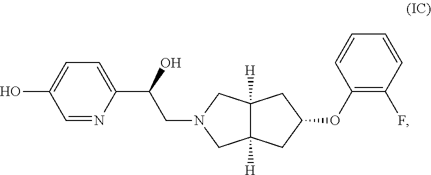 [SMILES: Oc1ccc([C@@H](O)CN2C[C@H]3C[C@H](Oc4ccccc4F)C[C@H]3C2)nc1]

the process comprising the step of contacting a compound of formula (IA)-I with the KRED polypeptide according to the present disclosure, to obtain a compound of formula (IB)-I

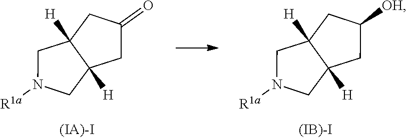

wherein R^{1a} is an amine protecting group. In a particular embodiment, R^{1a} is selected from tert-butyloxycarbonyl (Boc) and N-carboxybenzyl (Cbz).

In a particular embodiment, the process comprises the step of contacting Compound 1 with the KRED polypeptide according to the present disclosure, to obtain tert-butyl rel-(3aR,5s,6aS)-5-hydroxyhexahydrocyclopenta[c]pyrrole-2(1H)-carboxylate:

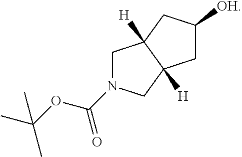 [SMILES: CC(C)(C)OC(=O)N1C[C@H]2C[C@H](O)C[C@H]2C1]

In a further aspect, there is provided a compound of formula

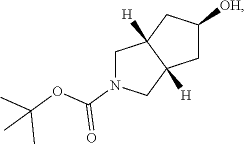 [SMILES: CC(C)(C)OC(=O)N1C[C@H]2C[C@H](O)C[C@H]2C1]

or a salt thereof.

In a further aspect, the is provided the use of a compound of formula

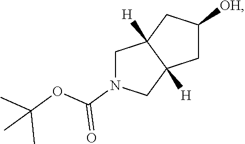 [SMILES: CC(C)(C)OC(=O)N1C[C@H]2C[C@H](O)C[C@H]2C1]

or a salt thereof, in the preparation of 6-((S)-2-((3aR,5R,6aS)-5-(2-fluorophenoxy)hexahydrocyclopenta[c]pyrrol-2(1H)-yl)-1-hydroxyethyl)pyridin-3-ol, in free form or in pharmaceutically acceptable salt form.

Compound (IC) can be synthesized from tert-butyl rel-(3aR,5s,6aS)-5-hydroxyhexahydrocyclopenta[c]pyrrole-2(1H)-carboxylate (or (5s)-2) by known synthetic procedures in the art. In particular, Compound (IC) can be synthesized from tert-butyl rel-(3aR,5s,6aS)-5-hydroxyhexahydrocyclopenta[c]pyrrole-2(1H)-carboxylate according to the methods and procedures disclosed in WO 2016/049165 A1.

### Compositions, Kits, and Administration

The present disclosure provides compositions comprising any of the engineered KRED polypeptides, polynucleotides, expression vectors, and/or host cells, as described herein. Compositions described herein can be prepared by any method known in the art of pharmacology. In general, such preparatory methods include the steps of bringing the “active ingredient” (e.g., the engineered KRED polypeptide) into association with a carrier and/or one or more other accessory ingredients. Compositions can be prepared, packaged, and/or sold in bulk. Relative amounts of the active ingredient and/or any additional ingredients in a composition of the invention will vary, depending upon the use.

It will be also appreciated that any of the compositions as described herein can include one or more additional agents. In some embodiments, the composition comprises the structural formula of a substrate (e.g., tert-butyl rel-(3aR,6aS)-5-oxohexahydrocyclopenta[c]pyrrole-2(1H)-carboxylate (Compound 1)) and/or the compound of the product (e.g., (5s)-2). For, example, the composition can include the structural formula of Compound 1 and/or the compound of (5s)-2. In some embodiments, the composition can include a cofactor, such as NAD(P)H.

Also encompassed by the disclosure are kits. The kits of the invention may include any of the engineered KRED polypeptides, polynucleotides, expression vectors, and/or host cells, or compositions thereof, as described herein. In some embodiments, the kit includes any of the engineered KRED polypeptides, a substrate (e.g., tert-butyl rel-(3aR,6aS)-5-oxohexahydrocyclopenta[c]pyrrole-2(1H)-carboxylate (Compound 1)), and a cofactor. In some embodiments, the cofactor is nicotinamide adenine dinucleotide (NADH) or nicotinamide adenine dinucleotide phosphate (NADPH). In some embodiments, the kit includes an immobilized KRED polypeptide.

The kits may be useful for performing any of the methods as described herein, such as producing and/or using any of the engineered KRED polypeptides, polynucleotides, expression vectors, and/or host cells, or compositions thereof, as described herein. In some embodiments, the kit is used in a method for reducing tert-butyl rel-(3aR,6aS)-5-oxohexahydrocyclopenta[c]pyrrole-2(1H)-carboxylate (substrate) to tert-butyl rel-(3aR,5s,6aS)-5-hydroxyhexahydrocyclopenta[c]pyrrole-2(1H)-carboxylate (product). In some embodiments, the kit is used in a method for reversing the diastereoselectivity of a ketoreductase polypeptide. In some embodiments, the kit is used in a method for increasing the diastereoselectivity of a ketoreductase polypeptide.

The kits provided herein may comprise one or more containers (e.g., a vial, ampule, bottle, syringe, and/or dispenser package, or other suitable container). The kits provided herein may also comprise written instructions for producing or using any of the engineered KRED polypeptides, polynucleotides, expression vectors, and/or host cells, or compositions thereof, as described herein.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Expression Strain Description

A polypeptide from a prior ketoreductase (KRED) panel (SEQ ID NO: 2) was selected with suitable initial activity/selectivity toward the formation of compound (5s)-2, and that variant was used as the “backbone” for the first round of evolution. A second polypeptide from the prior ketoreductase panel (SEQ ID NO: 54) was found more appropriate for evolution with regards to its selectivity towards the formation of compound (5s)-2 at low conversion. This variant was used as the backbone for the second round of evolution.

The parent genes for the KREDs (Round 1 and Round 2 backbone) used to produce the variants of the present invention were codon optimized for expression in E. coli and cloned into the vector pCKI 10900 (vector depicted as FIG. 3 in U.S. Patent App. Pub. 20060195947, which is hereby incorporated by reference herein) under the control of a lac promoter. The expression vector also contained the P15a origin of replication and the chloramphenicol (CAM) resistance 20 gene. Resulting plasmids were transformed into E. coli W3110 (fhu-) using standard methods.

Multiple rounds of directed evolution of the KRED gene in the pCKI 10900 plasmid were carried out using the gene encoding the most improved polypeptide from each round as the parent backbone sequence for the subsequent round of evolution. The resulting exemplary engineered ketoreductase polypeptide sequences and specific mutations and relative activities of the present invention are listed in Tables 4, 5, 6 and 8.

### Example 2: Preparation of Cell Pellets (from Growth to Lysis)

E. coli W3110 (fhu-) cells were transformed with the pCKI 10900 plasmid containing the KRED-encoding genes and plated on LB agar plates containing 1% glucose and 30 μg/mL CAM, and grown overnight at 37 °C. Monoclonal colonies were picked and inoculated into 180 μL LB containing 1% glucose and 30 μg/mL CAM in 96-well shallow-well microtiter plates. The plates were sealed with O₂-permeable seals and cultures were grown overnight at 30 °C, 200 rpm and 85% RH. Then, 10 μL of each of the cell cultures were transferred into the wells of 96-well deep-well plates containing 390 μL Terrific Broth (TB) and 30 μg/mL CAM. The deep-well plates were sealed with O₂-permeable seals and incubated at 30 °C, 250 rpm and 85% humidity until an optical density at 600 nm (OD₆₀₀) of 0.6-0.8 was reached. Expression of the KRED gene was induced by the addition of isopropyl-β-D-thiogalactoside (IPTG) to a final concentration of 1 mM and incubated overnight at 30 °C, 250 rpm, and 85% humidity. The cells were then pelleted using centrifugation at 4000 rpm for 10 mM. The supernatants were discarded and the pellets frozen at −80 °C prior to lysis.

### Example 3: Lysis and Preparation of Clarified Lysate

Frozen pellets prepared as specified in Example 2 were lysed with 200 μL lysis buffer containing either 100 mM TEoA (Triethanolamine chloride) or NaPi (Sodium phosphate) buffer, pH 7.0, 1 mg/mL lysozyme, 0.5 mg/mL PMBS, 0.2 U/mL DNAse, 10 mM MgSO₄. The lysis mixture was shaken at RT for 2.5 hours. The plate was then centrifuged for 10 min at 4000 rpm and 4 °C. The supernatants were then used in biocatalytic reactions as clarified lysates, in experiments described below to determine the activity levels.

### Example 4: Preparation of Shake Flask Powder

A shake-flask procedure was used to generate engineered polypeptide powders used in high-throughput activity assays. A single microbial colony of E. coli containing a plasmid with the KRED gene of interest was used to inoculate 50 mL Luria Bertani (LB) broth containing 30 μg/mL chloramphenicol and 1% glucose. Cells were grown overnight (at least 16 hrs) in an incubator at 30 °C with shaking at 250 rpm. The culture was diluted into 250 mL TB in a 1 L flask and grown to an OD₆₀₀ of 0.2 and allowed to grow at 30 °C, 250 rpm. Expression of the ketoreductase gene was induced with 1 mM IPTG when the OD₆₀₀ of the culture was between 0.6 to 0.8 and incubation was continued overnight (at least 16 hrs). Cells were harvested by centrifugation (5000 rpm, 15 min, 4 °C) and the supernatant discarded. The cell pellet was resuspended in 50 mL of cold (4 °C) 100 mM triethanolamine (chloride) buffer, pH 7.0, and harvested by centrifugation as above. The washed cells were resuspended in 30 mL of cold triethanolamine (chloride) buffer and passed through a microfluidizer. Cell debris was removed by centrifugation (9000 rpm, 45 min., 4 °C). The clear lysate supernatant was collected and stored at −20 °C. Lyophilization of frozen, clear lysate provided a dry powder of crude KRED enzyme.

### Example 5: Analytical Method for Activity and Selectivity Evaluation

An UHPLC method with UV-detection was developed to analyze the conversion of Compound 1 to compounds (5s)-2 and (5r)-2 (see FIG. 1). The conversion is expressed as a percentage and is calculated as follows:

$$\% Conversion = \frac{( area [ ( 5 s ) - 2 ] )}{( area of [ ( 5 s - 2 ) ] ) + ( area of [ ( 5 r - 2 ) ] ) + ( area of 1 * RRF )} * 100$$

RRF=1.1 corresponding to the Relative Response Factor between the substrate and the products. The RRF value was determined in the concentration range from 0.2 mg/mL to 2.0 mg/mL of substrate 1 and product 2.

The product compounds (5s)-2 and (5r)-2 were diastereomers and consequently were well separated with the described analytical method. The diastereomeric excess (de) expressed as a percentage was calculated as follows:

$$\% de = \frac{( area [ ( 5 s ) - 2 ] - area [ ( 5 r ) - 2 ] )}{( area [ ( 5 s ) - 2 ] + area [ ( 5 r ) - 2 ] )} * 100$$

Data described in the Examples were collected using the analytical method in Table 3. The method provided herein finds use in analyzing the variants produced using the present invention. However, the present invention is not intended to be limited to the methods described herein, as there are other suitable methods known in the art that are applicable to the analysis of the variants provided herein and/or produced using the methods provided herein.

TABLE 3
Analytical Method

| Instrument | Agilent Technologies 1290 Infinity | |
|---|---|---|
| | II with UV detection | |
| Column | Agilent Zorbax Eclipse Plus | |
| | C18 (Rapid Resolution HD) | |
| | 50 × 3.0 mm, 1.8 μm | |
| Mobile Phase | A: Water/Acetonitrile (95:5) + 0.05% TFA | |
| | B: Water/Acetonitrile (5:95) + 0.05% TFA | |
| Gradient | Time (min.) | Phase B (%) |
| | 0.00 | 22 |
| | 1.10 | 22 |
| | 1.15 | 80 |
| | 1.60 | 80 |
| | 1.65 | 22 |
| | 2.00 | 22 |

| | |
|---|---|
| Flow Rate | 2.5 mL/min |
| Run Time | 2.00 minutes |
| Compound | Compound (5s)-2: 0.73 min. |
| elution | Compound (5r)-2: 0.84 min. |
| | Compound 1: 0.93 min. |
| Column Temperature | 40 °C |
| Injection volume | 1 μL |
| Detection | UV 210 nm |

### Example 6: Evaluation of a Collection of KREDs for the Reduction of Compound 1

A collection of Ketoreductases (KREDs) was screened using 250 μL reaction volumes in deep-well Costar plates with each well containing 40 mM Triethanol amine (TEoA) buffer pH 7.0, 5 g/L Compound 1, 1 g/L NADP+, 20% isopropanol (v/v) and 40% (v/v) enzyme lysate. Sealed plates were incubated at room temperature with vigorous shaking at 850 rpm in Infors incubator. After 24 hours incubation, reactions were quenched by addition of 750 μL of acetonitrile. Plates were resealed and vigorously shaken for 10 minutes at room temperature at 800 rpm in plate shaker and subsequently centrifuged for 10 minutes at 4000 g. 200 μL of supernatant was transferred into microtiter plates (V-bottom Greiner plates), sealed with aluminum foil and subjected to HPLC analysis using the method described in Example 5. KRED with SEQ ID NO: 2 (nucleic acid sequence of SEQ ID NO: 1) was identified as the highest performing enzyme towards the production of compound (5s)-2.

### Example 7: KRED Improvements Over SEQ ID NO: 2 for Diastereoselective Production of Compound (5s)-2 (Round 1)

An engineered KRED, SEQ ID NO: 2 (nucleic acid sequence SEQ ID NO: 1) was selected as the parent enzyme for the first round of directed evolution. Libraries of engineered genes were produced using well-established techniques (e.g., saturation mutagenesis, and recombination of mutations identified as potentially beneficial in variants screened in panels of enzymes). The enzymes encoded by each gene were produced in HTP as described in Example 2, and the clarified lysates were generated as described in Example 3.

Each 200 μL reaction was carried out in 96-well deep-well format (2 mL volume) with 0.625-2.5% (v/v) of clarified lysate, 10 g/L Compound 1, 20% (v/v) isopropanol, 40 mM TEoA buffer pH 7.0, 0.1 g/L NADP⁺. The plates were sealed and agitated at 30 °C at 600 rpm in Infors shaker for 24 hours.

Reactions were quenched by the addition of 800 μL acetonitrile into each well of the reactions plate, sealed and shaken for 15 minutes. The plates were then centrifuged at 4000 rpm for 10 minutes, 200 μL of the supernatant transferred to analytical plates and subjected to achiral HPLC analysis using method described in Example 5.

The activity of each variant was calculated as the percent conversion of the products formed based on peak area [((5s)-2+(5r)-2)/(Compound 1+(5s)-2+(5r)-2)*100]. The selectivity of each variant was calculated as the percent diastereomeric excess (d.e.) of the desired product (5s)-2 formed [((5s)-2−(5r)-2)/((5s)-2+(5r)-2)*100] based on peak area. In addition, the amount of desired product (5s)-2 was reported as HPLC peak area since the resulting amount if a convolution of the conversion and selectivity. For all reported values, the fold improvement over SEQ ID NO: 2 was calculated. As the parent SEQ ID NO: 2 had a negative % d.e. value, the inverse of the fold improvement was reported for clarity in order to report an improvement of the enzymatic activity towards the desired product (5s)-2 as a positive value greater than 1. The results are provided in Table 4.

TABLE 4
Engineered variants relative to SEQ ID NO: 2 (Round 1)

| | SEQ ID | Amino Acid | | FIOP | |
|---|---|---|---|---|---|
| SEQ ID | NO: | Differences | | % de of | FIOP |
| NO: | (amino | relative to | FIOP % | desired | desired |
| (nucleotide) | acid) | SEQ ID NO: 2 | conversion | product | product |
| 3 | 4 | T152Q | − | ++ | + |
| 5 | 6 | T152L | − | ++ | ++ |
| 7 | 8 | A94L | +++ | + | ++++ |
| 9 | 10 | T152R | − | ++ | +++ |
| 11 | 12 | Q206A | +++ | − | + |
| 13 | 14 | F147V | +++ | + | +++ |
| 15 | 16 | P194C | +++ | − | + |
| 17 | 18 | Q206L | ++ | − | + |
| 19 | 20 | S96A | ++ | ++ | ++++ |
| 21 | 22 | D198Q | ++ | − | +++ |
| 23 | 24 | S96G | +++ | + | ++++ |
| 25 | 26 | H40R | ++ | + | +++ |
| 27 | 28 | G7S | − | ++ | + |
| 29 | 30 | G7S/S96Y/ | − | +++ | + |
| | | E145S/F147L/ | | | |
| | | A190P/K211L | | | |
| 31 | 32 | G7S/H40R/ | − | +++ | + |
| | | S96Y/R108H/ | | | |
| | | E145S/F147L/ | | | |
| | | A190P/K211L | | | |
| 33 | 34 | G7S/H40K/ | − | ++ | + |
| | | G117S/E145S/ | | | |
| | | F147L/A190P/ | | | |
| | | K211L | | | |
| 35 | 36 | A190P/Q206N | + | + | +++ |
| 37 | 38 | F147L | + | + | + |
| 39 | 40 | A190V/L196I/ | − | +++ | ++ |
| | | Q206W | | | |
| 41 | 42 | H40R/F147L/ | + | − | + |
| | | A190C/L196I | | | |
| 43 | 44 | H40K/A190C/ | + | + | + |
| | | L196I/Q206N | | | |
| 45 | 46 | Q206N | + | ++ | +++ |
| 47 | 48 | H40R/A190C/ | + | + | +++ |
| | | L196I/Q206N | | | |
| 49 | 50 | H40K/A190P/ | + | + | ++ |
| | | Q206N | | | |
| 51 | 52 | H40R/F147L/ | ++ | + | ++ |
| | | A190P/L196I/ | | | |
| | | L199V/Q206N | | | |

Fold improvement over positive control (FIOP) % conversion-Levels of increased activity were determined relative to the reference polypeptide of SEQ ID NO: 2 and defined as follows: “−” 0 to 1.00, “+” > 1.00 “++” > 1.50, “+++” > 1.75.
Fold improvement over positive control (FIOP) % of desired product-Levels of
increased selectivity were determined relative to the reference polypeptide of SEQ ID NO: 2 and defined as follows: “−” 0 to 1.01, “+” > 1.01, “++” > 1.05, “+++” > 1.10.
Fold improvement over positive control (FIOP) of desired product-Levels of increased activity were determined relative to the reference polypeptide of SEQ ID NO: 2 and defined as follows: “+” 1.00 to 1.50), “++” > 1.50, “+++” > 2.00, “++++” > 3.00.

The best performing variants were prepared as shake flask powders and evaluated under two conditions: 10 g/L Compound 1 with 20% isopropanol (v/v) or 20 g/L Compound 1 with 40% isopropanol (v/v). Reactions were prepared in 200 μL volume and both conditions contained 1 g/L NADP+ and 0.1 mM TEoA buffer pH 7.0. 2-fold serial dilutions of enzyme shake flask powder with highest final concentration of 4 g/L were added to the reaction mixtures. Reactions were incubated at room temperature for 24 hours with shaking at 600 rpm, then quenched with acetonitrile and centrifuged at 4000 rpm for 10 minutes. 100 μL of the supernatant transferred into Greiner microtiter plates before being subjected to HPLC analysis. Percent conversion and percent diastereomeric excess (% de) were calculated as described above. The % de dose curve showed an increase of desired product formation with increasing enzyme concentration, suggesting a dynamic resolution of compound (5s)-2.

### Example 8: Evaluation of a Collection of KREDs for the Reduction of Compound 1 Under Lower Lysate Concentration

A collection of Ketoreductases (KREDs) was screened to identify an exemplary enzyme similar to Example 6. Screening was performed in 250 μL reaction volumes in Costar plates with each well containing 40 mM Triethanol amine (TEoA) buffer pH 7.0, 5 g/L Compound 1, 1 g/L NADP⁺, 20% isopropanol (v/v) and either 40% (v/v) enzyme lysate, 5% (v/v) lysate or 0.625% (v/v) lysate. Sealed plates were incubated at room temperature with vigorous shaking at 850 rpm in Infors incubator. After 24 hours incubation, reactions were quenched by addition of 750 μL of acetonitrile. Plates were resealed and incubated for 10 minutes at room temperature with vigorous shaking and subsequently centrifuged for 2 minutes at 3220 g. 200 μL of supernatant were transferred into microtiter plates (V-bottom Greiner plates), sealed with aluminum foil and subjected to HPLC analysis using the method described in Example 5. Percent conversion and percent diastereomeric excess were calculated at all tested lysate concentrations and the constant % de was used as the main selection criteria to identify the optimal enzymes. KRED with SEQ ID NO: 54 (nucleic acid sequence SEQ ID NO: 53) was identified as the best performing enzyme towards the production of compound (5s)-2 at varying lysate concentrations.

### Example 9: KRED Improvements Over SEQ ID NO: 54 for Diastereoselective Production of Compound (5s)-2 (Round 2)

SEQ ID NO: 54 (nucleic acid sequence SEQ ID NO: 53) was selected as the parent enzyme for the second round of directed evolution. Libraries of engineered genes were produced using well-established techniques (e.g., saturation mutagenesis, and recombination of mutations identified as potentially beneficial in previous round of evolution). The enzymes encoded by each gene were produced in HTP as described in Example 2, and the clarified lysates were generated as described in Example 3.

Each 200 μL reaction was carried out in 96-well deep-well format (2 mL volume) with 10% (v/v) of clarified lysate, 20 g/L Compound 1, 40% (v/v) isopropanol, 40 mM TEoA buffer pH 7.5, 1 g/L NADP⁺. The plates were sealed and agitated at 30 °C at 600 rpm in Infors shaker for 24 hours.

Reactions were quenched by the addition of 800 μL acetonitrile into each well of the reactions plate, sealed and shaken for 15 minutes. The plates were then centrifuged at 4000 rpm for 10 minutes, 200 μL of the supernatant transferred to analytical plates and subjected to achiral HPLC analysis using method described in Example 5. Percent conversion and percent diastereomeric excess (% de) were calculated based on peak areas as described above. For all reported values, the fold improvement over SEQ ID NO: 54 was calculated. The results are provided in Table 5.

TABLE 5
Engineered variants relative to SEQ ID NO: 54 (Round 2)

| | SEQ | Amino Acid | FIOP | FIOP | FIOP |
|---|---|---|---|---|---|
| SEQ | ID NO: | Differences | % | % de | desired |
| ID NO: | (amino | relative to SEQ | con- | of desired | |
| (nucleotide) | acid) | ID NO: 54 | version | product | product |
| 55 | 56 | A94W | ++ | +++ | ++ |
| 57 | 58 | A94W/A202G | ++ | +++ | +++ |
| 59 | 60 | S145D | +++ | ++ | +++ |
| 61 | 62 | V95I | + | + | + |
| 63 | 64 | L17T | ++ | + | ++ |
| 65 | 66 | K192H | ++ | ++ | ++ |
| 67 | 68 | P220T | ++ | + | ++ |
| 69 | 70 | M205L | + | + | ++ |
| 71 | 72 | K192R | ++ | ++ | ++ |
| 73 | 74 | D198P | ++ | ++ | +++ |
| 75 | 76 | P194L | ++++ | ++ | ++++ |
| 77 | 78 | P194D | ++++ | ++ | ++++ |
| 79 | 80 | P194T | ++++ | ++ | +++ |
| 81 | 82 | P194E | ++++ | ++ | ++++ |
| 83 | 84 | D198V | ++ | ++ | +++ |
| 85 | 86 | P194W | +++ | +++ | +++ |
| 87 | 88 | K97I | + | ++ | + |
| 89 | 90 | T193R | ++ | +++ | +++ |
| 91 | 92 | G18S | +++ | +++ | +++ |
| 93 | 94 | S98G | + | + | ++ |
| 95 | 96 | K97M | + | + | + |
| 97 | 98 | A202L | ++ | ++ | ++ |
| 99 | 100 | L17R | ++ | ++ | ++ |
| 101 | 102 | V95M | ++ | + | ++ |
| 103 | 104 | V95P | ++ | ++ | ++ |
| 105 | 106 | M205Y | ++ | ++ | ++ |
| 107 | 108 | Q208A | ++ | + | ++ |
| 109 | 110 | L17G | + | ++ | ++ |
| 111 | 112 | P151S | + | + | + |
| 113 | 114 | P220V | + | + | + |
| 115 | 116 | D198I | ++ | ++ | +++ |
| 117 | 118 | T193G | + | ++ | ++ |
| 119 | 120 | T193Q | + | ++ | ++ |
| 121 | 122 | E106W | + | + | + |
| 123 | 124 | E200G | + | + | + |
| 125 | 126 | K192D | ++ | + | ++ |
| 127 | 128 | G18R | ++ | ++ | ++ |
| 129 | 130 | K192M | + | + | ++ |
| 131 | 132 | I223V | ++ | + | ++ |
| 133 | 134 | E106L | ++ | + | ++ |
| 135 | 136 | K211V | + | + | ++ |
| 137 | 138 | T152I | + | ++ | ++ |
| 139 | 140 | P194V | ++ | ++ | +++ |
| 141 | 142 | E106V | ++ | + | ++ |
| 143 | 144 | A155G | + | + | + |
| 145 | 146 | K97R | + | + | + |
| 147 | 148 | V196P | + | +++ | ++ |
| 149 | 150 | Q208T | ++ | + | ++ |
| 151 | 152 | V196G | ++++ | ++ | ++++ |
| 153 | 154 | E219L | + | + | + |
| 155 | 156 | I93K | + | + | + |
| 157 | 158 | A94V | ++ | + | +++ |
| 159 | 160 | V196S | + | ++ | ++ |
| 161 | 162 | G201W | ++ | + | ++ |
| 163 | 164 | Q208V | ++ | + | ++ |
| 165 | 166 | L17A | ++ | + | ++ |
| 167 | 168 | K192W | ++ | ++ | +++ |
| 169 | 170 | E106I | ++ | + | ++ |
| 171 | 172 | V113I/E200W | ++ | + | ++ |
| 173 | 174 | M205F | ++ | ++ | ++ |
| 175 | 176 | P194A | +++ | ++ | +++ |
| 177 | 178 | I223C | + | + | ++ |
| 179 | 180 | D198Y | +++ | ++ | +++ |
| 181 | 182 | T152Q/M205W/ | ++ | ++ | +++ |
| | | K211L | | | |
| 183 | 184 | M205W/K211L | ++ | ++ | +++ |
| 185 | 186 | G7S/M205L | + | + | + |

Fold improvement over positive control (FIOP) % conversion-Levels of increased activity were determined relative to the reference polypeptide of SEQ ID NO: 54 and defined as follows: “+” 1.00 to 1.50, “++” > 1.50, “+++” > 2.50, “++++” > 3.50.
Fold improvement over positive control (FIOP) % of desired product-Levels of increased selectivity were determined relative to the reference polypeptide of SEQ ID NO: 54 and defined as follows: “+” 1.00 to 1.50, “++” > 1.50, “+++” > 2.50.
Fold improvement over positive control (FIOP) of desired product-Levels of increased activity were determined relative to the reference polypeptide of SEQ ID NO: 54 and defined as follows: “+” 1.00 to 1.50, “++” > 1.50, “+++” > 2.50, “++++” > 5.00.

The best performing variants were prepared as shake flask powders and evaluated under two conditions: 20 g/L Compound 1 with or 50 g/L Compound 1 with 40% isopropanol (v/v). Reactions were prepared in 200 μL volume and both conditions contained 1 g/L NADP⁺ and 0.1 mM KPi buffer pH 7.5. 2-fold serial dilutions of enzyme shake flask powder with highest final concentration of 20 g/L were added to the reaction mixtures. Reactions were incubated at room temperature for 24 hours with shaking at 230 rpm, then quenched with acetonitrile and centrifuged at 4000 rpm for 10 minutes. 100 μL of the supernatant was transferred into Greiner microtiter plates before being subjected to HPLC analysis.

### Example 10: KRED Improvements Over SEQ ID NO: 152 for Diastereoselective Production of Compound (5s)-2 (Round 3)

SEQ ID NO: 152 (nucleic acid sequence SEQ ID NO: 151) was selected as the parent enzyme for the third round of directed evolution. Libraries of engineered genes were produced using well-established techniques (e.g., recombination of mutations identified as potentially beneficial in previous round of evolution). The enzymes encoded by each gene were produced in HTP as described in Example 2, and the clarified lysates were generated as described in Example 3.

Each 100 μL reaction was carried out in 96-well deep-well format (2 mL volume) with 10% (v/v) of clarified lysate, 150 g/L Compound 1, 40% (v/v) isopropanol, 40 mM NaPi buffer pH 7.5, 0.1 g/L NADP⁺. The plates were sealed and agitated at 40 °C at 600 rpm in Infors shaker for 24 hours.

Reactions were quenched by the addition of 900 μL acetonitrile into each well of the reactions plate, sealed and shaken for 15 minutes. The plates were then centrifuged at 4000 rpm for 10 minutes, and 70 μL of the supernatant was transferred to analytical plates, which contained 140 μL acetonitrile and was subjected to achiral HPLC analysis using the method described in Example 5. Percent conversion and percent diastereomeric excess (de) were calculated based on peak areas as described above. For the % conversion, the fold improvement over SEQ ID NO: 152 was calculated. The results are provided in Table 6.

TABLE 6
Engineered variants relative to SEQ ID NO: 152 (Round 3)

| | SEQ | Amino Acid | FIOP | % |
|---|---|---|---|---|
| SEQ | ID NO: | Differences | % | de of |
| ID NO: | (amino | relative to SEQ | con- | desired |
| (nucleotide) | acid) | ID NO: 152 | version | product |
| 187 | 188 | H40R/M205Y | + | +++ |
| 189 | 190 | S145E | + | + |
| 191 | 192 | H40R/S145E/M205L | ++++ | + |
| 19 | 194 | A94I/M205W | + | +++ |
| 195 | 196 | H40K/S145E/P194W/ | + | +++ |
| | | G196A/M205Y | | |
| 197 | 198 | A94I/M205L | +++ | + |
| 199 | 200 | S145E/M205Y | ++ | +++ |
| 201 | 202 | S145E/M205L | + | + |
| 203 | 204 | H40R/M205L | + | + |
| 205 | 206 | H40R/S145E/ | ++ | +++ |
| | | P194W/G196A/ | | |
| | | D198P/M205W | | |
| 207 | 208 | H40R/S145E | ++++ | + |
| 209 | 210 | H40R/S145D | + | +++ |
| 211 | 212 | H40R/M205W | ++ | +++ |
| 213 | 214 | H40R/S145F/P194L/ | ++ | +++ |
| | | G196A/M205Y | | |
| 215 | 216 | H40R/S145D/P194W/ | + | +++ |
| | | G196A/M205Y | | |
| 217 | 218 | S145E/M205W | ++ | +++ |
| 219 | 220 | A94I/S145D | ++++ | + |
| 221 | 222 | H40K/P194S/D198P/ | + | +++ |
| | | M205Y | | |
| 223 | 224 | H40K/S145E/M205Y | ++ | +++ |
| 225 | 226 | V56S | ++ | + |
| 227 | 228 | L21F | + | + |
| 229 | 230 | D101C | + | + |
| 231 | 232 | E100K | + | + |
| 233 | 234 | D173M | ++ | + |
| 235 | 236 | D101F | + | + |
| 237 | 238 | G7R | + | ++ |
| 239 | 240 | G7K | + | + |
| 241 | 242 | K46R | + | + |
| 243 | 244 | D197M | + | + |
| 245 | 246 | T2F | + | + |
| 247 | 248 | E45Q | + | + |
| 249 | 250 | D173W | ++ | + |
| 251 | 252 | G7C | + | + |
| 253 | 254 | D101Q | + | + |
| 255 | 256 | L17S | ++++ | + |
| 257 | 258 | V43R | + | + |
| 259 | 260 | V56D | + | ++ |
| 26 | 262 | L17M | + | + |
| 263 | 264 | V56T | ++ | + |
| 265 | 266 | T54R | + | ++ |
| 267 | 268 | E45L | + | ++ |
| 269 | 270 | D101R | ++ | ++ |
| 271 | 272 | L21S | + | ++ |
| 273 | 274 | E100A | + | ++ |
| 275 | 276 | D101N | ++ | + |
| 277 | 278 | D173V | + | ++ |
| 279 | 280 | D25T | + | + |
| 281 | 282 | G7T | + | + |
| 283 | 284 | E45R | + | + |
| 285 | 286 | K72G | + | + |
| 287 | 288 | T103E | + | + |
| 289 | 290 | D101S | + | + |
| 291 | 292 | T103W | ++ | + |
| 293 | 294 | D101T | ++ | + |
| 295 | 296 | K72T | + | + |
| 297 | 298 | D101G | + | + |
| 299 | 300 | T76K | ++ | + |
| 301 | 302 | D101V | + | + |
| 303 | 304 | R108F | + | + |
| 305 | 306 | D101L | ++++ | + |
| 307 | 308 | D101A | + | + |
| 309 | 310 | D197R | ++ | + |
| 311 | 312 | D178S | + | + |
| 313 | 314 | G135A | + | ++ |
| 315 | 316 | K177R | + | ++ |
| 317 | 318 | L176K | + | + |
| 319 | 320 | D173A | ++ | ++ |
| 321 | 322 | L17Q | ++ | ++ |
| 323 | 324 | E100R | + | ++ |
| 325 | 326 | D173L | ++ | ++ |
| 327 | 328 | H40R/T152L | ++ | ++ |
| 329 | 330 | L17M/H40K | + | +++ |
| 331 | 332 | H40R | ++ | + |
| 333 | 334 | T152L/P194T | ++ | +++ |
| 335 | 336 | L17R/H40K/ | +++ | + |
| | | K192W/P194T | | |
| 337 | 338 | L17R/P194E | +++ | + |
| 339 | 340 | L17R/H40K/M205F | + | +++ |
| 341 | 342 | L17R/T152L | ++++ | + |
| 343 | 344 | L17R/H40K/P194E | +++ | + |
| 345 | 346 | L17R | +++ | + |
| 347 | 348 | L17R/G18R/T152L | + | +++ |
| 349 | 350 | H40R/T152L/P194E | + | +++ |
| 351 | 352 | L17R/H40R/T152L/ | +++ | +++ |
| | | P194E | | |
| 353 | 354 | L17M/H40R/T152L | + | +++ |
| 355 | 356 | L17R/T152L/P194E | +++ | ++ |
| 357 | 358 | L17R/H40K/T152K | +++ | + |
| 359 | 360 | L17R/P194T | +++ | + |
| 361 | 362 | L17M/T152L | + | ++ |
| 363 | 364 | L17R/H40R/T152L | +++ | +++ |
| 365 | 366 | L17M/H40K/T152L | + | +++ |
| 367 | 368 | T152L | + | ++ |
| 369 | 370 | L17M/H40R | ++ | + |
| 371 | 372 | H40K/T152L/P194T | ++ | +++ |
| 373 | 374 | L17R/P194T/M205F | ++ | ++ |
| 375 | 376 | L17R/T152L/ | ++++ | + |
| | | K192W/P194T | | |
| 377 | 378 | L17R/G18R/H40R | + | ++ |
| 379 | 380 | L17R/T152L/ | + | ++ |
| | | P194E/L195M | | |
| 381 | 382 | L17R/M205F | + | +++ |
| 383 | 384 | L17R/H40K/ | ++++ | ++ |
| | | T152L/P194T | | |
| 385 | 386 | L17R/H40R/M205F | + | +++ |
| 387 | 388 | L17R/T152L/P194T | ++++ | ++ |
| 389 | 390 | L17R/S98C/ | ++++ | ++ |
| | | T152L/P194T | | |

Fold improvement over positive control (FIOP) % conversion-Levels of increased activity were determined relative to the reference polypeptide of SEQ ID NO: 152 and defined as follows: “+” 1.00 to 1.75, “++” > 1.75, “+++” > 2.25, “++++” > 3.00.
% of desired product-Levels of selectivity were determined as follows: “+” 90.0 to 97.5, “++” > 97.5, “+++” > 99.5.

### Example 11: KRED Improvements Over SEQ ID NO: 256 for Diastereoselective Production of Compound (5s)-2 (Round 4)

After three rounds of directed evolution, KRED enzymes were identified with an increased selectivity and an activity (% conversion) that was greater than 95% for the (5s)-2 product (Table 8). However, a fourth round of directed evolution was performed to further improve the diastereoselective production of the (5s)-2 product by improving the activity of the enzyme and reducing the concentration of the enzyme to achieve the same level of conversion and selectivity.

SEQ ID NO: 256 (nucleic acid sequence SEQ ID NO: 255) was selected as the parent enzyme for the fourth round of directed evolution. Libraries of engineered genes were produced using well-established techniques (e.g., recombination of mutations identified as potentially beneficial in previous round of evolution). The enzymes encoded by each gene were produced in HTP as described in Example 2, and the clarified lysates were generated as described in Example 3.

Each 200 μL reaction was carried out in 96-well deep-well format (2 mL volume) with 2.5% (v/v) of clarified lysate, 150 g/L Compound 1, 40% (v/v) isopropanol, 40 mM NaPi buffer pH 7.5, 0.05 g/L NADP+. The plates were sealed and agitated at 30 °C at 600 rpm in Infors shaker for 24 hours.

Reactions were quenched by the addition of 800 μL acetonitrile into each well of the reactions plate, sealed and shaken for 15 minutes. The plates were then centrifuged at 4000 rpm for 10 minutes, and 200 μL of the supernatant was transferred to analytical plates and subjected to achiral HPLC analysis using the method described in Example 5. Percent conversion and percent diastereomeric excess (% de) of desired product were calculated based on peak areas as described above. For the % conversion, the fold improvement over SEQ ID NO: 256 was calculated. The results are provided in Table 7.

TABLE 7
Engineered variants relative to SEQ ID NO: 256 (Round 4)

| | SEQ | Amino Acid | FIOP | % de |
|---|---|---|---|---|
| SEQ ID | ID NO: | Differences | % | of |
| NO: | (amino | relative to | con- | desired |
| (nucleotide) | acid) | SEQ ID NO: 256 | version | product |
| 391 | 392 | S17R/H40R/G196A | +++ | + |
| 393 | 394 | H40R | +++ | + |
| 395 | 396 | S17R/H40R/D173A | +++ | + |
| 397 | 398 | A941/A202G | ++ | + |
| 399 | 400 | H40R/E45R/V56S/D101G | + | + |
| 401 | 402 | S17R/S145E | + | + |
| 403 | 404 | S145E/D173M | ++ | + |
| 405 | 406 | S17R/H40R/E45R/A94I/ | +++ | + |
| | | D101A/S145E | | |
| 407 | 408 | H40R/V43R/A94I/D173M | +++ | + |
| 409 | 410 | S17R/S145E/D173M | + | + |
| 411 | 412 | S17R/E45R/A94I/S145E | +++ | + |
| 413 | 414 | A94I/D101R | ++ | + |
| 415 | 416 | H40R/V43R/E45R/ | ++ | + |
| | | A94I/D173M | | |
| 417 | 418 | A94I/D101G/D173M | ++ | + |
| 419 | 420 | S17R/D101G/S145E/ | ++ | +++ |
| | | D173V/A202G | | |
| 421 | 422 | H40R/V56S | + | +++ |
| 423 | 424 | S17R/E45R/S145E/ | + | +++ |
| | | D173W/A202G | | |
| 425 | 426 | A94I/S145E/A202G | ++++ | ++ |
| 427 | 428 | V43R/A202G | + | +++ |
| 429 | 430 | S145E/D173W/A202G | +++ | ++ |
| 431 | 432 | S17R/A94I | + | + |
| 433 | 434 | S17R/S145E/A202G | ++ | + |
| 435 | 436 | H40R/D101A/A202G | + | +++ |
| 437 | 438 | H40R/V56S/A202G | + | +++ |
| 439 | 440 | A94I | +++ | + |
| 441 | 442 | S17R/H40R/V43R/E45R/ | ++ | ++ |
| | | A94I/D173M/A202G | | |
| 443 | 444 | H40R/V43R/V56S | + | ++ |
| 445 | 446 | S145E/D173V | ++ | + |
| 447 | 448 | D173R/A202G | + | ++ |
| 449 | 450 | H40R/V43R/E45H/S145E | ++ | + |
| 451 | 452 | D101R/A202G | + | ++ |
| 453 | 454 | S17R/E45R/A94I/S145E/ | ++ | + |
| | | D173M | | |
| 455 | 456 | H40R/V43R/E45R/S145E/ | + | + |
| | | D173V | | |
| 457 | 458 | S17R/V56S/A94I/D101G/ | ++ | + |
| | | A202G | | |
| 459 | 460 | S17R/A94I/A202G | ++ | + |
| 461 | 462 | H40R/E45R/D101G/ | + | +++ |
| | | A202G | | |
| 463 | 464 | S17R/H40R/V43R/V56S/ | ++++ | + |
| | | A94I/S145E/D173M/ | | |
| | | A202G | | |
| 465 | 466 | S17R/V56S/A941/A202G | ++ | + |
| 467 | 468 | H40R/D101G/S145E/ | +++ | + |
| | | A170S | | |
| 469 | 470 | V43R/E45R/A941/A202G | +++ | + |
| 471 | 472 | H40R/V43R/E45R/V56S/ | + | + |
| | | A202G | | |
| 473 | 474 | H40R/A202G | + | +++ |
| 475 | 476 | H40R/V43R/V56S/A202G | + | +++ |
| 477 | 478 | V56S/A94I/D101R/A202G | + | + |
| 479 | 480 | H40R/A94I/A202G | +++ | + |
| 481 | 482 | S145E/T152L | +++ | + |
| 483 | 484 | E100A | + | + |
| 485 | 486 | E100K/S145E/M205W | ++ | + |
| 487 | 488 | D101Q/S145E | ++ | + |
| 489 | 490 | S145E | +++ | +++ |

Fold improvement over positive control (FIOP) % conversion-Levels of increased activity were determined relative to the reference polypeptide of SEQ ID NO: 256 and defined as follows: “+” 1.00 to 1.75, “++” > 1.75, “+++” > 2.25, “++++” > 3.00.
% of desired product-Levels of selectivity were determined as follows: “+” 90.0 to 97.5, “++” > 97.5, “+++” > 99.5.

After the fourth round of evolution, KRED enzymes were identified with an increased selectivity and an activity (% conversion) that was greater than 95% for the (5s)-2 product (Table 8).

TABLE 8
Directed evolution improvements

| | Selectivity (% de) | Activity (% Conv) |
|---|---|---|
| Target | >85% | >85% |
| Round #1 | 17% | 96% |
| Round #2 | 20% | >90% |
| Round #3 | >95% | >95% |
| Round #4 | >96% | >99% |

### Example 12: Evaluation of KRED Activity and Selectivity

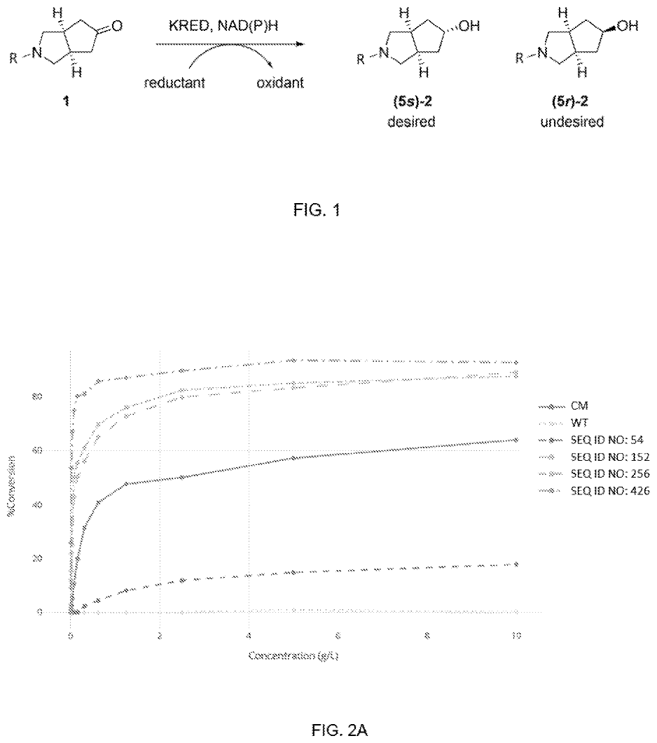

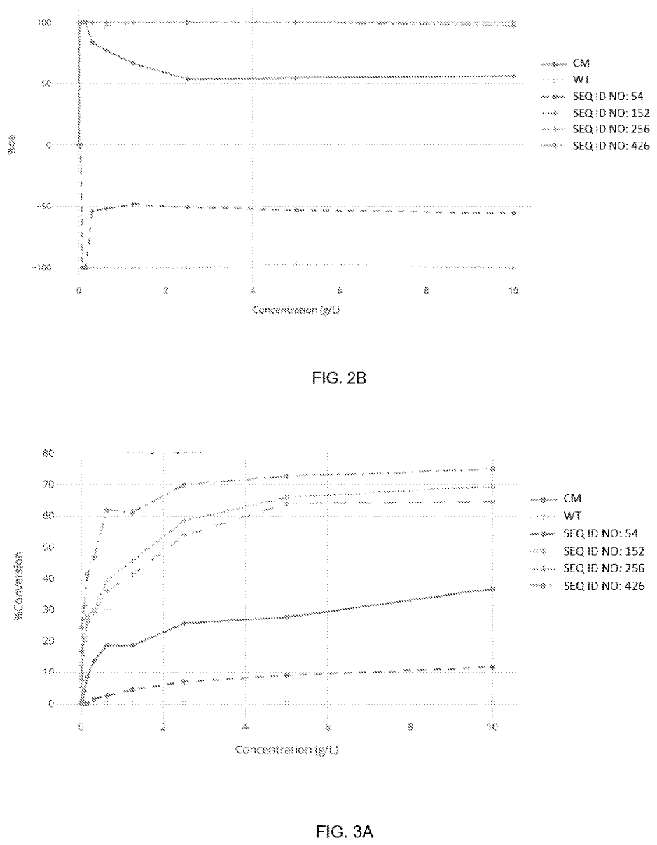

The activity and selectivity of engineered KRED enzymes SEQ ID NO: 152, SEQ ID NO: 256, and SEQ ID NO: 426 were evaluated against a commercially available KRED enzyme (CM; Johnson Matthey (ADH-152)), parental KRED enzyme (SEQ ID NO: 54), and wild-type (WT; SEQ ID NO: 492) by performing an enzyme dose curve in a 96-well plate format under several testing conditions (FIGS. 2-5).

Two testing conditions were used for measuring activity (% conversion) and selectivity (% de). Parameters were selected to allow the enzymes to exhibit the highest performance. The assessment of the enzymes was evaluated on the highest achieved conversion and selectivity independent from the evaluated condition. Specific evaluation parameters are provided in Table 9.

TABLE 9
Evaluation Parameters

| Parameters | Condition 1 | Condition 2 |
|---|---|---|
| Substrate | 50 g/L; 91 g/L | 50 g/L; 100 g/L; |
| concentration | | 150 g/L |
| Enzyme | 10 g/L highest | 10 g/L highest |
| concentration | KRED enzyme | KRED enzyme |
| | concentration, | concentration, |
| | serial dilution | serial dilution |
| | factor of 2 | factor of 2 |
| Cofactor | 2% w/w | 0.1% w/w; 0.05% |
| concentration | | w/w; 0.03% |
| (NADP+) | | w/w |
| Buffer | 100 mM Sodium | 40 mM Sodium |
| | Phosphate pH7.5 | Phosphate pH7.5; |
| | | 4 mM MgSO₄ |
| Reaction temperature | 33 °C | 40 °C |
| Reaction time | 24 hrs | 24 hrs |
| Cofactor recycling | GDH 1 wt %/Glucose | none |
| Conversion | ~80% | >95% |
| pH adjustment | NaOH | none |
| Co-solvent | DMSO 16% | iPrOH 40% |
| Work-up | Extraction | Concentration and |
| | | precipitation |

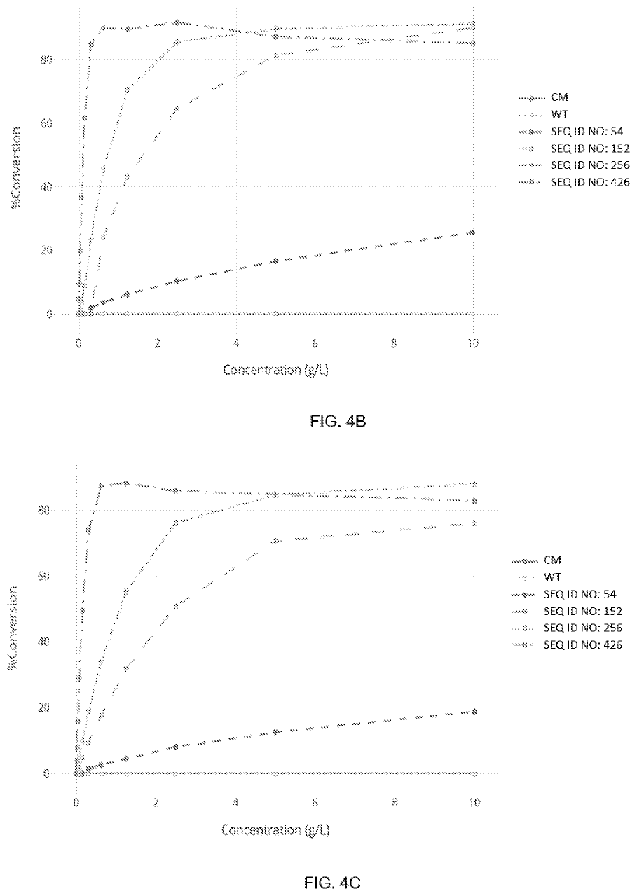

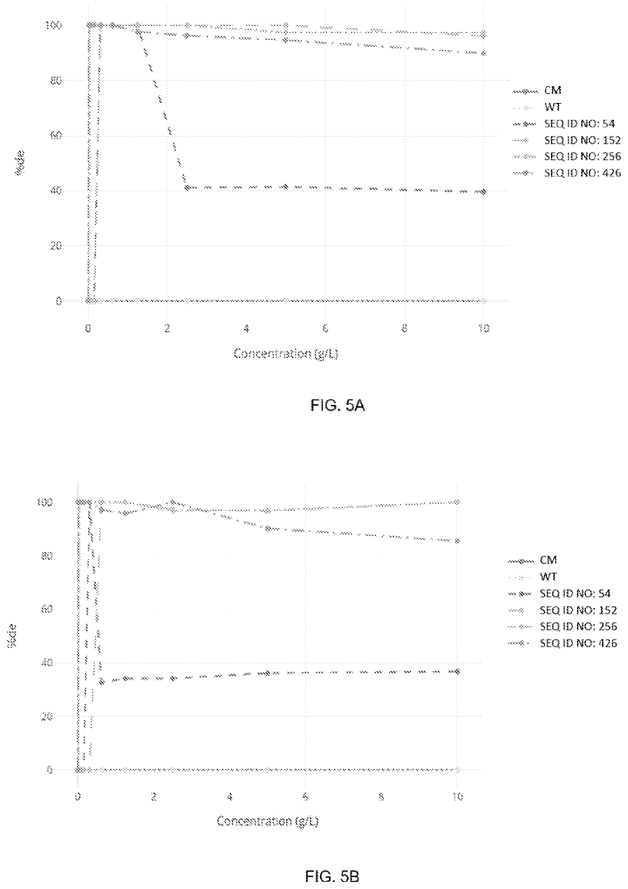

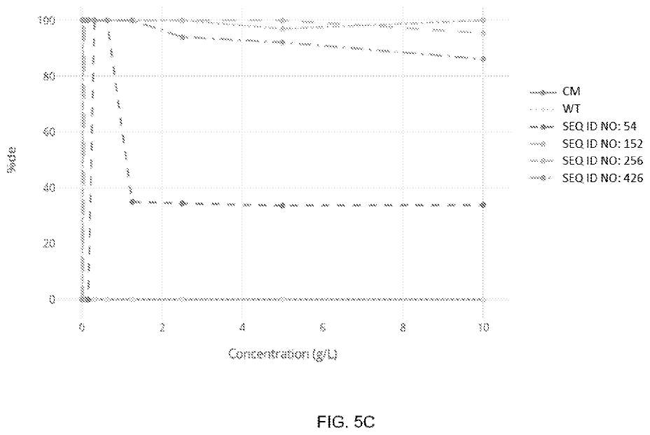

Under Conditions 1 and 2, engineered KRED enzymes SEQ ID NO: 152, SEQ ID NO: 256, and SEQ ID NO: 426 demonstrated better activity (FIGS. 2A, 3A, 4A) and selectivity (FIGS. 2B, 3B, 5A) as compared to the commercial enzyme, the parental KRED, and wild-type. Exemplary enzyme SEQ ID NO: 426 exhibited the highest activity (FIGS. 2A, 3A, 4A) and selectivity (FIGS. 2B, 3B, 5A). The improved performance of SEQ ID NO: 426 was also observed under the screening conditions at increasing substrate concentrations (FIGS. 4A-4C, 5A-5C). Specifically, exemplary enzyme SEQ ID NO: 426 achieved at 50 g/L [Substrate] and 5% w/w enzyme load, a 97% conversion and 99.6% de; and at 100 g/L [Substrate] and 1% w/w enzyme load, a 93% conversion and 98.8% de. The commercial enzyme was not active under Condition 2 without the cofactor recycling enzyme GDH/Glucose (FIGS. 4A, 5A) indicating that it is a prerequisite for activity. The WT control showed no activity under either condition.

### Example 13: KRED Immobilization

### Preparation of Immobilization Buffer

To prepare a phosphate buffer solution (0.05 M pH=7.5) containing magnesium sulfate (0.002 M), Na₂HPO₄ 12 H₂O (230.18 g), NaH₂PO₄ 2 H₂O (16.58 g) and MgSO₄ (3.61 g) were dissolved in water (15 L). 12.2 L of this solution was used in the immobilization process. The buffer concentration may range from 0.01-0.10 M. Sodium phosphate salts may also be substituted by potassium phosphate salts.

### Preparation of Glutaraldehyde 0.5% Solution in Phosphate Buffer

To prepare glutaraldehyde 0.5% solution in phosphate buffer, glutaraldehyde 25% in water (24.5 mL) was mixed with 0.05 M pH=7.5 phosphate buffer (1200 mL). A total of 1160 mL of this solution was used in the immobilization process. The concentration of glutaraldehyde may range: 0.2-2%. As alternative to glutaraldehyde, other bifunctional linkers may be used to bind the enzyme residues to the resin.

### Preactivation of the Enzyme Carrier

The immobilization carrier was selected after several screenings of amino- and epoxy-functionalized resins from commercial suppliers, with different particle size, pore diameter and hydrophobicity.

Amino-functionalized resin Lifetech ECR8304F (325 g) was washed three times with immobilization buffer (580 mL) and incubated for two hours with 0.5% glutaraldehyde solution (1160 mL) at room temperature. The preactivated resin was then washed four times with immobilization buffer (1160 mL).

### KRED Immobilization

To the preactivated resin, a solution of KRED (16 g) in immobilization buffer (1160 mL) was added and the mixture was incubated at room temperature for 18 hours. The immobilized enzyme was washed with immobilization buffer (1160 mL), 0.5 M NaCl solution (2×1160 mL) and immobilization buffer (2×1160 mL). This procedure afforded ca. 400 g of wet immobilized KRED, with an immobilization yield of 68-69% and a 66% recovered enzymatic activity.

The enzyme/resin ratio may range from 10-100 mg enzyme/g resin. The enzyme/resin ratio (ca. 50 mg enzyme/g resin) was selected to maximize the recovery of enzymatic activity after the immobilization process and the specific activity of the final immobilisate. This could be further optimized and lower enzyme/resin ratios may possibly lead to higher activity recovery, while having low impact on the specific activity of the final immobilized KRED. A broader temperature and time range could be included. The amount of washing solutions and number of rinses was adapted from the resin provider suggested procedure, but can be further optimized. The addition of cofactor NADP during incubation was evaluated in the DoE as a measure for increasing enzymatic activity retention and may be used as an optional additive.

### Biocatalytic Reaction

To prepare a phosphate buffer solution (0.1 M pH=7.5) containing magnesium sulfate, Na₂HPO₄ 12 H₂O (152 g), NaH₂PO₄ 2 H₂O (11.7 g) and Magnesium sulfate (6.3 g) were dissolved in water (5 L, 2 kg of this are used in the reaction). The pH was adjusted to 7.4-7.6 using NaOH 3N or Phosphoric acid 29%. A buffer concentration range of 0.01-1.00 M may be used.

The biocatalytic reaction procedure was run three times, using recovered immobilized enzyme in the 2^{nd} and 3^{rd} batch. Substrate (1.00 kg) was dissolved in iPrOH (1.95 kg). Water (2.5 kg) and phosphate buffer (1.9 kg) were added and the pH was controlled and adjusted if necessary (pH range=7.5-8.3). NADPNa (2 g) was added as a solution in phosphate buffer (100 g). Immobilized KRED (380 g) was added as a slurry in a 1:1 (v/v) mixture of water and isopropanol (875 mL: 875 mL). The enzyme container was then rinsed with a 1:1 (v/v) mixture of water and isopropanol (625 mL: 625 mL), into the reaction vessel. The pH was controlled and adjusted if necessary (target pH range=7.5-8.3). The mixture was warmed to IT=35 °C and stirred for 20 h. An azeotrope mixture of acetone, isopropanol and water was distilled off (2 L), and was replaced by a 1:1 (w/w) mixture of water and isopropanol (1 kg: 1 kg). The mixture was stirred at IT=35 °C for further 10 h. Temperature ranges, reaction times, and NADP loading may be further optimized.

### Workup/Isolation:

The reaction mixture was filtered through a Pall K900 depth filter and the immobilized enzyme was washed with a 1:1 (v/v) mixture of water and isopropanol (3×1L:1L). The filtrates were combined and isopropanol (10 L) was evaporated therefrom at JT=50 °C under reduced pressure, while part of the volume distilled out was replaced by the addition of water (5 kg). The precipitated product was stirred for 18 h at 25 °C.

The product was isolated by filtration. The filter cake was washed with water (2 kg). The wet product was dried at 60 °C and full vacuum to afford product as a white solid (843-895 g, 84-89% yield, range from the three batches)

Clear filtration over Cellflock was avoided by using the immobilized enzyme. This resulted in a significantly reduced filtration time: from 83 minutes in a 2.5 kg-batch using free enzyme, to 3-9 minutes in the three 1-kg batches using immobilized KRED. All three batches met the specifications, affording yield and purity values comparable to those of the non-immobilized enzyme process. By using the enzyme in a total of 3 batches, the amount of KRED was reduced from 1% (w/w) to 0.5% (w/w). Distillation and amount of water added depended on the starting concentration, if modified.

### Enzyme Storage Between Batches

The immobilized enzyme was stored as a slurry in a 1:1 (v/v) mixture of water and isopropanol (1L:1L) at 2-8 °C and was used in a total of three batches, with a maximum of 5 weeks storage time between two batches. The amount of 1:1 water:isopropanol solution added to the reaction vessel in the second and third batch was adapted based on the volume of enzyme slurry stored.

For the storage of the immobilized enzyme between batches, a screening of different stabilizing solutions was performed over a period of one month. Finally, isopropanol/water 1:1 was selected, as the immobilized enzyme retained 100% of its initial activity after 1 month of storage in this solution. By using isopropanol/water 1:1, no rinsing of the immobilized KRED enzyme was required between batches. Moreover, use of isopropanol/water 1:1 prevented the growth of microorganisms during storage. SEM images (not shown) of immobilized KRED beads stored in aqueous buffer for several months showed the growth of bacteria on the surface, whereas no colonies were observed on the immobilized KRED beads stored in isopropanol/water 1:1.

### Immobilization Parameters

The following parameters were investigated.

### Enzyme Carriers:

Amino-functionalized, different pore sizes, different providers: Relizyme EA113, Purolite Lifetech ECR8309F & ECR8304F
Epoxy-functionalized, different hydrophobicity and pore size: Purolite Lifetech ECR8204F & ECR8285
   Upon selection of Lifetech ECR8304, the following parameters were studied:
Enzyme/carrier ratio (mg enzyme/carrier): 10-100 mg/g
Enzyme solution concentration: 2.5-50 mg/mL
Glutaraldehyde % used as linker between enzyme and the amino-functionalized carrier: 0.2-2%
NADP sodium salt as additive to increase enzymatic activity retention: 0-3 mg/mL in enzyme solution

### EQUIVALENTS

The disclosures of each and every patent, patent application, and publication cited herein are hereby incorporated herein by reference in their entirety. While this invention has been disclosed with reference to certain embodiments, it is apparent that further embodiments and variations of this invention may be devised by others skilled in the art without departing from the true spirit and scope of the invention. The appended claims are intended to be construed to include all such embodiments and equivalent variations.

What is claimed is:

1. An engineered ketoreductase polypeptide, wherein the polypeptide is selected from any of the following:
a) a polypeptide comprising an amino acid sequence having at least about 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% sequence identity to an amino acid sequence selected from the group consisting of SEQ ID NOs: 392, 394, 396, 406, 408, 420, 422, 424, 426, 428, 430, 436, 438, 462, 464, 468, 470, 474, 476, 480, 482, and 490;
b) a polypeptide comprising an amino acid sequence having (1) at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 54, 152, 256, or 492; and (2) comprising one or more amino acid differences relative to said amino acid sequence selected from:
i) X17S, X17T, or X17G;
ii) X18S or X18R;
iii) X56S, X56T, or X56D;
iv) X94I;
v) X106I, X106V, X106L, or X106W;
vi) X173A, X173W, X173M, or X173K;
vii) X194A, X194V, X194W, or X194T;
viii) X196G; and
ix) X198V, X198I, X198Y, or X198P,
optionally comprising one or more additional amino acid residue differences relative to said amino acid sequence; or
c) a polypeptide comprising an amino acid sequence selected from Table 4 (SEQ ID NOs: 4-52), Table 5 (SEQ ID NOs: 56-186), Table 6 (SEQ ID NOs: 188-390) or Table 7 (SEQ ID NOs: 392-490).

2. The engineered polypeptide according to claim 1, wherein the polypeptide is capable of selectively reducing tert-butyl rel-(3aR,6aS)-5-oxohexahydrocyclopenta[c]pyrrole-2(11)-carboxylate (Substrate) to tert-butyl rel-(3aR,5s,6aS)-5-hydroxyhexahydrocyclopenta[c]pyrrole-2(1H)-carboxylate (Product).

3. An engineered ketoreductase polypeptide capable of selectively reducing tert-butyl rel-(3aR,6aS)-5-oxohexahydrocyclopenta[c]pyrrole-2(1H)-carboxylate (Substrate) to tert-butyl rel-(3aR,5s,6aS)-5-hydroxyhexahydrocyclopenta[c]pyrrole-2(1H)-carboxylate (Product), wherein the polypeptide comprises an amino acid sequence having (i) at least about 80%, 85%, 90%, 95%, 96%, 97%, 98%, 99%, or 100% sequence identity to SEQ ID NO: 54, 152, 256, or 492 and (ii) a substitution, deletion, addition or insertion of one or more amino acid residues selected from Table 2, Table 4, Table 5, Table 6, or Table 7 relative to said amino acid sequence, optionally comprising one or more additional amino acid residue differences relative to said amino acid sequence.

4. The engineered polypeptide according to any one of claims 1 to 3, wherein the engineered polypeptide amino acid sequence comprises or more amino acid differences at positions X17, X18, X40, X56, X94, X96, X106, X145, X173, X190, X194, X196, X198, X202, and/or X206 relative to said amino acid sequence, optionally comprising one or more additional amino acid residue differences relative to said amino acid sequence.

5. The engineered polypeptide according to any one of claims 1 to 4, wherein the engineered polypeptide amino acid sequence comprises (i) a residue at position X190 that is not tyrosine; (ii) a residue at position X190 that is a non-aromatic residue; (iii) a residue at position X196 that is an aliphatic residue or a small amino acid residue; (iv) a residue at position X202 that is a small amino acid residue; and/or (v) a residue at position X206 that is not methionine, relative to said amino acid sequences, optionally comprising one or more additional amino acid residue differences relative to said amino acid sequence.

6. The engineered polypeptide according to any one of claims 1 to 5, wherein the engineered polypeptide amino acid sequence comprises one or more of the following amino acid residues: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198L, X198Y, X198D, X198P, X202A, X202G, X206Q, X206N and/or X206W relative to said amino acid sequences, optionally comprising one or more additional amino acid residue differences relative to said amino acid sequence.

7. The engineered polypeptide according to any one of claims 1 to 6, wherein the engineered polypeptide amino acid sequence comprises one or more amino acid residues selected from X94I, X96Y, X190A, X196G, X202A, X202G and/or X206W relative to said amino acid sequences, optionally comprising one or more additional amino acid residue differences relative to said amino acid sequence.

8. The engineered polypeptide according to any one of claims 1 to 7, wherein the engineered polypeptide amino acid sequence comprises one or more amino acid residues selected from X18G, X40H, X56V, X94A, X106E, X145E, X145, X173D, X194P, X198D, X202A, and X206Q relative to said amino acid sequences, optionally comprising one or more additional amino acid residue differences relative to said amino acid sequence.

9. The engineered polypeptide according to any one of claims 1 to 8, wherein the engineered polypeptide amino acid sequence comprises an amino acid sequence selected from the group consisting of: SEQ ID NOs: 392, 394, 396, 406, 408, 420, 422, 424, 426, 428, 430, 436, 438, 462, 464, 468, 470, 474, 476, 480, 482, and 490.

10. The engineered polypeptide according to any one of claims 1 to 9, wherein the engineered polypeptide is solvent stable.

11. The engineered polypeptide according to any one of claims 2 to 10, wherein the engineered polypeptide reduces Substrate to Product with a conversion rate of at least about 85%, at least about 90%, at least about 95%, at least about 96%, at least about 97%, at least about 98%, at least about 99.0%, or 100%.

12. The engineered polypeptide according to any one of claims 2 to 11, wherein the engineered polypeptide reduces Substrate to Product with a level of selectivity (% diastereomeric excess) of at least about 96.0%, at least about 96.5%, at least about 97.0%, at least about 97.5%, at least about 98.0%, at least about 98.5%, at least about 99.0%, at least about 99.5%, or 100%.

13. The engineered polypeptide according to any one of claims 2 to 12, wherein the engineered polypeptide does not require glucose dehydrogenase (GDH)/glucose cofactor recycling for reducing Substrate to Product.

14. The engineered polypeptide according to any one of claims 2 to 13, wherein the capability of reducing Substrate to Product is relative to a reference polypeptide.

15. The engineered polypeptide according to claim 14, wherein the engineered polypeptide has a reversed or increased diastereoselectivity (percent diastereomeric excess (% de), relative to the reference polypeptide for reducing Substrate to Product.

16. The engineered polypeptide according to claim 14 or 15, wherein the engineered polypeptide has a level of increased activity (e.g., conversion rate) relative to the reference polypeptide with a fold improvement over positive control (FIOP) greater than about 1.75, greater than about 2.00, greater than about 2.25, greater than about 2.50, greater than about 2.75, greater than about 3.00, greater than about 3.25, greater than about 3.50, greater than about 3.75, greater than about 4.00, greater than about 4.25, greater than about 4.50, greater than about 4.75, greater than about 5.00, greater than about 5.25, greater than about 5.50, greater than about 5.75, greater than about 6.00, greater than about 6.25, or greater than about 6.50.

17. The engineered polypeptide according to any one of claims 14 to 16, wherein the engineered polypeptide is capable of reducing Substrate to Product with a FIOP conversion rate greater than about 2.25, preferably greater than about 3.00, and a diastereoselectivity greater than about 95%, preferably greater than about 97%, or more preferably greater than about 99%, as compared to the reference polypeptide.

18. The engineered polypeptide according to any one of claims 14 to 17, wherein the reference polypeptide is a wild-type Lactobacillus kefir, Lactobacillus brevis, or Lactobacillus minor ketoreductase polypeptide.

19. The engineered polypeptide according to any one of claims 14 to 18, wherein the reference polypeptide is a wild-type Lactobacillus kefir ketoreductase polypeptide.

20. The engineered polypeptide according to any one of claims 14 to 19, wherein the reference polypeptide is a wild-type Lactobacillus kefir ketoreductase polypeptide comprising an amino acid sequence of SEQ ID NO: 492.

21. The engineered polypeptide according to any one of claims 14 to 17, wherein the reference polypeptide is an engineered ketoreductase polypeptide.

22. The engineered polypeptide according to any one of claims 14-17 or 21, wherein the reference polypeptide is an engineered ketoreductase polypeptide selected from SEQ ID NO: 54, SEQ ID NO: 152, or SEQ ID NO: 256.

23. The engineered polypeptide according to any one of claims 14-17, 21 or 22, wherein the engineered polypeptide i) requires less cofactor; ii) does not require glucose dehydrogenase (GDH)/glucose cofactor recycling; and/or iii) does not require dimethylsulfoxide (DMSO), as compared to the reference polypeptide in a reduction reaction for reducing Substrate to Product.

24. An engineered ketoreductase polypeptide capable of selectively reducing tert-butyl rel-(3aR,6aS)-5-oxohexahydrocyclopenta[c]pyrrole-2(1H)-carboxylate (Substrate) to tert-butyl rel-(3aR,5s,6aS)-5-hydroxyhexahydrocyclopenta[c]pyrrole-2(1H)-carboxylate (Product) under suitable reaction conditions at a greater stereoselectivity (% diastereomeric excess) and/or activity (% conversion) than that of SEQ ID NO: 54, SEQ ID NO: 152, and/or SEQ ID NO: 256.

25. The engineered polypeptide according to claim 24, wherein the suitable reaction conditions include one or more of the following: i) up to about 150 g/L, e.g., 50 g/L or 100 g/L, of Substrate; ii) enzyme loading less than about 5 wt %, e.g., less than about 3 wt %, e.g., less than about 1 wt %; iii) NADP+ cofactor loading less than about 2 wt %, e.g., less than about 1 wt %, e.g., less than about 0.2 wt %, e.g., 0.1%, 0.05%, or 0.03%; iv) up to 40% (v/v) of isopropanol; v) a temperature of about 15 to 75 °C, e.g., 20 to 55 °C, e.g., 20 to 45 °C, e.g., 30 °C or 40 °C; vi) a pH of 5.0 to 10.0, e.g., pH of 7.5-8.3; and vii) a reaction time of up to 30 hours, preferably 24 hours.

26. The engineered polypeptide according to claim 24 or 25, wherein the suitable reaction conditions do not require dimethylsulfoxide (DMSO).

27. The engineered polypeptide according to any one of claims 24 to 26, wherein the suitable reaction conditions do not require GDH/Glucose cofactor recycling.

28. The engineered polypeptide according to any one of claims 24 to 27, wherein the engineered polypeptide comprises an amino acid sequence that differs from the amino acid sequence of SEQ ID NO: 54, SEQ ID NO: 152, or SEQ ID NO: 256 by one or more amino acid residues selected from: Table 2, Table 4, Table 5, Table 6, or Table 7, optionally comprising one or more additional amino acid residue differences relative to said amino acid sequence.

29. The engineered polypeptide according to any one of claims 24 to 28, wherein the engineered polypeptide comprises an amino acid sequence that differs from the amino acid sequence of SEQ ID NO: 54, SEQ ID NO: 152, or SEQ ID NO: 256 in one or more amino acid residues selected from: 17, 18, 40, 56, 94, 96, 106, 145, 173, 190, 194, 196, 198, 202, and/or 206, optionally comprising one or more additional amino acid residue differences relative to said amino acid sequence.

30. The engineered polypeptide according to any one of claims 24 to 29, wherein the engineered polypeptide amino acid sequence comprises (i) a residue at position X190 that is not tyrosine; (ii) a residue at position X190 that is a non-aromatic residue; (iii) a residue at position X196 that is an aliphatic residue or a small amino acid residue; (iv) a residue at position X202 that is a small amino acid residue; and/or (v) a residue at position X206 that is not methionine, relative to the amino acid sequence of SEQ ID NO: 54, SEQ ID NO: 152, or SEQ ID NO: 256, optionally comprising one or more additional amino acid residue differences relative to said amino acid sequence.

31. The engineered polypeptide according to any one of claims 24 to 30, wherein the engineered polypeptide amino acid sequence comprises one or more amino acid residues selected from: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198I, X198Y, X198D, X198P, X202A, X202G, X206Q, X206N and/or X206W relative to the amino acid sequence of SEQ ID NO: 54, SEQ ID NO: 152, or SEQ ID NO: 256, optionally comprising one or more additional amino acid residue differences relative to said amino acid sequence.

32. The engineered polypeptide according to any one of claims 24 to 31, wherein the engineered polypeptide amino acid sequence comprises one or more amino acid residues selected from:
i) X17S, X17T, or X17G;
ii) X18S or X18R;
iii) X56S, X56T, or X56D;
iv) X94I;
iv) X106I, X106V, X106L, or X106W;
vi) X173A, X173W, X173M, or X173K;
vii) X194A, X194V, X194W, or X194T;
vii) X196G; and
viii) X198V, X198I, X198Y, or X198P;
relative to the amino acid sequence of SEQ ID NO: 54, SEQ ID NO: 152, or SEQ ID NO: 256, optionally comprising one or more additional amino acid residue differences relative to said amino acid sequence.

33. The engineered polypeptide according to any one of claims 24 to 32, wherein the engineered polypeptide amino acid sequence comprises one or more amino acid residues selected from: X94I, X96Y, X190A, X196G, X202A, X202G and/or X206W, relative to the amino acid sequence of SEQ ID NO: 54, SEQ ID NO: 152, or SEQ ID NO: 256, optionally comprising one or more additional amino acid residue differences relative to said amino acid sequence.

34. The engineered polypeptide according to any one of claims 24 to 33, wherein the engineered polypeptide amino acid sequence comprises one or more amino acid residues selected from: X18G, X40H, X56V, X94A, X106E, X145E, X145, X173D, X194P, X198D, X202A, and X206Q, relative to the amino acid sequence of SEQ ID NO: 54, SEQ ID NO: 152, or SEQ ID NO: 256, optionally comprising one or more additional amino acid residue differences relative to said amino acid sequence.

35. The engineered polypeptide according to any one of claims 24 to 34, wherein the engineered polypeptide comprises an amino acid sequence selected from:
a) an amino acid sequence selected from Table 4 (SEQ ID NOs: 4-52), Table 5 (SEQ ID NOs: 56-186), Table 6 (SEQ ID NOs: 188-390) or Table 7 (SEQ ID NOs: 392-490), preferably an amino acid sequence selected from Table 7; or
b) an amino acid sequence having at least about 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% sequence identity to an amino acid sequence selected from the group consisting of SEQ ID NOs: 392, 394, 396, 406, 408, 420, 422, 424, 426, 428, 430, 436, 438, 462, 464, 468, 470, 474, 476, 480, 482, and 490.

36. A method for the diastereoselective reduction of a bicyclic ketone substrate, the method comprising the step of contacting the bicyclic ketone substrate with a ketoreductase (KRED), under suitable reaction conditions, to obtain a bicyclic secondary alcohol product.

37. The method of claim 36, wherein the KRED is an engineered ketoreductase.

38. A method for the diastereoselective reduction of a bicyclic ketone substrate, the method comprising the step of contacting the bicyclic ketone substrate with the engineered polypeptide according to any one of claims 1 to 35, under suitable reaction conditions, to obtain a bicyclic secondary alcohol product.

39. The method according to any one of claims 36 to 38, wherein the bicyclic ketone substrate has between 6 and 12 members in total.

40. The method according to any one of claims 36 to 39, wherein the bicyclic ketone substrate is achiral.

41. The method according to any one of claims 36 to 40, wherein the bicyclic secondary alcohol product has the structure shown in formula (IB): 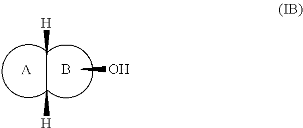
wherein:
the A ring and the B ring together represent a fused cycloalkyl ring, e.g., C₆-C₁₂ cycloalkyl, or fused heterocyclyl ring, e.g., 6-12 membered heterocyclyl, wherein the fused cycloalkyl or heterocyclyl is optionally substituted with at least one occurrence of R¹, e.g., one to four R¹,
each R¹ is independently selected from an amine protecting group, e.g., tert-butyloxycarbonyl (Boc), N-carboxybezyl (Cbz), C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, 5-20 membered heteroaryl, hydroxyl, halogen, e.g., F, C₁-C₂₀haloalkyl, e.g., —CF₃, —OR^{c}, —NR^{c}R^{c}, —(CH₂)ₙCOOR^{c}, —(CH₂)ₙ—C(═O)R^{c}, and —(CH₂)ₙ—C(═O)NR^{c}R^{c};
wherein the alkyl, alkenyl, and alkynyl are each optionally substituted by one or more R^{a}, e.g., one to six R^{a},
wherein the cycloalkyl, aryl, arylalkyl, heterocyclyl, and heteroaryl are each optionally substituted by one or more R^{b}, e.g., one to six R^{b};
each R^{a} is at each occurrence independently selected from C₃-C₁₀cycloalkyl, C₆-C₁₄aryl, C₇-C₂₀arylalkyl, 3-14 membered heterocyclyl, and 5-20 membered heteroaryl, halogen, e.g., F, haloalkyl, e.g., C₁-C₂₀haloalkyl, e.g., —CF₃, —OR^{c}, —NR^{c}R^{c}, —(CH₂)ₙCOOR^{c}, —(CH₂)ₙ—C(═O)R^{c}, and —(CH₂)ₙ—C(═O)NR^{c}R^{c};
each R^{b} is at each occurrence independently selected from halogen, C₁-C₂₀haloalkyl, e.g., —CF₃, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c}, C₁-C₂₀alkyl, C₂-C₂₀alkenyl, and C₂-C₂₀alkynyl;
each R^{c} is at each occurrence independently selected from H, C₁-C₂₀alkyl, C₂-C₂₀alkenyl, and C₂-C₂₀alkynyl, each optionally substituted by one or more R^{b}, e.g., one to six R^{b}; and
n is 0, 1, 2, 3, 4, 5 or 6.

42. The method according to any one of claims 36 to 41, wherein the bicyclic ketone substrate has the structure shown in formula (IA):

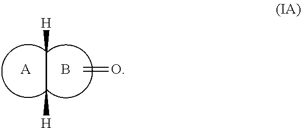

43. The method according to claim 41 or 42, wherein the A ring and the B ring together represent a fused 6-12 membered heterocyclyl comprising at least one nitrogen heteroatom.

44. The method according to claim 43, wherein the nitrogen heteroatom of the bicyclic ketone substrate is bonded to an amine protecting group, e.g., tert-butyloxycarbonyl (Boc), N-carboxybezyl (Cbz).

45. The method according to any one of claims 36 to 44, wherein the bicyclic ketone substrate is of formula (IA)-I 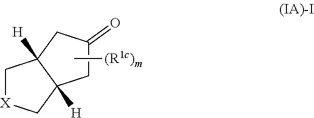
wherein:
X is selected from N—R^{1a}, CH₂ and CH—R^{1b}
R^{1a} is selected from an amine protecting group, e.g., tert-butyloxycarbonyl (Boc), N-carboxybezyl (Cbz), C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, and 5-20 membered heteroaryl;
R^{1b} is selected from C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, and 5-20 membered heteroaryl;
wherein the alkyl, alkenyl, and alkynyl of R^{1a} or R^{1b} are each optionally substituted by one to six R^{a};
wherein the cycloalkyl, aryl, arylalkyl, heterocyclyl, and heteroaryl of R^{1a} or R^{1b} are each optionally substituted by one to six R^{b};
each R^{1c} is at each occurrence independently selected from C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, and 5-20 membered heteroaryl, hydroxyl, halogen, e.g., F, C₁-C₂₀haloalkyl, e.g., —CF₃, —OR^{c}, —NR^{c}R^{c}, —(CH₂)ₙCOOR^{c}, —(CH₂)ₙ—C(═O)R^{c}, and —(CH₂)ₙ—C(═O)NR^{c}R^{c};
each R^{a} is at each occurrence independently selected from C₃-C₁₀cycloalkyl, C₆-C₁₄aryl, C₇-C₂₀arylalkyl, 3-14 membered heterocyclyl, and 5-20 membered heteroaryl, halogen, e.g., F, haloalkyl, e.g., C₁-C₂₀haloalkyl, e.g., —CF₃, —OR^{c}, —NR^{c}R^{c}, —(CH₂)ₙCOOR^{c}, —(CH₂)ₙ—C(═O)R^{c}, and —(CH₂)ₙ—C(═O)NR^{c}R^{c};
each R^{b} is at each occurrence independently selected from halogen, C₁-C₂₀haloalkyl, e.g., —CF₃, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c}, C₁-C₂₀alkyl, C₂-C₂₀alkenyl, and C₂-C₂₀alkynyl;
each R^{c} is at each occurrence independently selected from H, C₁-C₂₀alkyl, C₂-C₂₀alkenyl, and C₂-C₂₀alkynyl, each optionally substituted by one to six R^{b};
n is 0, 1, 2 or 3; and
m is 0, 1 or 2.

46. The method according to claim 45, wherein,
X is N—R^{1a};
R^{1a} is selected from C₁-C₁₀alkyl, C₃-C₁₀cycloalkyl, C₆-C₁₄aryl, C₇-C₂₀arylalkyl, and an amine protecting group, e.g., tert-butyloxycarbonyl (Boc), N-carboxybezyl (Cbz); and
m is 0.

47. The method according to claim 45 or 46, wherein X is N—R^{1a}, and R^{1a} is an amine protecting group, e.g., tert-butyloxycarbonyl (Boc), N-carboxybezyl (Cbz).

48. A method for stereoselectively reducing a compound of formula (IA)-I to (IB)-I: 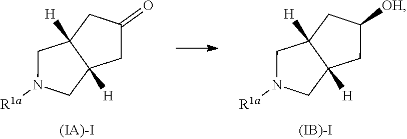
wherein R^{1a} is an amine protecting group, the method comprising the step of contacting the substrate of formula (IA)-I with a ketoreductase (KRED) under reaction conditions suitable for reducing or converting (IA)-I to (TB)-I.

49. The method of claim 48, wherein the KRED is an engineered ketoreductase.

50. The method of claim 48 or 49, wherein the KRED is an engineered polypeptide of any one of claims 1 to 35.

51. The method of any one of claims 48 to 50, wherein the amine protecting group is tert-butyloxycarbonyl (Boc) or N-carboxybenzyl (Cbz).

52. A method for stereoselectively reducing tert-butyl rel-(3aR,6aS)-5-oxohexahydrocyclopenta[c]pyrrole-2(1H)-carboxylate (substrate) to tert-butyl rel-(3aR,5s,6aS)-5-hydroxyhexahydrocyclopenta[c]pyrrole-2(1H)-carboxylate (product): 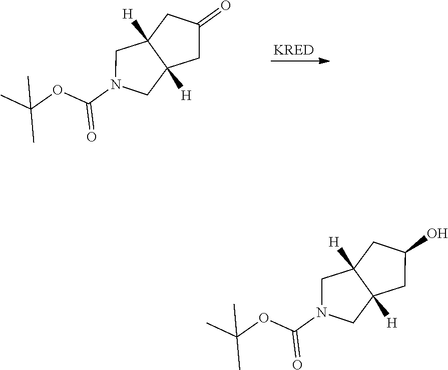 [SMILES: CC(C)(C)OC(=O)N1C[C@H]2CC(=O)C[C@H]2C1 ; CC(C)(C)OC(=O)N1C[C@H]2C[C@H](O)C[C@H]2C1]
the method comprising the step of contacting the substrate tert-butyl rel-(3aR,6aS)-5-oxohexahydrocyclopenta[c]pyrrole-2(1H)-carboxylate with a ketoreductase (KRED) under reaction conditions suitable for reducing or converting tert-butyl rel-(3aR,6aS)-5-oxohexahydrocyclopenta[c]pyrrole-2(1H)-carboxylate to tert-butyl rel-(3aR,5s,6aS)-5-hydroxyhexahydrocyclopenta[c]pyrrole-2(1H)-carboxylate.

53. The method of claim 52, wherein the KRED is an engineered KRED.

54. The method of claim 52 or 53, wherein the KRED is an engineered polypeptide of any one of claims 1 to 35.

55. The method according to any one of claims 36-54, wherein the reaction is carried out in a solvent.

56. The method according to claim 55, wherein the solvent is selected from a polar solvent, non-polar solvent and ionic liquid.

57. The method according to claim 55 or 56, wherein the solvent is selected from water, methanol, ethanol, n-propanol, isopropanol, isopropyl acetate, dimethyl sulfoxide, dimethylformamide, ethyl acetate, butyl acetate, 1-octanol, hexane, heptane, octane, methyl tert-butyl ether, toluene, 1-ethyl-4-methylimidazolium tetrafluoroborate, 1-butyl-3-methylimidazolium tetrafluoroborate, 1-butyl-3-methylimidazolium hexafluorophosphate, glycerol, ethylene glycol, propylene glycol, and polyethylene glycol.

58. The method according to any one of claims 55 to 57, wherein the solvent is isopropanol.

59. The method according to any one of claims 36 to 58, wherein the reaction is carried out in up to 40 wt % of isopropanol.

60. The method according to any one of claims 36 to 59, wherein the reaction is carried out in the presence of a co-solvent.

61. The method according to any one of claims 36 to 60, wherein the reaction is carried out in an aqueous co-solvent system.

62. The method according to claim 60 or 61, wherein the co-solvent is selected from dimethylsulfoxide (DMSO), and an alcohol, e.g., methanol, ethanol, n-propanol, isopropanol.

63. The method according to any one of claims 36 to 61, wherein the reaction is not carried out in the presence of dimethylsulfoxide (DMSO).

64. The method according to any one of claims 36 to 63, wherein the reaction is carried out at a temperature of 15 to 75 °C, e.g., 20 to 55 °C, e.g., 20 to 45 °C, e.g., 35 °C or 40 °C.

65. The method according to any one of claims 36 to 64, wherein the reaction is carried out at a pH of 5.0 to 10.0, e.g., 7.5-8.3.

66. The method according to any one of claims 36 to 65, wherein the concentration of the bicyclic ketone is up to about 150 g/L, e.g., at least about 5 g/L, at least about 10 g/L, at least about 20 g/L, at least about 50 g/L, at least about 100 g/L, or about 150 g/L.

67. The method according to any one of claims 36 to 66, wherein the concentration of the polypeptide is less than about 10 g/L, e.g., less than about 5 g/L, e.g., less than about 3 g/L, e.g., less than about 1 g/L.

68. The method according to any one of claims 36 to 67, wherein the solvent is present at a concentration of from about 20% to about 40% v/v.

69. The method according to any one of claims 36 to 68, wherein the method is carried out with whole cells that express the ketoreductase enzyme, or an extract or lysate of such cells.

70. The method according to any one of claims 36 to 69, wherein the method does not require glucose dehydrogenase (GDH)/glucose cofactor recycling.

71. The method according to any one of claims 36 to 70, wherein the ketoreductase is isolated and/or purified and the reduction reaction is carried out in the presence of a cofactor for the ketoreductase.

72. The method according to claim 71, wherein the cofactor comprises nicotinamide adenine dinucleotide (NADH) or nicotinamide adenine dinucleotide phosphate (NADPH).

73. The method according to claim 71 or 72, wherein the cofactor is present at a concentration of less than about 2 wt %, e.g., less than about 1 wt %, e.g., less than about 0.2 wt %, e.g., about 0.1 wt %, about 0.05 wt %, or about 0.03 wt %.

74. The method according to any one of claims 36 to 73, wherein method results in the product with a selectivity (% diastereomeric excess) greater than about 96%, 97%, 98%, 99%, 99.1%, 99.2%, 99.3%, 99.4%, 99.5%, 99.6%, 99.7%, 99.8% or 99.9% and/or a conversion % of at least about 85%, at least about 90%, at least about 95%, at least about 96%, at least about 97%, at least about 98%, at least about 99.0%, or 100%.

75. The method according to any one of claims 36 to 74, wherein at least about 85% of the substrate is reduced to product in less than about 20 hours and at least about 95% of the substrate is reduced to the product in less than about 30 hours.

76. A method for synthesizing a compound of formula (IC), in free form or in pharmaceutically acceptable salt form, 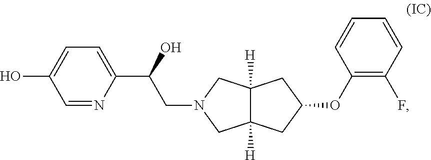 [SMILES: Oc1ccc([C@@H](O)CN2C[C@H]3C[C@H](Oc4ccccc4F)C[C@H]3C2)nc1]
the method comprising the step of contacting a compound of formula (IA)-I 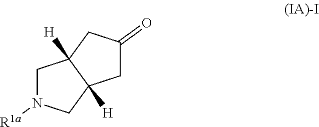
(Substrate) with a ketoreductase (KRED), under suitable reaction conditions, to produce a compound of formula (IB)-I 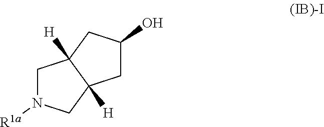
(product), wherein Ria is an amine protecting group, e.g., tert-butyloxycarbonyl (Boc) or N-carboxybenzyl (Cbz).

77. The method of claim 76, wherein the KRED is an engineered KRED.

78. The method of claim 76 or 77, wherein the KRED is defined according to any one of claims 1 to 35.

79. A method for reversing or increasing the diastereoselectivity of a ketoreductase (KRED) polypeptide towards the formation of (cis) alcohol product (e.g., (5s)-2) in a reduction reaction, the method comprising introducing one or more amino acid differences selected from Table 2, Table 4, Table 5, Table 6, or Table 7 relative to the amino acid sequence of the KRED polypeptide, wherein the one or more amino acid differences reverse or increase the diastereoselectivity of the KRED polypeptide towards the formation of (cis) alcohol product (e.g., (5s)-2) when used in a reduction reaction compared to the KRED polypeptide without the one or more amino acid differences.

80. The method according to claim 79, wherein the one or more amino acid differences are selected from the following positions: 17, 18, 40, 56, 94, 96, 106, 145, 173, 190, 194, 196, 198, 202, and/or 206, relative to the KRED polypeptide amino acid sequence.

81. The method according to any one of claims 79 or 80, wherein the one or more amino acid differences are selected from the following: X17S, X17T, X17G, X17A, X17R, X18G, X18S, X18R, X40H, X40R, X56V, X56S, X56T, X56D, X94A, X94V, X94L, X94I, X94W, X96Y, X106I, X106V, X106L, X106W, X106E, X145S, X145D, X145E, X173A, X173L, X173V, X173W, X173M, X173K, X173D, X190A, X194A, X194V, X194L, X194W, X194D, X194E, X194T, X194P, X196G, X198V, X198I, X198Y, X198D, X198P, X202A, X202G, X206Q, X206N and/or X206W, relative to the KRED polypeptide amino acid sequence.

82. The method according to any one of claims 79 to 81, wherein the one or more amino acid differences are selected from:
i) X17S, X17T, or X17G;
ii) X18S or X18R;
iii) X56S, X56T, or X56D;
iv) X94I;
iv) X106I, X106V, X106L, or X106W;
vi) X173A, X173W, X173M, or X173K;
vii) X194A, X194V, X194W, or X194T;
vii) X196G; and
viii) X198V, X198I, X198Y, or X198P;
relative to the KRED polypeptide amino acid sequence.

83. The method according to any one of claims 79 to 82, wherein the one or more amino acid differences are selected from: X94I, X96Y, X190A, X196G, X202A, X202G and/or X206W, relative to the KRED polypeptide amino acid sequence.

84. The method according to any one of claims 79 to 83, wherein the one or more amino acid differences are selected from: X18G, X40H, X56V, X94A, X106E, X145E, X145, X173D, X194P, X198D, X202A, and X206Q, relative to the KRED polypeptide amino acid sequence.

85. The method according to any one of claims 79 to 84, wherein the one or more amino acid differences are selected from: (i) a residue at position X190 that is not tyrosine; (ii) a residue at position X190 that is a non-aromatic residue; (iii) a residue at position X196 that is an aliphatic residue or a small amino acid residue; (iv) a residue at position X202 that is a small amino acid residue; and/or (v) a residue at position X206 that is not methionine, relative to the KRED polypeptide amino acid sequence.

86. The method according to any one of claims 79 to 85, wherein the KRED polypeptide is a wild-type Lactobacillus kefir, Lactobacillus brevis, or Lactobacillus minor ketoreductase polypeptide.

87. The method according to any one of claims 79 to 86, wherein the KRED polypeptide is a wild-type Lactobacillus kefir ketoreductase polypeptide.

88. The method according to any one of claims 79 to 87, wherein the KRED polypeptide is a wild-type Lactobacillus kefir ketoreductase polypeptide comprising an amino acid sequence of SEQ ID NO: 492.

89. The method according to any one of claims 79 to 85, wherein the KRED polypeptide is an engineered ketoreductase polypeptide.

90. The method according to claim 89, wherein the KRED polypeptide is an engineered ketoreductase polypeptide selected from Table 4, Table 5, or Table 6.

91. The method according to claim 89, wherein the KRED polypeptide is an engineered ketoreductase polypeptide selected from SEQ ID NO: 54, SEQ ID NO: 152, and SEQ ID NO: 256.

92. The method according to any one of claims 79 to 91, wherein the method results in a % conversion to (cis) alcohol product (e.g., (5s)-2) of at least about 85%, at least about 90%, at least about 95%, at least about 96%, at least about 97%, at least about 98%, at least about 99.0%, or 100%.

93. The method according to any one of claims 79 to 92, wherein the method results in a level of selectivity (% diastereomeric excess) of (cis) alcohol product (e.g., (5s)-2) of at least about 96.0%, at least about 96.5%, at least about 97.0%, at least about 97.5%, at least about 98.0%, at least about 98.5%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or 100%.

94. A composition comprising the engineered polypeptide of any one of claims 1 to 35.

95. The composition according to claim 94, further comprising the structural formula of Substrate and/or the compound of Product.

96. The engineered polypeptide according to any one of claims 1 to 35, wherein the polypeptide is immobilized on a solid support, via entrapment in a hydrogel (e.g., alginate, chitosan, carrageenan) or a matrix (e.g., polyacrylamide), or via carrier-free immobilization, e.g., cross-linking.

97. The engineered polypeptide of claim 96, wherein the polypeptide is immobilized to the solid support by a chemical bond (e.g., covalent or ionic bond), physical adsorption or affinity interactions.

98. The engineered polypeptide according to claim 96 or 97, wherein the solid support is organic or inorganic.

99. The engineered polypeptide according to any one of claims 96 to 98, wherein the solid support is selected from resin, silica, zeolite, charcoal, celite (e.g., diatomaceous earth), synthetic polymers (e.g., polymethacrylate or an anion exchange resin Amberlite), biopolymers (e.g., cellulose, chitosan, agarose, lignin or lignocellulose), controlled pore glass, magnetic nanoparticles, metal-organic frameworks, or DNA.

100. A polynucleotide encoding the engineered polypeptide according to any one of claims 1 to 35.

101. The polynucleotide according to claim 100, comprising a nucleic acid sequence listed in Table 4 (SEQ ID NOs: 3-51), Table 5 (SEQ ID NOs: 55-185), Table 6 (SEQ ID NOs: 187-389), or Table 7 (SEQ ID NOs: 391-489).

102. A polynucleotide encoding an engineered polypeptide, comprising a nucleic acid sequence that is at least about 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% identical to a nucleic acid sequence selected from the group consisting of: SEQ ID NO: 391, 393, 395, 405, 407, 419, 421, 423, 425, 427, 429, 435, 437, 461, 463, 467, 469, 473, 475, 479, 481, and 489.

103. An expression vector comprising the polynucleotide according to any one of claims 100 to 102 operably linked to control sequences suitable for directing expression of the encoded polypeptide in a host cell.

104. The expression vector according to claim 103, wherein the control sequence comprises a secretion signal.

105. A host cell comprising the polynucleotide according to any one of claims 100 to 102 or an expression vector according to claim 103 or 104.

106. A method of preparing an engineered ketoreductase polypeptide, the method comprising culturing the host cell of claim 105 under conditions suitable for gene expression and thereafter purifying and collecting the engineered polypeptide thereof from the cell culture.

107. A kit comprising the engineered polypeptide according to any one of claims 1 to 35, the composition according to any one of claims 94 or 95, the polynucleotide according to any one of claims 100 to 102, the expression vector according to claim 103 or 104, and/or the host cell according to claim 105.

108. A kit comprising the engineered polypeptide according to any one of claims 1 to 35, tert-butyl rel-(3aR,6aS)-5-oxohexahydrocyclopenta[c]pyrrole-2(1H)-carboxylate (Substrate), and a cofactor.

109. The kit of claim 108, wherein the cofactor is nicotinamide adenine dinucleotide (NADH) or nicotinamide adenine dinucleotide phosphate (NADPH).

110. The kit according to any one of claims 107 to 109, further comprising instructions for use of said engineered polypeptide, composition, polynucleotide, expression vector, and/or host cell.

111. A compound, which is 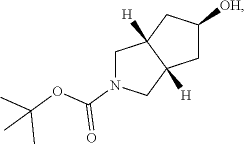 [SMILES: CC(C)(C)OC(=O)N1C[C@H]2C[C@H](O)C[C@H]2C1]
or a salt thereof.

112. The compound of claim 111, wherein the compound is present in a % diastereomeric excess (de) of at least about 85%, at least about 96%, or at least about 99%.

113. Use of the compound of claim 111 or 112, or a salt thereof, in the preparation of 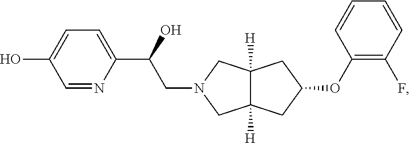 [SMILES: Oc1ccc([C@@H](O)CN2C[C@H]3C[C@H](Oc4ccccc4F)C[C@H]3C2)nc1]
in free form or in pharmaceutically acceptable salt form.


SEQUENCE LISTING (494 sequences)

SEQ ID NO: 1 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatgcaatcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 2 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGAIKTPLLDDLEGAEEMQSQRTKTPMGHIGEPNDIAWICVYLASDESKFATGAEFVVDGGYTAQ

SEQ ID NO: 3 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgcagctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatgcaatcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 4 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPQLGAYNASKGAVRIMSKSAALDCALKDYDVRVNTVHPGAIKTPLLDDLEGAEEMQSQRTKTPMGHIGEPNDIAWICVYLASDESKFATGAEFVVDGGYTAQ

SEQ ID NO: 5 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgcttctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatgcaatcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 6 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPLLGAYNASKGAVRIMSKSAALDCALKDYDVRVNTVHPGAIKTPLLDDLEGAEEMQSQRTKTPMGHIGEPNDIAWICVYLASDESKFATGAEFVVDGGYTAQ

SEQ ID NO: 7 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattttggtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatgcaatcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 8 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGILVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGAIKTPLLDDLEGAEEMQSQRTKTPMGHIGEPNDIAWICVYLASDESKFATGAEFVVDGGYTAQ

SEQ ID NO: 9 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgaggctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatgcaatcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 10 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPRLGAYNASKGAVRIMSKSAALDCALKDYDVRVNTVHPGAIKTPLLDDLEGAEEMQSQRTKTPMGHIGEPNDIAWICVYLASDESKFATGAEFVVDGGYTAQ

SEQ ID NO: 11 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatggcgtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 12 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGAIKTPLLDDLEGAEEMASQRTKTPMGHIGEPNDIAWICVYLASDESKFATGAEFVVDGGYTAQ

SEQ ID NO: 13 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgagggggttgtaggcgatccgacgctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatgcaatcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 14 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGVVGDPTLGAYNASKGAVRIMSKSAALDCALKDYDVRVNTVHPGAIKTPLLDDLEGAEEMQSQRTKTPMGHIGEPNDIAWICVYLASDESKFATGAEFVVDGGYTAQ

SEQ ID NO: 15 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggcgccatcaagacctgtctgctcgatgatctggaaggtgctgaggaaatgcaatcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 16 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGAIKTCLLDDLEGAEEMQSQRTKTPMGHIGEPNDIAWICVYLASDESKFATGAEFVVDGGYTAQ

SEQ ID NO: 17 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatgttgtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 18 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGAIKTPLLDDLEGAEEMLSQRTKTPMGHIGEPNDIAWICVYLASDESKFATGAEFVVDGGYTAQ

SEQ ID NO: 19 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagttgctaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatgcaatcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 20 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVAKSVEDTTTEEWRKLLSVNLDGVFFGTRLGIQRMKNKGLGASIINMSSIEGFVGDPTLGAYNASKGAVRIMSKSAALDCALKDYDVRVNTVHPGAIKTPLLDDLEGAEEMQSQRTKTPMGHIGEPNDIAWICVYLASDESKFATGAEFVVDGGYTAQ

SEQ ID NO: 21 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatcagctggaaggtgctgaggaaatgcaatcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 22 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGAIKTPLLDQLEGAEEMQSQRTKTPMGHIGEPNDIAWICVYLASDESKFATGAEFVVDGGYTAQ

SEQ ID NO: 23 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcggtactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagttgggaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatgcaatcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 24 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVGKSVEDTTTEEWRKLLSVNLDGVFFGTRLGIQRMKNKGLGASIINMSSIEGFVGDPTLGAYNASKGAVRIMSKSAALDCALKDYDVRVNTVHPGAIKTPLLDDLEGAEEMQSQRTKTPMGHIGEPNDIAWICVYLASDESKFATGAEFVVDGGYTAQ

SEQ ID NO: 25 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatgcaatcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 26 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGAIKTPLLDDLEGAEEMQSQRTKTPMGHIGEPNDIAWICVYLASDESKFATGAEFVVDGGYTAQ

SEQ ID NO: 27 - DNA, 759 bp, synthetic construct
  misc_feature 1..759: note = Engineered variants of ketoreductase from Lactobacillus kefir
  source 1..759: mol_type = other DNA
atgaccgatcgtctgaagagcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaatgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatgcaatcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 28 - protein, 252 aa, synthetic construct
  REGION 1..252: note = Engineered variants of ketoreductase from Lactobacillus kefir
MTDRLKSKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGAIKTPLLDDLEGAEEMQSQRTKTPMGHIGEPNDIAWICVYLASDESKFATGAEFVVDGGYTAQ

SEQ ID NO: 29 - DNA, 759 bp, synthetic construct
  misc_feature 1..759: note = Engineered variants of ketoreductase from Lactobacillus kefir
  source 1..759: mol_type = other DNA
atgaccgatcgtctgaagagcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagtgggctggtaggcgatccgacgctgggggcatacaacgcttccaagggggcggtacgtatcatgtcgaaaagcgcagcgctggattgcgcactgaaggactacgatgtgcgtgtcaacacagtacatccgggccctatcaagaccccgctgctcgatgatctggaaggtgctgaggaaatgcaatcacagcgtacgctgacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 30 - protein, 252 aa, synthetic construct
  REGION 1..252: note = Engineered variants of ketoreductase from Lactobacillus kefir
MTDRLKSKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGPIKTPLLDDLEGAEEMQSQRTLTPMGHIGEPNDIAWICVYLASDESKFATGAEFVVDGGYTAQ

SEQ ID NO: 31 - DNA, 759 bp, synthetic construct
  misc_feature 1..759: note = Engineered variants of ketoreductase from Lactobacillus kefir
  source 1..759: mol_type = other DNA
atgaccgatcgtctgaagagcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcacaaactgctgtccgttaatctggatggcgtttttttcggcacccgtctgggcattcagcgcatgaaaaataaaggcttgggcgctagcatcatcaatatgagcagtattagtgggctggtaggcgatccgacgctgggggcatacaatgcttccaagggggcggtacgtatcatgtcgaaaagcgcagcgctggattgcgcactgaaggactacgatgtgcgtgtcaacacagtacatccgggccctatcaagaccccgctgctcgatgatctggaaggtgctgaggaaatgcaatcacagcgtacgctgacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 32 - protein, 252 aa, synthetic construct
  REGION 1..252: note = Engineered variants of ketoreductase from Lactobacillus kefir
MTDRLKSKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWHKLLSVNLDGVFFGTRLGIQRMKNKGLGASIINMSSISGLVGDPTLGAYNASKGAVRIMSKSAALDCALKDYDVRVNTVHPGPIKTPLLDDLEGAEEMQSQRTLTPMGHIGEPNDIAWICVYLASDESKFATGAEFVVDGGYTAQ

SEQ ID NO: 33 - DNA, 759 bp, synthetic construct
  misc_feature 1..759: note = Engineered variants of ketoreductase from Lactobacillus kefir
  source 1..759: mol_type = other DNA
atgaccgatcgtctgaagagcaaagtagccatcgtaaccggcgggactctgggtatcggtttggcaatcgccgataaatttgtagaggagggtgcgaaagtagttattactggtcgtaaggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagttagcaaaagcgttgaagacactaccacggaggaatggcgcaaactgctgtccgttaatctggatagcgtttttttcggcacccgtctgggcattcagcgcatgaaaaataaaggcttgggcgctagcatcatcaatatgagcagtattagtgggctggtaggcgatccgacgctgggggcatacaatgcttccaagggggcggtacgtatcatgtcgaaaagcgcagcgctggattgcgcactgaaggactacgatgtgcgtgtcaacacagtacatccgggccctatcaagaccccgctgctcgatgatctggaaggtgctgaggaaatgcaatcacagcgtacgctgacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 34 - protein, 252 aa, synthetic construct
  REGION 1..252: note = Engineered variants of ketoreductase from Lactobacillus kefir
MTDRLKSKVAIVTGGTLGIGLAIADKFVEEGAKVVITGRKADVGEKAAKSIGGTDVIRFVQHDASDEAGWTKLFDTTEEAFGPVTTVVNNAGIAVSKSVEDTTTEEWRKLLSVNLDSVFFGTRLGIQRMKNKGLGASIINMSSISGLVGDPTLGAYNASKGAVRIMSKSAALDCALKDYDVRVNTVHPGPIKTPLLDDLEGAEEMQSQRTLTPMGHIGEPNDIAWICVYLASDESKFATGAEFVVDGGYTAQ

SEQ ID NO: 35 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggccctatcaagaccccgctgctcgatgatctggaaggtgctgaggaaatgaattcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 36 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGPIKTPLLDDLEGAEEMNSQRTKTPMGHIGEPNDIAWICVYLASDESKFATGAEFVVDGGYTAQ

SEQ ID NO: 37 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtatcgaggggctggtaggcgatccgacgctgggggcatacaacgcttccaagggggcggtacgtatcatgtcgaaaagcgcagcgctggattgcgcactgaaggactacgatgtgcgtgtcaacacagtacatccgggcgccatcaagaccccgctgctggatgatctggaaggtgctgaggaaatgcagtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 38 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLLDDLEGAEEMQSQRTKTPMGHIGEPNDIAWICVYLASDESKFATGAEFVVDGGYTAQ

SEQ ID NO: 39 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtatcgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggcgtcatcaagaccccgctgattgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 40 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGVIKTPLIDDLEGAEEMWSQRTKTPMGHIGEPNDIAWICVYLASDESKFATGAEFVVDGGYTAQ

SEQ ID NO: 41 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtatcgaggggctggtaggcgatccgacgctgggggcatacaacgcttccaagggggcggtacgtatcatgtcgaaaagcgcagcgctggattgcgcactgaaggactacgatgtgcgtgtcaacacagtacatccgggctgtatcaagaccccgctgattgatgatctggaaggtgctgaggaaatgcagtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 42 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGLVGDPTLGAYNASKGAVRIMSKSAALDCALKDYDVRVNTVHPGCIKTPLIDDLEGAEEMQSQRTKTPMGHIGEPNDIAWICVYLASDESKFATGAEFVVDGGYTAQ

SEQ ID NO: 43 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtaaagcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggctgtatcaagaccccgctgattgatgatctggaaggtgctgaggaaatgaattcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 44 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRKADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGCIKTPLIDDLEGAEEMNSQRTKTPMGHIGEPNDIAWICVYLASDESKFATGAEFVVDGGYTAQ

SEQ ID NO: 45 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggcgccatcaagaccccgctgctcgatgatctggaaggtgctgaggaaatgaattcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 46 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGAIKTPLLDDLEGAEEMNSQRTKTPMGHIGEPNDIAWICVYLASDESKFATGAEFVVDGGYTAQ

SEQ ID NO: 47 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtatcgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggctgtatcaagaccccgctgattgatgatctggaaggtgctgaggaaatgaattcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 48 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGCIKTPLIDDLEGAEEMNSQRTKTPMGHIGEPNDIAWICVYLASDESKFATGAEFVVDGGYTAQ

SEQ ID NO: 49 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtaaagcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtatcgaggggttcgtaggcgatccgacgctgggggcatacaacgcttccaagggggcggtacgtatcatgtcgaaaagcgcagcgctggattgcgcactgaaggactacgatgtgcgtgtcaacacagtacatccgggccctatcaagaccccgctgctcgatgatctggaaggtgctgaggaaatgaattcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 50 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRKADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGPIKTPLLDDLEGAEEMNSQRTKTPMGHIGEPNDIAWICVYLASDESKFATGAEFVVDGGYTAQ

SEQ ID NO: 51 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttccaaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtatcgaggggctggtaggcgatccgacgctgggggcatacaacgcttccaagggggcggtacgtatcatgtcgaaaagcgcagcgctggattgcgcactgaaggactacgatgtgcgtgtcaacacagtacatccgggccctatcaagaccccgctgattgatgatgtagaaggtgctgaggaaatgaattcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 52 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGLVGDPTLGAYNASKGAVRIMSKSAALDCALKDYDVRVNTVHPGPIKTPLIDDVEGAEEMNSQRTKTPMGHIGEPNDIAWICVYLASDESKFATGAEFVVDGGYTAQ

SEQ ID NO: 53 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 54 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 55 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggatttgggtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtatcagtgggctcgtaggcgacccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgccgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 56 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIWVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 57 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggatttgggtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtggcgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 58 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIWVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGGEEMWSQRTKTPMGHIGEPNDIAWICVYLASDESKFATGAEFVVDGGYTAQ

SEQ ID NO: 59 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgatgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 60 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIDGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 61 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcaatttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 62 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAIYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 63 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactacgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 64 - protein, 252 aa, synthetic construct
  REGION 1..252: note = Engineered variants of ketoreductase from Lactobacillus kefir
MTDRLKGKVAIVTGGTTGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 65 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatccataccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 66 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIHTPLVDDLEGAEEMWSQRTKTPMGHIGEPNDIAWICVYLASDESKFATGAEFVVDGGYTAQ

SEQ ID NO: 67 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaacgaatgacatcgcatggatctgtgtgtacctggcatctgacgaatcgaaatttgcgacgggtgcagaatttgtggtcgacggcgggtataccgcacagtga

SEQ ID NO: 68 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGETNDIAWICVYLASDESKFATGAEFVVDGGYTAQ

SEQ ID NO: 69 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaactgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 70 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEELWSQRTKTPMGHIGEPNDIAWICVYLASDESKFATGAEFVVDGGYTAQ

SEQ ID NO: 71 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatccggaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 72 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIRTPLVDDLEGAEEMWSQRTKTPMGHIGEPNDIAWICVYLASDESKFATGAEFVVDGGYTAQ

SEQ ID NO: 73 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatccgctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 74 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDPLEGAEEMWSQRTKTPMGHIGEPNDIAWICVYLASDESKFATGAEFVVDGGYTAQ

SEQ ID NO: 75 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagacccttctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 76 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTLLVDDLEGAEEMWSQRTKTPMGHIGEPNDIAWICVYLASDESKFATGAEFVVDGGYTAQ

SEQ ID NO: 77 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccgatctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 78 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTDLVDDLEGAEEMWSQRTKTPMGHIGEPNDIAWICVYLASDESKFATGAEFVVDGGYTAQ

SEQ ID NO: 79 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccactctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 80 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTTLVDDLEGAEEMWSQRTKTPMGHIGEPNDIAWICVYLASDESKFATGAEFVVDGGYTAQ

SEQ ID NO: 81 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccgagctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 82 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTELVDDLEGAEEMWSQRTKTPMGHIGEPNDIAWICVYLASDESKFATGAEFVVDGGYTAQ

SEQ ID NO: 83 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgttctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 84 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDVLEGAEEMWSQRTKTPMGHIGEPNDIAWICVYLASDESKFATGAEFVVDGGYTAQ

SEQ ID NO: 85 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagacctggctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 86 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTWLVDDLEGAEEMWSQRTKTPMGHIGEPNDIAWICVYLASDESKFATGAEFVVDGGYTAQ

SEQ ID NO: 87 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttatattagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 88 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYISVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 89 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagcggccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 90 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKRPLVDDLEGAEEMWSQRTKTPMGHIGEPNDIAWICVYLASDESKFATGAEFVVDGGYTAQ

SEQ ID NO: 91 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgagtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 92 - protein, 252 aa, synthetic construct
  REGION 1..252: note = Engineered variants of ketoreductase from Lactobacillus kefir
MTDRLKGKVAIVTGGTLSIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 93 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaggggttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 94 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKGVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 95 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttatatgagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 96 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYMSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 97 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtctggaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 98 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGLEEMWSQRTKTPMGHIGEPNDIAWICVYLASDESKFATGAEFVVDGGYTAQ

SEQ ID NO: 99 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcggggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 100 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 101 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcaatgtataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 102 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAMYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 103 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcaccttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 104 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAPYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 105 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaatattggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 106 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEYWSQRTKTPMGHIGEPNDIAWICVYLASDESKFATGAEFVVDGGYTAQ

SEQ ID NO: 107 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcagcgcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 108 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSARTKTPMGHIGEPNDIAWICVYLASDESKFATGAEFVVDGGYTAQ

SEQ ID NO: 109 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactgggggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 110 - protein, 252 aa, synthetic construct
  REGION 1..252: note = Engineered variants of ketoreductase from Lactobacillus kefir
MTDRLKGKVAIVTGGTGGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 111 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatagtacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 112 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDSTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 113 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaagttaatgacatcgcatggatctgtgtgtacctggcatctgacgaatcgaaatttgcgacgggtgcagaatttgtggtcgacggcgggtataccgcacagtga

SEQ ID NO: 114 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEVNDIAWICVYLASDESKFATGAEFVVDGGYTAQ

SEQ ID NO: 115 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatattctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 116 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDILEGAEEMWSQRTKTPMGHIGEPNDIAWICVYLASDESKFATGAEFVVDGGYTAQ

SEQ ID NO: 117 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaaggggccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 118 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKGPLVDDLEGAEEMWSQRTKTPMGHIGEPNDIAWICVYLASDESKFATGAEFVVDGGYTAQ

SEQ ID NO: 119 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagcagccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 120 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKQPLVDDLEGAEEMWSQRTKTPMGHIGEPNDIAWICVYLASDESKFATGAEFVVDGGYTAQ

SEQ ID NO: 121 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggagtggtggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 122 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEWWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 123 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctgggtggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 124 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLGGAEEMWSQRTKTPMGHIGEPNDIAWICVYLASDESKFATGAEFVVDGGYTAQ

SEQ ID NO: 125 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcgataccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 126 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIDTPLVDDLEGAEEMWSQRTKTPMGHIGEPNDIAWICVYLASDESKFATGAEFVVDGGYTAQ

SEQ ID NO: 127 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgcgtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 128 - protein, 252 aa, synthetic construct
  REGION 1..252: note = Engineered variants of ketoreductase from Lactobacillus kefir
MTDRLKGKVAIVTGGTLRIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 129 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcatgaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 130 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIMTPLVDDLEGAEEMWSQRTKTPMGHIGEPNDIAWICVYLASDESKFATGAEFVVDGGYTAQ

SEQ ID NO: 131 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacgttgcatggatctgtgtgtacctggcatctgacgaatcgaaatttgcgacgggtgcagaatttgtggtcgacggcgggtataccgcacagtga

SEQ ID NO: 132 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDVAWICVYLASDESKFATGAEFVVDGGYTAQ

SEQ ID NO: 133 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggagttgtggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 134 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTELWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 135 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacggtgacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 136 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTVTPMGHIGEPNDIAWICVYLASDESKFATGAEFVVDGGYTAQ

SEQ ID NO: 137 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgattctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 138 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPILGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 139 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccgttctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 140 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTVLVDDLEGAEEMWSQRTKTPMGHIGEPNDIAWICVYLASDESKFATGAEFVVDGGYTAQ

SEQ ID NO: 141 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggtgtggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 142 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEVWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 143 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggggtacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 144 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGGYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 145 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttatcggagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 146 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYRSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 147 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgcctgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 148 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLPDDLEGAEEMWSQRTKTPMGHIGEPNDIAWICVYLASDESKFATGAEFVVDGGYTAQ

SEQ ID NO: 149 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcaacgcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 150 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSTRTKTPMGHIGEPNDIAWICVYLASDESKFATGAEFVVDGGYTAQ

SEQ ID NO: 151 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 152 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 153 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcctgccgaatgacatcgcatggatctgtgtgtacctggcatctgacgaatcgaaatttgcgacgggtgcagaatttgtggtcgacggcgggtataccgcacagtga

SEQ ID NO: 154 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGLPNDIAWICVYLASDESKFATGAEFVVDGGYTAQ

SEQ ID NO: 155 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggaaggcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 156 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGKAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 157 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgttgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 158 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIVVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 159 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgagtgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 160 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLSDDLEGAEEMWSQRTKTPMGHIGEPNDIAWICVYLASDESKFATGAEFVVDGGYTAQ

SEQ ID NO: 161 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaatgggctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 162 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEWAEEMWSQRTKTPMGHIGEPNDIAWICVYLASDESKFATGAEFVVDGGYTAQ

SEQ ID NO: 163 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcagtgcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 164 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSVRTKTPMGHIGEPNDIAWICVYLASDESKFATGAEFVVDGGYTAQ

SEQ ID NO: 165 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactgcgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 166 - protein, 252 aa, synthetic construct
  REGION 1..252: note = Engineered variants of ketoreductase from Lactobacillus kefir
MTDRLKGKVAIVTGGTAGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 167 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatctggaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 168 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIWTPLVDDLEGAEEMWSQRTKTPMGHIGEPNDIAWICVYLASDESKFATGAEFVVDGGYTAQ

SEQ ID NO: 169 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggagatttggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 170 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEIWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDIAWICVYLASDESKFATGAEFVVDGGYTAQ

SEQ ID NO: 171 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccattaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctgtggggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 172 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSINLDGVFFGTRLGIQRMKNKGLGASIINMSSISGLVGDPTLGAYNASKGAVRIMSKSAALDCALKDYDVRVNTVHPGAIKTPLVDDLWGAEEMWSQRTKTPMGHIGEPNDIAWICVYLASDESKFATGAEFVVDGGYTAQ

SEQ ID NO: 173 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaattttggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 174 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEFWSQRTKTPMGHIGEPNDIAWICVYLASDESKFATGAEFVVDGGYTAQ

SEQ ID NO: 175 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccgctctggtcgatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 176 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTALVDDLEGAEEMWSQRTKTPMGHIGEPNDIAWICVYLASDESKFATGAEFVVDGGYTAQ

SEQ ID NO: 177 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaaatgtggtcacagcgtacgaaaacccctatgggccacattggcgaaccgaatgactgtgcatggatctgtgtgtacctggcatctgacgaatcgaaatttgcgacgggtgcagaatttgtggtcgacggcgggtataccgcacagtga

SEQ ID NO: 178 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEMWSQRTKTPMGHIGEPNDCAWICVYLASDESKFATGAEFVVDGGYTAQ

SEQ ID NO: 179 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgattatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 180 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDYLEGAEEMWSQRTKTPMGHIGEPNDIAWICVYLASDESKFATGAEFVVDGGYTAQ

SEQ ID NO: 181 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgcagctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaatggtggtcacagcgtacgttgacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 182 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPQLGAYNASKGAVRIMSKSAALDCALKDYDVRVNTVHPGAIKTPLVDDLEGAEEWWSQRTLTPMGHIGEPNDIAWICVYLASDESKFATGAEFVVDGGYTAQ

SEQ ID NO: 183 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaatggtggtcacagcgtacgttgacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 184 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEEWWSQRTLTPMGHIGEPNDIAWICVYLASDESKFATGAEFVVDGGYTAQ

SEQ ID NO: 185 - DNA, 759 bp, synthetic construct
  misc_feature 1..759: note = Engineered variants of ketoreductase from Lactobacillus kefir
  source 1..759: mol_type = other DNA
atgaccgatcgtctgaagagcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggtcgatgatctggaaggtgctgaggaactgtggtcacagcgtacgaagacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 186 - protein, 252 aa, synthetic construct
  REGION 1..252: note = Engineered variants of ketoreductase from Lactobacillus kefir
MTDRLKSKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLVDDLEGAEELWSQRTKTPMGHIGEPNDIAWICVYLASDESKFATGAEFVVDGGYTAQ

SEQ ID NO: 187 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaatactggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 188 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEYWSQRTKTPMGHIGEPNDIAWICVYLASDESKFATGAEFVVDGGYTAQ

SEQ ID NO: 189 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcggtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 190 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 191 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcggtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaactgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 192 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEELWSQRTKTPMGHIGEPNDIAWICVYLASDESKFATGAEFVVDGGYTAQ

SEQ ID NO: 193 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattatcgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaatggtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 194 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEWWSQRTKTPMGHIGEPNDIAWICVYLASDESKFATGAEFVVDGGYTAQ

SEQ ID NO: 195 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtaaggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagacgtggctggcggatgatctggaaggtgctgaggaatactggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 196 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRKADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTWLADDLEGAEEYWSQRTKTPMGHIGEPNDIAWICVYLASDESKFATGAEFVVDGGYTAQ

SEQ ID NO: 197 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattatcgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaactgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 198 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEELWSQRTKTPMGHIGEPNDIAWICVYLASDESKFATGAEFVVDGGYTAQ

SEQ ID NO: 199 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcggtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaatactggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 200 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEEYWSQRTKTPMGHIGEPNDIAWICVYLASDESKFATGAEFVVDGGYTAQ

SEQ ID NO: 201 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaactgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 202 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEELWSQRTKTPMGHIGEPNDIAWICVYLASDESKFATGAEFVVDGGYTAQ

SEQ ID NO: 203 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcggtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaactgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 204 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEELWSQRTKTPMGHIGEPNDIAWICVYLASDESKFATGAEFVVDGGYTAQ

SEQ ID NO: 205 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagacgtggctggcggatcctctggaaggtgctgaggaatggtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 206 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTWLADPLEGAEEWWSQRTKTPMGHIGEPNDIAWICVYLASDESKFATGAEFVVDGGYTAQ

SEQ ID NO: 207 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 208 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 209 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgacgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 210 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIDGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 211 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaatggtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 212 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEWWSQRTKTPMGHIGEPNDIAWICVYLASDESKFATGAEFVVDGGYTAQ

SEQ ID NO: 213 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtatttttgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagacgctgctggcggatgatctggaaggtgctgaggaatactggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 214 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIFGLVGDPTLGAYNASKGAVRIMSKSAALDCALKDYDVRVNTVHPGAIKTLLADDLEGAEEYWSQRTKTPMGHIGEPNDIAWICVYLASDESKFATGAEFVVDGGYTAQ

SEQ ID NO: 215 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgcgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgacgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagacgtggctggcggatgatctggaaggtgctgaggaatactggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 216 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIDGLVGDPTLGAYNASKGAVRIMSKSAALDCALKDYDVRVNTVHPGAIKTWLADDLEGAEEYWSQRTKTPMGHIGEPNDIAWICVYLASDESKFATGAEFVVDGGYTAQ

SEQ ID NO: 217 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaatggtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 218 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEEWWSQRTKTPMGHIGEPNDIAWICVYLASDESKFATGAEFVVDGGYTAQ

SEQ ID NO: 219 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattatcgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgacgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 220 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSIDGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 221 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtaaggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagacctcgctgggggatcctctggaaggtgctgaggaatactggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 222 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRKADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTSLGDPLEGAEEYWSQRTKTPMGHIGEPNDIAWICVYLASDESKFATGAEFVVDGGYTAQ

SEQ ID NO: 223 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtaaggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcggtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaggggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaatactggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 224 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRKADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEEYWSQRTKTPMGHIGEPNDIAWICVYLASDESKFATGAEFVVDGGYTAQ

SEQ ID NO: 225 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgattcgattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 226 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDSIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 227 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggttttgcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 228 - protein, 252 aa, synthetic construct
  REGION 1..252: note = Engineered variants of ketoreductase from Lactobacillus kefir
MTDRLKGKVAIVTGGTLGIGFAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 229 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaatgtactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 230 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVECTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 231 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttaaggacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 232 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVKDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 233 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctgatgtgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 234 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALMCALKDYDVRVNTVHPGAIKTPLGDDLEGAEEMWSQRTKTPMGHIGEPNDIAWICVYLASDESKFATGAEFVVDGGYTAQ

SEQ ID NO: 235 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaatttactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 236 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEFTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 237 - DNA, 759 bp, synthetic construct
  misc_feature 1..759: note = Engineered variants of ketoreductase from Lactobacillus kefir
  source 1..759: mol_type = other DNA
atgaccgatcgtctgaagcgtaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 238 - protein, 252 aa, synthetic construct
  REGION 1..252: note = Engineered variants of ketoreductase from Lactobacillus kefir
MTDRLKRKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 239 - DNA, 759 bp, synthetic construct
  misc_feature 1..759: note = Engineered variants of ketoreductase from Lactobacillus kefir
  source 1..759: mol_type = other DNA
atgaccgatcgtctgaagaagaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 240 - protein, 252 aa, synthetic construct
  REGION 1..252: note = Engineered variants of ketoreductase from Lactobacillus kefir
MTDRLKKKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 241 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaacgggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 242 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGERAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 243 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggggatggatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 244 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGMDLEGAEEMWSQRTKTPMGHIGEPNDIAWICVYLASDESKFATGAEFVVDGGYTAQ

SEQ ID NO: 245 - DNA, 759 bp, synthetic construct
  misc_feature 1..759: note = Engineered variants of ketoreductase from Lactobacillus kefir
  source 1..759: mol_type = other DNA
atgtttgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 246 - protein, 252 aa, synthetic construct
  REGION 1..252: note = Engineered variants of ketoreductase from Lactobacillus kefir
MFDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 247 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtcagaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 248 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGQKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 249 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctgtggtgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 250 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALWCALKDYDVRVNTVHPGAIKTPLGDDLEGAEEMWSQRTKTPMGHIGEPNDIAWICVYLASDESKFATGAEFVVDGGYTAQ

SEQ ID NO: 251 - DNA, 759 bp, synthetic construct
  misc_feature 1..759: note = Engineered variants of ketoreductase from Lactobacillus kefir
  source 1..759: mol_type = other DNA
atgaccgatcgtctgaagtgtaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 252 - protein, 252 aa, synthetic construct
  REGION 1..252: note = Engineered variants of ketoreductase from Lactobacillus kefir
MTDRLKCKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 253 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaacagactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 254 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEQTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 255 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 256 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 257 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatcgtggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 258 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADRGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 259 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgatattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 260 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDDIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 261 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactatgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 262 - protein, 252 aa, synthetic construct
  REGION 1..252: note = Engineered variants of ketoreductase from Lactobacillus kefir
MTDRLKGKVAIVTGGTMGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 263 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatactattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 264 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDTIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 265 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggccgtgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 266 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGRDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 267 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtttgaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 268 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGLKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 269 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaacggactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 270 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVERTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 271 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggttctgcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 272 - protein, 252 aa, synthetic construct
  REGION 1..252: note = Engineered variants of ketoreductase from Lactobacillus kefir
MTDRLKGKVAIVTGGTLGIGSAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 273 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgctgacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 274 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVADTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 275 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaaaatactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 276 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVENTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 277 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggtttgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 278 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALVCALKDYDVRVNTVHPGAIKTPLGDDLEGAEEMWSQRTKTPMGHIGEPNDIAWICVYLASDESKFATGAEFVVDGGYTAQ

SEQ ID NO: 279 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccacgaaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 280 - protein, 252 aa, synthetic construct
  REGION 1..252: note = Engineered variants of ketoreductase from Lactobacillus kefir
MTDRLKGKVAIVTGGTLGIGLAIATKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 281 - DNA, 759 bp, synthetic construct
  misc_feature 1..759: note = Engineered variants of ketoreductase from Lactobacillus kefir
  source 1..759: mol_type = other DNA
atgaccgatcgtctgaagactaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 282 - protein, 252 aa, synthetic construct
  REGION 1..252: note = Engineered variants of ketoreductase from Lactobacillus kefir
MTDRLKTKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 283 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtcggaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 284 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGRKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 285 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacggggctgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 286 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTGLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 287 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactgagacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 288 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTETEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 289 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaaagtactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 290 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVESTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 291 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacacttggacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 292 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTWTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 293 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaaactactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 294 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVETTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 295 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgactctgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 296 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTTLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 297 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaaggtactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 298 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEGTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 299 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgccacgcggatgtaggtgaaaaggccgccaaatcaatcggcggcactgatgttattcgctttgtccagcacgatgcatccgatgaagcaggctggacgaaactgttcgacaagaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 300 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDKTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 301 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagtgactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 302 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEVTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 303 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggtttaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 304 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWFKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 305 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaacttactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 306 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVELTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 307 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagctactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 308 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEATTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 309 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggggagggatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 310 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGRDLEGAEEMWSQRTKTPMGHIGEPNDIAWICVYLASDESKFATGAEFVVDGGYTAQ

SEQ ID NO: 311 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaagtcgtacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 312 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKSYDVRVNTVHPGAIKTPLGDDLEGAEEMWSQRTKTPMGHIGEPNDIAWICVYLASDESKFATGAEFVVDGGYTAQ

SEQ ID NO: 313 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttggcggctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 314 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLAASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 315 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgcgggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 316 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALRDYDVRVNTVHPGAIKTPLGDDLEGAEEMWSQRTKTPMGHIGEPNDIAWICVYLASDESKFATGAEFVVDGGYTAQ

SEQ ID NO: 317 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcaaagaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 318 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCAKKDYDVRVNTVHPGAIKTPLGDDLEGAEEMWSQRTKTPMGHIGEPNDIAWICVYLASDESKFATGAEFVVDGGYTAQ

SEQ ID NO: 319 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggcttgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 320 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALACALKDYDVRVNTVHPGAIKTPLGDDLEGAEEMWSQRTKTPMGHIGEPNDIAWICVYLASDESKFATGAEFVVDGGYTAQ

SEQ ID NO: 321 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcagggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 322 - protein, 252 aa, synthetic construct
  REGION 1..252: note = Engineered variants of ketoreductase from Lactobacillus kefir
MTDRLKGKVAIVTGGTQGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 323 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttagggacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 324 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVRDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 325 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctgctgtgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 326 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALLCALKDYDVRVNTVHPGAIKTPLGDDLEGAEEMWSQRTKTPMGHIGEPNDIAWICVYLASDESKFATGAEFVVDGGYTAQ

SEQ ID NO: 327 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgtgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 328 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 329 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactatgggtatcggtttggcaatcgccgataaatttgtagaggagggtgcgaaagtagttattactggtcgtaaggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 330 - protein, 252 aa, synthetic construct
  REGION 1..252: note = Engineered variants of ketoreductase from Lactobacillus kefir
MTDRLKGKVAIVTGGTMGIGLAIADKFVEEGAKVVITGRKADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 331 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgtgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 332 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 333 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccactctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 334 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTTLGDDLEGAEEMWSQRTKTPMGHIGEPNDIAWICVYLASDESKFATGAEFVVDGGYTAQ

SEQ ID NO: 335 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtaaggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatctggaccactctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 336 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRKADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIWTTLGDDLEGAEEMWSQRTKTPMGHIGEPNDIAWICVYLASDESKFATGAEFVVDGGYTAQ

SEQ ID NO: 337 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccgagctgggggatgacctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 338 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTELGDDLEGAEEMWSQRTKTPMGHIGEPNDIAWICVYLASDESKFATGAEFVVDGGYTAQ

SEQ ID NO: 339 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtaaggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagacccctctgggggatgatctggaaggtgcggaggaattctggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 340 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRKADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEFWSQRTKTPMGHIGEPNDIAWICVYLASDESKFATGAEFVVDGGYTAQ

SEQ ID NO: 341 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 342 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 343 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtaaggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccgagctgggggatgatctggaaggtgcggaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 344 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRKADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTELGDDLEGAEEMWSQRTKTPMGHIGEPNDIAWICVYLASDESKFATGAEFVVDGGYTAQ

SEQ ID NO: 345 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 346 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 347 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtcgtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgacctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 348 - protein, 252 aa, synthetic construct
  REGION 1..252: note = Engineered variants of ketoreductase from Lactobacillus kefir
MTDRLKGKVAIVTGGTRRIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 349 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcgtgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccgagctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 350 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTELGDDLEGAEEMWSQRTKTPMGHIGEPNDIAWICVYLASDESKFATGAEFVVDGGYTAQ

SEQ ID NO: 351 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcgtgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccgagctgggggatgatctggaaggtgcggaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 352 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTELGDDLEGAEEMWSQRTKTPMGHIGEPNDIAWICVYLASDESKFATGAEFVVDGGYTAQ

SEQ ID NO: 353 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactatgggtatcggtttggcaatcgccgataaatttgtagaggagggtgcgaaagtagttattactggtcgtcgtgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 354 - protein, 252 aa, synthetic construct
  REGION 1..252: note = Engineered variants of ketoreductase from Lactobacillus kefir
MTDRLKGKVAIVTGGTMGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 355 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccgagctgggggatgatctggaaggtgcggaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 356 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTELGDDLEGAEEMWSQRTKTPMGHIGEPNDIAWICVYLASDESKFATGAEFVVDGGYTAQ

SEQ ID NO: 357 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtaaggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgaagctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 358 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRKADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPKLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 359 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccactctgggggatgatctggaaggtgcggaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 360 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTTLGDDLEGAEEMWSQRTKTPMGHIGEPNDIAWICVYLASDESKFATGAEFVVDGGYTAQ

SEQ ID NO: 361 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactatgggtatcggtttggcaatcgccgataaatttgtagaggagggtgcgaaagtagttattactggtcgtcatgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 362 - protein, 252 aa, synthetic construct
  REGION 1..252: note = Engineered variants of ketoreductase from Lactobacillus kefir
MTDRLKGKVAIVTGGTMGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 363 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcgtgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 364 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 365 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactatgggtatcggtttggcaatcgccgataaatttgtagaggagggtgcgaaagtagttattactggtcgtaaggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 366 - protein, 252 aa, synthetic construct
  REGION 1..252: note = Engineered variants of ketoreductase from Lactobacillus kefir
MTDRLKGKVAIVTGGTMGIGLAIADKFVEEGAKVVITGRKADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 367 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtcatgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 368 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 369 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactatgggtatcggtttggcaatcgccgataaatttgtagaggagggtgcgaaagtagttattactggtcgtcgtgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 370 - protein, 252 aa, synthetic construct
  REGION 1..252: note = Engineered variants of ketoreductase from Lactobacillus kefir
MTDRLKGKVAIVTGGTMGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 371 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactctgggtatcggtttggcaatcgccgataaatttgtagaggagggtgcgaaagtagttattactggtcgtaaggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccactctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 372 - protein, 252 aa, synthetic construct
  REGION 1..252: note = Engineered variants of ketoreductase from Lactobacillus kefir
MTDRLKGKVAIVTGGTLGIGLAIADKFVEEGAKVVITGRKADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTTLGDDLEGAEEMWSQRTKTPMGHIGEPNDIAWICVYLASDESKFATGAEFVVDGGYTAQ

SEQ ID NO: 373 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcatgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccactctgggggatgatctggaaggtgcggaggaattctggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 374 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTTLGDDLEGAEEFWSQRTKTPMGHIGEPNDIAWICVYLASDESKFATGAEFVVDGGYTAQ

SEQ ID NO: 375 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatctggaccactctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 376 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIWTTLGDDLEGAEEMWSQRTKTPMGHIGEPNDIAWICVYLASDESKFATGAEFVVDGGYTAQ

SEQ ID NO: 377 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtcgtatcggtttggcaatcgccgataaatttgtagaggagggtgcgaaagtagttattactggtcgtcgtgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgcggaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 378 - protein, 252 aa, synthetic construct
  REGION 1..252: note = Engineered variants of ketoreductase from Lactobacillus kefir
MTDRLKGKVAIVTGGTRRIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 379 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccgagatgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 380 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTEMGDDLEGAEEMWSQRTKTPMGHIGEPNDIAWICVYLASDESKFATGAEFVVDGGYTAQ

SEQ ID NO: 381 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagacccctctgggggatgatctggaaggtgcggaggaattctggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 382 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEFWSQRTKTPMGHIGEPNDIAWICVYLASDESKFATGAEFVVDGGYTAQ

SEQ ID NO: 383 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtaaggcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccactctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 384 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRKADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTTLGDDLEGAEEMWSQRTKTPMGHIGEPNDIAWICVYLASDESKFATGAEFVVDGGYTAQ

SEQ ID NO: 385 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcgtgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgcggaggaattctggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 386 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEFWSQRTKTPMGHIGEPNDIAWICVYLASDESKFATGAEFVVDGGYTAQ

SEQ ID NO: 387 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcatgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccactctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 388 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPLLGAYNASKGAVRIMSKSAALDCALKDYDVRVNTVHPGAIKTTLGDDLEGAEEMWSQRTKTPMGHIGEPNDIAWICVYLASDESKFATGAEFVVDGGYTAQ

SEQ ID NO: 389 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaatgcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccactctgggggatgatctggaaggtgcggaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 390 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKCVEDTTTEEWRKLLSVNLDGVFFGTRLGIQRMKNKGLGASIINMSSISGLVGDPLLGAYNASKGAVRIMSKSAALDCALKDYDVRVNTVHPGAIKTTLGDDLEGAEEMWSQRTKTPMGHIGEPNDIAWICVYLASDESKFATGAEFVVDGGYTAQ

SEQ ID NO: 391 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcgtgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctggcggacgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 392 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLADDLEGAEEMWSQRTKTPMGHIGEPNDIAWICVYLASDESKFATGAEFVVDGGYTAQ

SEQ ID NO: 393 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgtgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 394 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 395 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcgtgcggatgtaggtgaaaaggccgccaaatcaatcggcggcactgatgttattcgctttgtccagcacgatgcatccgatgaagcaggctggacgaaactgttcgacacgaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctggcttgcgcactgaaggactacgatgtgcgtgtcaacacagtacatccgggcgcgatcaagaccccgctgggggacgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 396 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALACALKDYDVRVNTVHPGAIKTPLGDDLEGAEEMWSQRTKTPMGHIGEPNDIAWICVYLASDESKFATGAEFVVDGGYTAQ

SEQ ID NO: 397 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctggactgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 398 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 399 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatgtgggtcgcaaggccgccaaatcaatcggcggcactgatagcattcgctttgtccagcacgatgcatccgatgaagcaggctggacgaaactgttcgacaccaccgaggaggcattcggcccggttacgaccgtcgtgaacaatgcagggattgcagtttataaaagcgttgaaggtactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 400 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADVGRKAAKSIGGTDSIRFVQHDASDEAGWTKLFDTTEEAFGPVTTVVNNAGIAVYKSVEGTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 401 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 402 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 403 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctgatgtgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 404 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALMCALKDYDVRVNTVHPGAIKTPLGDDLEGAEEMWSQRTKTPMGHIGEPNDIAWICVYLASDESKFATGAEFVVDGGYTAQ

SEQ ID NO: 405 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcgcgcggatgtgggtcgcaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaagctactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 406 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRRADVGRKAAKSIGGTDVIRFVQHDASDEAGWTKLFDTTEEAFGPVTTVVNNAGIIVYKSVEATTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 407 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatcgcggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctgatgtgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 408 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADRGEKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSISGLVGDPTLGAYNASKGAVRIMSKSAALMCALKDYDVRVNTVHPGAIKTPLGDDLEGAEEMWSQRTKTPMGHIGEPNDIAWICVYLASDESKFATGAEFVVDGGYTAQ

SEQ ID NO: 409 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctgatgtgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 410 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALMCALKDYDVRVNTVHPGAIKTPLGDDLEGAEEMWSQRTKTPMGHIGEPNDIAWICVYLASDESKFATGAEFVVDGGYTAQ

SEQ ID NO: 411 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtgggtcgcaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 412 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGRKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 413 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaacgtactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 414 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIIVYKSVERTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 415 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatcgcggtcgcaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctgatgtgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 416 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADRGRKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSISGLVGDPTLGAYNASKGAVRIMSKSAALMCALKDYDVRVNTVHPGAIKTPLGDDLEGAEEMWSQRTKTPMGHIGEPNDIAWICVYLASDESKFATGAEFVVDGGYTAQ

SEQ ID NO: 417 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtgggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaaggtactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctgatgtgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 418 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIIVYKSVEGTTTEEWRKLLSVNLDGVFFGTRLGIQRMKNKGLGASIINMSSISGLVGDPTLGAYNASKGAVRIMSKSAALMCALKDYDVRVNTVHPGAIKTPLGDDLEGAEEMWSQRTKTPMGHIGEPNDIAWICVYLASDESKFATGAEFVVDGGYTAQ

SEQ ID NO: 419 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaaggtactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctggtctgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 420 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEGTTTEEWRKLLSVNLDGVFFGTRLGIQRMKNKGLGASIINMSSIEGLVGDPTLGAYNASKGAVRIMSKSAALVCALKDYDVRVNTVHPGAIKTPLGDDLEGGEEMWSQRTKTPMGHIGEPNDIAWICVYLASDESKFATGAEFVVDGGYTAQ

SEQ ID NO: 421 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatgtgggtgaaaaggccgccaaatcaatcggcggcactgatagcattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 422 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADVGEKAAKSIGGTDSIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 423 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtgggtcgcaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctgtggtgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 424 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGRKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALWCALKDYDVRVNTVHPGAIKTPLGDDLEGGEEMWSQRTKTPMGHIGEPNDIAWICVYLASDESKFATGAEFVVDGGYTAQ

SEQ ID NO: 425 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgtcattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 426 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 427 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatcgcggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 428 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADRGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 429 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctgtggtgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 430 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALWCALKDYDVRVNTVHPGAIKTPLGDDLEGGEEMWSQRTKTPMGHIGEPNDIAWICVYLASDESKFATGAEFVVDGGYTAQ

SEQ ID NO: 431 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 432 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 433 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 434 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 435 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatgtgggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagctactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 436 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADVGEKAAKSIGGTDVIRFVQHDASDEAGWTKLFDTTEEAFGPVTTVVNNAGIAVYKSVEATTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 437 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatgtgggtgaaaaggccgccaaatcaatcggcggcactgatagcattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 438 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADVGEKAAKSIGGTDSIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 439 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 440 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 441 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcgcgcggatcgcggtcgcaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctgatgtgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 442 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRRADRGRKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSISGLVGDPTLGAYNASKGAVRIMSKSAALMCALKDYDVRVNTVHPGAIKTPLGDDLEGGEEMWSQRTKTPMGHIGEPNDIAWICVYLASDESKFATGAEFVVDGGYTAQ

SEQ ID NO: 443 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatcgcggtgaaaaggccgccaaatcaatcggcggcactgatagcattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 444 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADRGEKAAKSIGGTDSIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 445 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctggtctgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 446 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALVCALKDYDVRVNTVHPGAIKTPLGDDLEGAEEMWSQRTKTPMGHIGEPNDIAWICVYLASDESKFATGAEFVVDGGYTAQ

SEQ ID NO: 447 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctgcggtgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 448 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALRCALKDYDVRVNTVHPGAIKTPLGDDLEGGEEMWSQRTKTPMGHIGEPNDIAWICVYLASDESKFATGAEFVVDGGYTAQ

SEQ ID NO: 449 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatcgcggtcacaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 450 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADRGHKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 451 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaacgtactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 452 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIAVYKSVERTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 453 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtgggtcgcaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctgatgtgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 454 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGRKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSIEGLVGDPTLGAYNASKGAVRIMSKSAALMCALKDYDVRVNTVHPGAIKTPLGDDLEGAEEMWSQRTKTPMGHIGEPNDIAWICVYLASDESKFATGAEFVVDGGYTAQ

SEQ ID NO: 455 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatcgcggtcgcaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagatactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctggtctgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 456 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADRGRKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALVCALKDYDVRVNTVHPGAIKTPLGDDLEGAEEMWSQRTKTPMGHIGEPNDIAWICVYLASDESKFATGAEFVVDGGYTAQ

SEQ ID NO: 457 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatagcattcgctttgtccagcacgatgcatccgatgaagcaggctggacgaaactgttcgacaccaccgaggaggcattcggcccggttacgaccgtcgtgaacaatgcagggattattgtttataaaagcgttgaaggtactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 458 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDSIRFVQHDASDEAGWTKLFDTTEEAFGPVTTVVNNAGIIVYKSVEGTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 459 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtgggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 460 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 461 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatgtgggtcgcaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaaggtactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 462 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADVGRKAAKSIGGTDVIRFVQHDASDEAGWTKLFDTTEEAFGPVTTVVNNAGIAVYKSVEGTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 463 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcgcgcggatcgcggtgaaaaggccgccaaatcaatcggcggcactgatagcattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctgatgtgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 464 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRRADRGEKAAKSIGGTDSIRFVQHDASDEAGWTKLFDTTEEAFGPVTTVVNNAGIIVYKSVEDTTTEEWRKLLSVNLDGVFFGTRLGIQRMKNKGLGASIINMSSIEGLVGDPTLGAYNASKGAVRIMSKSAALMCALKDYDVRVNTVHPGAIKTPLGDDLEGGEEMWSQRTKTPMGHIGEPNDIAWICVYLASDESKFATGAEFVVDGGYTAQ

SEQ ID NO: 465 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactcgtggtatcggtttggcaatcgccgataaatttgtagaggagggtgcgaaagtagttattactggtcgtcacgcggatgtaggtgaaaaggccgccaaatcaatcggcggcactgatagcattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 466 - protein, 252 aa, synthetic construct
  REGION 1..252: note = Engineered variants of ketoreductase from Lactobacillus kefir
MTDRLKGKVAIVTGGTRGIGLAIADKFVEEGAKVVITGRHADVGEKAAKSIGGTDSIRFVQHDASDEAGWTKLFDTTEEAFGPVTTVVNNAGIIVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 467 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatgtgggtgaaaaggccgccaaatcaatcggcggcactgatgtcattcgctttgtccagcacgatgcatccgatgaagcaggctggacgaaactgttcgacaccaccgaggaggcattcggcccggttacgaccgtcgtgaacaatgcagggattgcagtttataaaagcgttgaaggtactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagctcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 468 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADVGEKAAKSIGGTDVIRFVQHDASDEAGWTKLFDTTEEAFGPVTTVVNNAGIAVYKSVEGTTTEEWRKLLSVNLDGVFFGTRLGIQRMKNKGLGASIINMSSIEGLVGDPTLGAYNASKGAVRIMSKSSALDCALKDYDVRVNTVHPGAIKTPLGDDLEGAEEMWSQRTKTPMGHIGEPNDIAWICVYLASDESKFATGAEFVVDGGYTAQ

SEQ ID NO: 469 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatcgcggtcgcaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 470 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADRGRKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 471 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatcgcggtcgcaaggccgccaaatcaatcggcggcactgatagcattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 472 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADRGRKAAKSIGGTDSIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 473 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatgtgggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 474 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 475 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatcgcggtgaaaaggccgccaaatcaatcggcggcactgatagcattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 476 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADRGEKAAKSIGGTDSIRFVQHDASDEAGWTKLFDTTEEAFGPVTTVVNNAGIAVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 477 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatagcattcgctttgtccagcacgatgcatccgatgaagcaggctggacgaaactgttcgacaccaccgaggaggcattcggcccggttacgaccgtcgtgaacaatgcagggattattgtttataaaagcgttgaacgtactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 478 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDSIRFVQHDASDEAGWTKLFDTTEEAFGPVTTVVNNAGIIVYKSVERTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 479 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcgcgcggatgtgggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattattgtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattagcgggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtggtgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 480 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRRADVGEKAAKSIGGTDVIRFVQHDASDEAGWTKLFDTTEEAFGPVTTVVNNAGIIVYKSVEDTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGGEEMWSQRTKTPMGHIGEPNDIAWICVYLASDESKFATGAEFVVDGGYTAQ

SEQ ID NO: 481 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgctgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 482 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPLLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 483 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgcagacactaccacggaggaatggcgtaaactgctgtccgttaatctggatggtgtttttttcggcacccgtctgggcattcagcgcatgaaaaataaaggcttgggcgctagcatcatcaatatgagcagtattagtgggctcgtaggcgatccgaccctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 484 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIAVYKSVADTTTEEWRKLLSVNLDGVFFGTRLGIQRMKNKGLGASIINMSSISGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 485 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttaaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaatggtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 486 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIAVYKSVKDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEEWWSQRTKTPMGHIGEPNDIAWICVYLASDESKFATGAEFVVDGGYTAQ

SEQ ID NO: 487 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtatataaaagcgttgaacaaactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 488 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIAVYKSVEQTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 489 - DNA, 759 bp, synthetic construct
  misc_feature 1..759: note = Engineered variants of ketoreductase from Lactobacillus kefir
  source 1..759: mol_type = other DNA
atgaccgatcgtctgaagggcaaagtagccatcgtaaccggcgggactagtggtatcggtttggcaatcgccgataaatttgtagaggagggtgcgaaagtagttattactggtcgtcacgcggatgtaggtgaaaaggccgccaaatcaatcggcggcactgatgttattcgctttgtccagcacgatgcatccgatgaagcaggctggacgaaactgttcgacaccaccgaggaggcattcggcccggttacgaccgtcgtgaacaatgcagggattgcagtttataaaagcgttgaagacactaccacggaggaatggcgtaaactgctgtccgttaatctggatggtgtttttttcggcacccgtctgggcattcagcgcatgaaaaataaaggcttgggcgctagcatcatcaatatgagcagtattgaagggctcgtaggcgatccgacgctgggggcatacaacgcttccaagggggcggtacgtatcatgtcgaaaagcgcagcgctggattgcgcactgaaggactacgatgtgcgtgtcaacacagtacatccgggcgcgatcaagaccccgctgggggatgatctggaaggtgctgaggaaatgtggtcacagcgtacgaaaacccctatgggccacattggcgaaccgaatgacatcgcatggatctgtgtgtacctggcatctgacgaatcgaaatttgcgacgggtgcagaatttgtggtcgacggcgggtataccgcacagtga

SEQ ID NO: 490 - protein, 252 aa, synthetic construct
  REGION 1..252: note = Engineered variants of ketoreductase from Lactobacillus kefir
MTDRLKGKVAIVTGGTSGIGLAIADKFVEEGAKVVITGRHADVGEKAAKSIGGTDVIRFVQHDASDEAGWTKLFDTTEEAFGPVTTVVNNAGIAVYKSVEDTTTEEWRKLLSVNLDGVFFGTRLGIQRMKNKGLGASIINMSSIEGLVGDPTLGAYNASKGAVRIMSKSAALDCALKDYDVRVNTVHPGAIKTPLGDDLEGAEEMWSQRTKTPMGHIGEPNDIAWICVYLASDESKFATGAEFVVDGGYTAQ

SEQ ID NO: 491 - DNA, 759 bp, Lactobacillus kefiri
  source 1..759: mol_type = other DNA
atgactgatcgtttaaaaggcaaagtagcaattgtaactggcggtaccttgggaattggcttggcaatcgctgataagtttgttgaagaaggcgcaaaggttgttattaccggccgtcacgctgatgtaggtgaaaaagctgccaaatcaatcggcggcacagacgttatccgttttgtccaacacgatgcttctgatgaagccggctggactaagttgtttgatacgactgaagaagcatttggcccagttaccacggttgtcaacaatgccggaattgcggtcagcaagagtgttgaagataccacaactgaagaatggcgcaagctgctctcagttaacttggatggtgtcttcttcggtacccgtcttggaatccaacgtatgaagaataaaggactcggagcatcaatcatcaatatgtcatctatcgaaggttttgttggtgatccaactctgggtgcatacaacgcttcaaaaggtgctgtcagaattatgtctaaatcagctgccttggattgcgctttgaaggactacgatgttcgggttaacactgttcatccaggttatatcaagacaccattggttgacgatcttgaaggggcagaagaaatgatgtcacagcggaccaagacaccaatgggtcatatcggtgaacctaacgatatcgcttggatctgtgtttacctggcatctgacgaatctaaatttgccactggtgcagaattcgttgtcgatggtggatacactgctcaataa

SEQ ID NO: 492 - protein, 252 aa, Lactobacillus kefiri
MTDRLKGKVAIVTGGTLGIGLAIADKFVEEGAKVVITGRHADVGEKAAKSIGGTDVIRFVQHDASDEAGWTKLFDTTEEAFGPVTTVVNNAGIAVSKSVEDTTTEEWRKLLSVNLDGVFFGTRLGIQRMKNKGLGASIINMSSIEGFVGDPTLGAYNASKGAVRIMSKSAALDCALKDYDVRVNTVHPGYIKTPLVDDLEGAEEMMSQRTKTPMGHIGEPNDIAWICVYLASDESKFATGAEFVVDGGYTAQ

SEQ ID NO: 493 - protein, 252 aa, Lactobacillus brevis
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSIEGFVGDPSLGAYNASKGAVRIMSKSAALDCALKDYDVRVNTVHPGYIKTPLVDDLPGAEEAMSQRTKTPMGHIGEPNDIAYICVYLASNESKFATGSEFVVDGGYTAQ

SEQ ID NO: 494 - protein, 252 aa, Lactobacillus minor
MTDRLKGKVAIVTGGTLGIGLAIADKFVEEGAKVVITGRHADVGEKAARSIGGTDVIRFVQHDASDETGWTKLFDTTEEAFGPVTTVVNNAGIAVSKSVEDTTTEEWRKLLSVNLDGVFFGTRLGIQRMKNKGLGASIINMSSIEGFVGDPALGAYNASKGAVRIMSKSAALDCALKDYDVRVNTVHPGYIKTPLVDDLEGAEEMMSQRTKTPMGHIGEPNDIAWICVYLASDESKFATGAEFVVDGGYTAQ